(19) United States
(12) Patent Application Publication
(10) Pub. No.: US 2026/0248563 A1
(43) Pub. Date: Aug. 27, 2026

(54) IMAGE-GUIDED NAVIGATION DEVICES, ADAPTERS AND SYSTEMS WITH OPTICAL DISTANCE SENSOR BASED MARKERLESS REGISTRATION, DEPTH DETECTION, IMAGE-GUIDED PREDICTION AND TRAINING MODELS

(71) Applicant: LAMBDA SURGICAL, INC., Wellesley, MA (US)

(72) Inventors: Roman STOLYAROV, Cambridge, MA (US); Bo Yang YU, Winchester, MA (US); Mark STOLYAR, Palmetto, FL (US)

(73) Assignee: LAMBDA SURGICAL, INC., Wellesley, MA (US)

(21) Appl. No.: 19/529,237

(22) Filed: Feb. 4, 2026

Related U.S. Application Data
(60) Provisional application No. 63/755,565, filed on Feb. 7, 2025.
(60) Provisional application No. 63/830,380, filed on Jun. 25, 2025.

(51) Int. Cl. A61B 34/20; A61B 1/00; A61B 1/055; A61B 1/07; A61B 1/317

(52) U.S. Cl. CPC A61B 34/20; A61B 1/00057; A61B 1/00105; A61B 1/055; A61B 1/07; A61B 1/317; A61B 2034/2048; A61B 2034/2055; A61B 2034/2065; A61B 2034/2072

(57) ABSTRACT

A navigation device, adapter or system for real time three-dimensional (3D) depth sensing through endoscope, a laparoscope, an arthroscope, a cystoscope, a ureteroscope, a bronchoscope, a sinuscope, and a borescope devices. The navigation devices or systems can include a modular, releasable adapter that enables LiDAR or other optical time-of-flight sensor with the devices. The devices, adapters or systems are operable to generate sub-millimeter precision reconstructions of the viewed surfaces and can register the surfaces to scans, such as preoperative scans, to facilitate accurate, image-guided procedures, particularly those procedures involving precise actions, such as surgical procedures performed on rigid anatomy. Applications include surgical guidance, augmented reality visualization, distance-based signal normalization, 3D measurement, luminal traversal tracking, and robotic control, all using off-the shelf endoscopes and surgical cameras as well as borescopes. The time-of-flight sensor may be sensitive to a subrange of visible light such that the system readily supports simultaneous acquisition of visible light imaging, fluorescence, and depth data, with calibration workflows for wide field of view optics and adaptive software control of focus, illumination, and display rendering.

### CROSS-REFERENCE

This application claims the benefit of U.S. Provisional Application No. 63/755,565, filed Feb. 7, 2025, and U.S. Provisional Application No. 63/830,380 filed Jun. 25, 2025, which applications are incorporated herein in their entirety by reference as provided for under 37 C.F.R. 1.57(b).

### BACKGROUND

Field: This disclosure relates to image-guided systems with optical distance sensor based markerless registration, such as light detection and ranging (LiDAR) or time-of-flight (TOF) distance sensors. The disclosure also relates to optical distance sensor assisted depth detection. The disclosed technology applies in multiple fields including medical, veterinary and industrial. Calibration and validation of image-guided system, optical distance sensor supervised AI models, and applications of distance sensing spanning digital measurement, intensity quantification, 3D registration, instrument guidance, autonomous robotics, and various procedure specific applications are also disclosed. The functionality can also be integrated into optical distance sensor systems and devices as well as related robotic systems. Adaptive optics, wavelength selectable illumination, thermodynamic compensation and field mapping can also be implemented in the disclosed systems, devices and methods. Prediction and training models are also supported for the image-guided systems.

Background. Accurate real-time environmental visualization is essential for many processes. In the medical device industry accurate real-time visualization during a therapeutic or surgical procedure is important in, for example, endoscopy, arthroscopy, laparoscopy, and luminal procedures. Additionally, accurate real-time visualization is important for boroscopic inspection of machinery and pipelines. However, the lack of spatial information in standard surgical images fundamentally reduces surgical precision, making it difficult to judge the positions and angles of tissues and instruments, assess deformation or spatial constraints, and in general operate using only the two dimensions of information presented in a standard image. This in turn contributes to unnecessary surgical errors, revision procedures, procedure delays, prolonged hospital stays, variable performance across providers, prolonged training times, and rejected cases due to perceived decreased safety. Further, surgeons are often unable to see structures below the tissue surface, even in situations where they must incise, drill, or otherwise operate on the tissue and risk damaging invisible underlying structures. In such cases, surgeons may even opt not to perform a procedure on a patient even though it may be helpful due to increased perceived procedure risk. Additionally, optics can overwhelm scene content with glare, haze, and attenuation if not actively managed. For example, endoscopic platforms used across specialties have visualization constraints when optical pathways operate through liquid columns or spray films, making consistent clarity a cross-domain challenge in humans and animals alike. Additionally, narrower fields of view in procedures, such as endoscopic procedures, as compared to open procedures often lead to surgeons having constrained visualization of the operative field at any given time, further reducing spatial awareness or general awareness of aspects of the scene that are on the periphery of the image being viewed. This reduced awareness further exacerbates the problems described above. Finally, in video-based procedures surgeons often encounter “paradoxical motion” in which the video may move in a different direction than the physical camera, further increasing training time and increasing the likelihood of surgical errors.

### Image-Guided Surgical Systems

Image-guided surgical navigation has been adopted across different surgical specialties to precisely orient surgical instruments relative to anatomical targets. In procedures where high accuracy is required and the target anatomy is sufficiently rigid, the most accurate methods are those which rely on establishing and maintaining a geometric alignment (“3D registration”) between an accurate preoperative scan, typically obtained via CT or MRI, and the physical patient anatomy at the time of the procedure. In such systems, the 3D (three dimensional) registration enables accurate estimation of the positions of anatomical targets to which the surgeon does not have a direct line of sight.

Across medical, veterinary, and industrial domains, there remains a need for dimensionally aware and fluid-aware optics, illumination, and algorithms that explicitly model refraction, absorption spectra, and multiple scattering to stabilize contrast and geometry under changing fluid conditions. Moreover, methods that integrate fluid management with optical design and calibration in-situ are also needed.

Additionally, a primary visual challenge of reviewing a 2D image and translating that image into movement within a 3D environment, such as in endoscopy, is a lack of direct depth perception, which makes it difficult for operators to spatially orient and accurately navigate within complex anatomical structures. A 2D endoscopic image flattens the visual field, forcing the operator to infer spatial relationships and distances from indirect cues like shading, object overlap, and motion parallax, rather than perceiving the spatial relationships directly. An experienced endoscopist often employs adaptive strategies (e.g., moving the camera or instruments deliberately along the z-axis, relying on anatomical knowledge) to compensate for missing depth cues, but these adaptations require extensive practice and may not fully replace true depth perception.

Moreover, the use of 2D imaging can result in measurable errors in sizing, localization, and defect detection that can jeopardize patient safety, prolong procedures, trigger false rejects/escapes. FIGS. 1A-1C provide an exemplar illustration of a target location for a procedure and an actual location achieved by a user showing a preplanned tunnel trajectory vs. an actual distorted trajectory and a corrected trajectory. In FIG. 1A, a surgeon 11 (e.g., user) is performing a procedure on a left knee 13 of a patient using an endoscope assembly 300 with an external 2D display 15. Distortion can arise from camera intrinsic/extrinsic errors, lens distortion, depth bias, and/or registration errors. As will be appreciated by those skilled in the art, navigation use cases extend beyond the illustrated ACL repair to other joints and cavities (hip, shoulder, ankle, small joints, sinus, spine, abdominal and thoracic procedures, and non-medical borescope scenarios) and “tunnel trajectories” can correspond to screw paths, resection planes, debridement zones, and ablation volumes.

More specifically, wide-angle endoscopes and off-axis viewing introduce nonlinear geometric warping that can cause horizontal measurement errors up to 26.4% centrally and up to 65.7% when compounded by angle-of-view effects, which directly affects diagnostic measurements, margin assessment, and tool sizing in clinical workflows. Distortion in endoscopic scenes undermines spatial fidelity for navigation and registration, amplifying placement error risks and forcing conservative intraoperative decision-making and re-localization steps that increase procedure time and cognitive load. In the operating room, geometric distortion degrades consistency between the image and anatomy, complicating instrument targeting, suture spacing, implant alignment, and size estimation, which in turn can prolong cases and increase the likelihood of additional passes or repositioning. In factories, uncorrected distortion reduces algorithmic reliability at the field-of-view periphery and shifts feature positions, degrading pick-and-place accuracy, increasing rework, and propagating error into downstream Statistical Process Control (SPC) metrics and traceability.

Turning to FIGS. 1B-1C, for example, knee anatomy is shown with the femur 12, and the tibia 14 and a surgical tool 21 accessing a ligament 32. FIG. 1B shows a the tip of the distal end of the surgical tool 21 at a target location. Target locations include, for example, where the surgeon 11 intends to drill a tunnel to provide a graft (either from the patient's own body or a donor) to replace the anterior cruciate ligament (ACL). FIG. 1C shows the tip of a distal end of the surgical tool 21 at an actual location that might be achieved based on visualization using currently available endoscopic systems. Targets can also include parts of the anatomy that the surgeon seeks to avoid during the operation, such as a previously placed tunnel when drilling a new tunnel during a revision procedure.

For registration-based navigation systems, the typical workflow can be split into the preoperative phase and the intraoperative phase. The preoperative phase includes at least a scan upload, scan segmentation, and planning. During upload, a previously taken 3D medical scan of the anatomical target area is uploaded to navigation software, including through a standalone system like a USB or CD ROM or from the facility's network, such as a PACS server. To accomplish segmentation, the anatomy of interest is segmented from the scan using a combination of manual and automated methods, where automated methods can include any combination of classical algorithms (such as thresholding, masking, or edge detection) and deep-learning-based approaches. The surgical procedure is planned, with the plan including any combination of instruments (including stylets, needles, scopes, drills, fasteners, implants, etc), associated navigation targets (including entry points target points, trajectories, volumes of interest, resection margins, etc), and ““anti-targets”” or areas to avoid (including critical structures, annotations, safety margins, exclusion areas, etc). Planning can be performed using a combination of manual or automatic methods, where automated methods can include any combination of methods based on first principles physics-based approaches, machine learning such as deep learning, and a priori knowledge of best practices for the given procedure. The planning is performed within a model of the 3D scan and is consequently aligned to the scan's coordinate system.

The intraoperative phase includes at least registration, instrument calibration, and navigation. During conventional registration, the surgeon aligns the preoperative scan (and associated surgical plan) and the physical patient anatomy using marker-based registration (including optical, electromagnetic, or radiographic markers). Marker-based registration typically involves a rigid fixation of an array of fiducial markers to the target anatomy, subsequent determination of the marker position and reference points on the anatomical surface by physically probing the anatomy with a tracked reference probe, and use of this information to determine the coordinate transformation between the markers (which will be used for navigation and detectable throughout the procedure) and the anatomy (which is typically hidden from the line of sight and not directly detectable throughout the procedure). In optical or electromagnetic marker-based methods, the anatomical surface reference points are collected using a probe containing one or more of such markers, whereby the probe is touched to the anatomical surface at a plurality of reference points while the probe's marker positions and those of the rigidly affixed array are simultaneously collected. A priori knowledge of the probe tip location relative to its markers is then used to reconstruct the anatomical surface geometry and position relative to the affixed array, and a 3D alignment between these surface markers and the segmented scan is used to complete the registration. In radiographic marker-based approaches, an intraoperative 3D scan can be taken, the scan detects both the underlying anatomical surface and radio-opaque markers, a segmentation is performed to determine the marker reference points and anatomical surface reference points, and the alignment is subsequently performed.

During instrument calibration, any instruments that will be tracked, whether optically or electromagnetically, may need to be calibrated if the markers on the instrument are in a separate position from the navigated point of interest. Various methods exist to do this and are well described in the existing art. To accomplish navigation, one or more instruments are navigated relative to the anatomical plan using image-based feedback. Feedback can include a combination of graphical representations (such as projections of 3D scan plans with the corresponding instrument position) and quantitative representations (such as the error or distance from the intended target, safety margins, exclusion zones, etc).

Conventional 3D registration is known to be a time-consuming step across different surgical specialties and registration methods. In optical or electromagnetic marker-based registration, the movement of the probe across the anatomical surface can take several minutes to complete at the beginning of the procedure and thus these methods cannot be performed in real-time. This means that unintended obstructions or movements of anatomical targets relative to reference arrays during the procedure might require re-registration. Conventional registration methods can add up to 10 minutes to the case per registration, and even more for re-registration, which may require rolling back certain steps of the procedure, such as draping. Radiographic registration involves the collection of an intraoperative 3D scan, which is not only time consuming but also irradiates the patient in the case of CT. Further, in both of these situations, any accidental misplacement or damage to the fiducial markers that occurs after the registration can require the registration to be repeated in the middle of the procedure, a process which is not only further time consuming but also potentially more cumbersome given the need to interrupt the surgical workflow. Finally, marker-based registration often requires invasive methods, such as the placement of extra incisions to attach the array (as is often performed in spinal surgery) or pin-based frames which puncture the skin (as is often performed in cranial surgery). Finally, because conventional registration takes time to perform, it cannot dynamically adapt to the tissue surface profile changing, as might occur during tissue removal, for example tumor removal or osteoplasty.

Given the cumbersome nature of marker-based 3D registration methods, there has been recent interest in markerless registration. In markerless approaches, a multitude of surface points on the target anatomy is collected using a depth camera or depth estimation approach, and these surface points are used for alignment with the segmented scan. Due to the absence of reference markers for the anatomical target, in existing markerless registration systems the registration can only be maintained throughout the procedure if the target anatomy remains rigid after the initial registration or its surface remains visible to the depth measurement modality throughout the procedure and there is a means to continue measuring the surface geometry and update the registration in real time based on this surface. However, because it involves direct and automatic measurement of the tissue surface, and therefore eliminates the need to manually collect reference points from the tissue surface, such markerless registration approaches have the potential to be less cumbersome and time consuming than marker-based approaches. Further, markerless registration has the potential to reduce trauma for the patient, given that marker placement is often invasive. As an example of such markerless registration approaches, an endoscopic depth measurement system using TOF sensors was described in Roman Stolyarov et al. “Sub-millimeter precision 3D measurement through a standard endoscope with time me of flight,” Proc. SPIE 11949, Advanced Biomedical and Clinical Diagnostic and Surgical Guidance Systems XX, 119490E (7 Mar. 2022).

In summary, markerless registration can be advantageous, but requires the ability to measure the geometry of a surface throughout a process, and especially during navigation. For surgical or boroscopic procedures in particular, this presents a challenge as it means the 3D surface needs to be measured accurately.

FIG. 1D is a diagram of an endoscopic system 40, with an endoscopic probe 41 that includes distal optics 42 and an image relay sensor 43. The endoscopic probe 41 is in communication with a control section 44, a light source and video processor 45, and a display 46. FIG. 1E illustrates a laparoscopic system 50, with a laparoscopic probe 51, digital optics and camera 52, a tubular member 54, handle 57 (user interface), handle controls 55, and display 56. The tubular member 54 can be a plurality of channels including an air channel within its interior, a biopsy/suction channel, and a fluid channel. The air channel (insufflation channel) is a relatively narrow dedicated lumen that is optimized for gas flow and can be used to continuously or intermittently maintain distension. The instrument channel is a larger more robust lumen engineered with withstand mechanical stress and friction from repeated passage of instruments and to tolerate negative pressure during suction. The fluid channel is operable to carry sterile water or irrigation fluid to a nozzle at the tip of the endoscope.

A connection tower 60 is provided connected to the display, insufflator 61, light source 62, and video processor 63. FIG. 1F-G illustrates an arthroscopic system 70 with an irrigation control 71, instrument channel lever 72, suction control 73, distal optics and rod-lens camera 74, an arthroscope handpiece 77 with an insertion tube and instrument channel. A mechanical interface, such as a clamp-type or threaded interface, can be provided to enable the endoscopic system to engage external systems, such as video processors, camera systems, light sources, insufflation systems, fluid systems, and auxiliary control interfaces. The rod-lens camera 74 (or a rod-lens endoscopic imaging system) is a rigid optical system that uses a series of solid glass rods instead of separate small lenses to transmit the image from the distal tip of a rigid endoscope back to a proximal eyepiece or video sensor. Illumination fibers 79 are provided along with an irrigation port 83, and suction lines 84, 84′. An arthroscope tower 80 has a light 75, video processor 81, and pump 82. Information is displayed on a display device 76.

### Industrial Borescope Systems

Industrial borescopes face the same visualization physics: refractive index mismatch, interface reflections, and multiple scattering from turbid coolants or process liquids degrade contrast, depth perception, and metrology accuracy unless illumination geometry, polarization, and fluid-handling are tuned for the medium. As path length and turbidity increase, long-wavelength channels attenuate disproportionately, forcing exposure tradeoffs that can saturate highlights while leaving fine textures under-resolved in shadowed regions. FIG. 1G illustrates components of a borescope 90, including illumination fibers 91, a camera or fiber bundle 92, a flexible insertion tube 93, handle controls 94, a control unit 99 with a light source 95, image processor 97, and display 96. A connector cable 98 connects the borescope to the control unit 99.

To date, none of the current approaches has seen broad adoption in navigation for surgical or boroscopic procedures due to various limitations. Classical visible light imaging approaches such as structure from motion have not been shown to generalize well or be accurate, in part due to the difficulty of estimating camera motion and decoupling it from, for example, tissue motion, and in part due to the lack of features and structure in the scenes. Stereo approaches, while sufficient for providing separate views to either eye for the purpose of 3D perception, have not been accurate enough to achieve intraprocedure registration during surgery or boroscopy. This is largely due to the lack of features and structure and the short stereo camera baseline. Further, in many smaller systems such as 4 mm arthroscopes or sinuscopes, positioning multiple high-resolution cameras in such small cross sections is an unsolved technical challenge. Furthermore, stereo cannot be used to generate a ground truth spatial representation of the surgical scene, and thus stereo approaches are difficult to properly validate. Structured light approaches, while solving for scene featurelessness, suffer from a similar lack of stereo baseline in small diameter systems.

### SUMMARY

Disclosed are devices, adapters, systems and methods for use across medical, veterinary, and industrial domains. Accurate dimensionally aware and fluid-aware optics, illumination, and algorithms that explicitly model refraction, absorption spectra, and multiple scattering to stabilize contrast and geometry under changing fluid conditions. Embodiments disclosed include a navigation system. Suitable navigation systems include, for example, an endoscopic assembly and one or more of each of data processors and data processing systems. Processing circuits or processors are operable to output data derived from one or more of an optical distance sensor and a visible-light imaging sensor to at least one of the display and the navigation system for image-guided procedures. The devices, adapters, systems and methods are operable to compute and display adjusted trajectories that adjusts distortion that can arise from camera intrinsic/extrinsic errors, lens distortion, depth bias, and/or registration errors.

The endoscopic assembly includes an optical TOF camera or optical distance sensor such as a LiDAR camera, and optionally a second imaging device (e.g., video camera or RGB or Red-Green-Blue camera operating at optical wavelengths in the visible light spectrum), both optically coupled to an endoscope having a field of view at a surgical location in a patient's body. The optical distance sensor is operable to generate a real-time depth map, point cloud derived from the depth map in combination with intrinsic camera and lens parameters, or mesh reconstructed from the point cloud of the field of view, where the depth map, point cloud, or mesh can have a resolution finer than 1 mm in all dimensions, and the visible light imaging device is operable to generate a real-time image of the field of view for display to a user of the navigation system. The system further includes a data processing system operable to perform markerless registration (three dimensional spatial correlation) between the point cloud or mesh in real-time to a preoperative scanned image of the surgical location in which the common surface is identified in the scanner image, thereby defining a common three dimensional coordinate system between the depth map and the scanned image. This coordinate system alignment can further be used to provide the user with an overlay on the anatomical surface indicating the locations and nature of underlying structures that are not visible to the naked eye. The coordinate system alignment can further be used to provide real time guidance of surgical instruments wherein the data processing system is used to co-localize the instrument to the common coordinate system. The system further includes a data processing system operable to register the depth map, point cloud, or mesh in real-time to the visible light image.

The endoscopic assembly can be modular so that at least one of the optical distance sensor and the visible light imaging device is releasably coupled to the endoscope. The coupling of the components of the modular assembly can use existing commercial endoscope and camera mounts, and the assembly can include a beam splitter or adaptive optic (such as a rotating shutter) so that both the optical distance sensor and the visible light imaging device can be coupled to the endoscope for simultaneous acquisition of data therethrough. The endoscope is preferably but not limited to be a visible light imaging endoscope having a distal shaft diameter of 12 mm or less and a fiber optic transmission path.

The endoscopic assembly can also integrate rotatability between the RGB camera and the surgical endoscope while locking rotation between the optical distance sensor and the surgical endoscope, given that surgery often requires rotation of the endoscope relative to the RGB camera to visualize more of the surgical scene, while the optical distance sensor may be sensitive to the rotation of the scope, in which the accuracy of the depth map, point cloud, or mesh being a function of the scope rotation.

The endoscopic assembly can also include one or more fiducial markers (fiducial arrays) configured to be tracked wirelessly for identifying the spatial location and orientation of components of the endoscopic assembly. Fiducial arrays include, for example scope fiducial marker array, instrument fiducial marker array, and calibration fiducial marker array. Further, the system can include a surgical tool having one or more fiducial marker (fiducial array) mounted thereto and configured to be wirelessly tracked for identifying the spatial location and orientation of the surgical tool in the common coordinate system. The data processing system can be configured to use tracking data from the fiducial arrays to overlay an image representing at least one of a location and a planned trajectory of the surgical tool onto a visual display of the surgical location, where the display can incorporate any type technology for presenting a real-time image or other representation of the surgical location, preferably including depth data or other three-dimensional information about the surgical location.

In some embodiments, the endoscopic assembly or lens assembly includes an endoscope having a proximal end and a distal end, with an elongated image conduit therebetween, the distal end being positionable at a surgical location within the body of a patient undergoing a surgical procedure. An optical distance sensor camera is optically coupled to the proximal end of the endoscope and configured for acquiring optical TOF data through the image conduit during the surgical procedure. The TOF data can be used to generate a real-time depth map, point cloud, or mesh having a depth resolution finer than 1 mm. A visible light imaging device is also optically coupled to the proximal end of the endoscope for acquiring real-time images through the image conduit during the surgical procedure. A beam splitter or adaptive optic can be included in the endoscopic assembly to enable simultaneous or staggered and aligned acquisition of data through the endoscope by the optical distance sensor camera and the visible light imaging device. The endoscopic assembly can be modular, wherein at least one of the optical distance sensor camera and the visible light imaging device is releasably coupled to the endoscope.

The endoscopic assembly can further include one or more fiducial marker (fiducial array), or a mount for the marker or array, where the marker or array facilitates wirelessly registering the location and orientation of the endoscopic assembly in a three dimensional coordinate system generated from a preoperative scan of the patient's anatomy.

In some aspects, a method for surgical navigation for a surgical procedure that can include the use of the endoscopic assembly and system is provided. The method includes uploading a diagnostic patient scan using any combination of a standalone drive or a wired or wireless connection to a network, segmentation of an anatomical surface of interest from the scan using any combination of manual or automatic methods, and planning of the surgical procedure to include any combination of surgical instruments including but not limited to stylets, needles, endoscopes, drills, fasteners and implants, as well as associated navigation targets (including entry points target points, trajectories, volumes of interest, resection margins, etc), and “anti-targets” or areas to avoid (including critical structures, annotations, safety margins, exclusion areas, etc).

The method further includes assembling the endoscopic assembly and calibrating one or more of acquisition parameters, depth correction coefficients, and intrinsic camera parameters. The method can additionally include mounting fiducial marker arrays on the endoscopic assembly and on a surgical instrument to be used during the surgical procedure. In other calibration steps, the endoscope distal tip position and optical axis orientation are calibrated relative to its marker array using a calibration target, and the instrument tip position and orientation are also calibrated relative to its respective markers.

In surgical procedural steps, endoscopic access to the patient is acquired, followed by visualizing the anatomy of interest and performing automatic and real-time markerless 3D registration of the anatomy with the segmented scan surface using the depth signal obtained from the endoscopic assembly. Then the instrument is navigated relative to the scan and surgical plan using image-based guidance from the display.

The method can include segmentation of the scan or of multiple scans performed on one or more anatomical areas of interest, with any combination of automatic methods including using density or intensity thresholds set by the user and visualized on the display, edge detection, deep learning algorithms, and MRI enhancement algorithms that improve contrast, resolution, or other scan attributes, and manual segmentation or refinement by the user.

Additionally, the planning can include automated planning features like suggestions of surgical instruments, targets, or anti-targets based on optimizing for any combination of tissue geometry, physical properties, location relative to other tissues, detected anatomical landmarks, or any other analyses performed on the scan. The planning can also include automated features relevant to the specific procedure being performed.

In some aspects, a method for obtaining accurate endoscopic depth information in the field of view using the endoscopic assembly is provided. The method includes optimizing acquisition parameters for optical distance sensor imaging by imaging a target presenting a plurality of known depths while sweeping any combination of acquisition parameters including integration time, modulation frequency, binning mode, frame rate, pulse timing, pulse duration, peak power, and shutter timing, and determining which set of parameters produced the best separation and least amount of noise across the imaged depths, then computing a set of depth correction parameters using the optimally determined acquisition parameters by imaging a calibration target presenting a multitude of known depths and using regression to compute a correction function to determine the true depth based on the imaged raw depth and image intensity. Intrinsic optical distance sensor camera calibration parameters are then determined, including principal point, focal length, skew, and distortion parameters by imaging the calibration target and applying a camera calibration algorithm to the images. A point cloud is then computed from the imaged field of view by taking images using the calibration parameters.

Additional embodiments disclosed herein include systems and methods for performing 3D measurement, luminal navigation, and augmented visualization without requiring preoperative registration. The system can generate artificial overlays such as grid patterns or ring projections within lumens, enabling real-time estimation of scope traversal distance, angular position, and position velocity. It further supports integration with robotic surgical systems, including suggestive planning, motion constraints, and autonomous control. Visualization outputs can be presented on stereoscopic display devices or AR/VR headsets, providing the user with a “glass body” perspective aligned to patient anatomy. The system can normalize fluorescence signals by distance and adaptively optimize optical and LiDAR image clarity using autofocus, autogain, and scene-aware illumination. Embodiments may include software-driven calibration using a single target, support for underwater calibration, and compatibility with wide field-of view optics and diverse off-the-shelf camera and endoscope types.

Both the foregoing general description and the following detailed description are exemplary and explanatory only and are not restrictive of the disclosed examples.

### INCORPORATION BY REFERENCE

All publications, patents, and patent applications mentioned in this specification incorporated by reference herein to the same extent as if each individual publication, patent, or patent application was specifically and individually indicated to be incorporated by reference to the maximum extent allowable by law, except for any definitions, disclaimers, disavowals, and inconsistencies.
U.S. Pat. No. 8,504,136B1 issued 2013 Aug. 6 for SEE-THROUGH ABDOMEN DISPLAY FOR MINIMALLY INVASIVE SURGERY;
U.S. Pat. No. 9,119,552B2 issued 2015 Sep. 1 for APPARATUS AND METHOD FOR ENDOSCOPIC 3D DATA COLLECTION;
U.S. Pat. No. 9,123,155B2 issued 2015 Sep. 1 for APPARATUS AND METHOD FOR USING AUGMENTED REALITY VISION SYSTEM IN SURGICAL PROCEDURES;
U.S. Pat. No. 9,262,866B2 issued 2016 Feb. 16 for AUGMENTED REALITY METHODS AND SYSTEMS INCLUDING OPTICAL MERGING OF A PLURALITY OF COMPONENT OPTICAL IMAGES;
U.S. Pat. No. 9,737,239B2 issued 2017 Aug. 22 for SYSTEMS AND METHODS FOR TRACKING BODY SURFACES OF INDIVIDUALS;
U.S. Pat. No. 9,901,409B2 issued 2018 Feb. 27 for SYSTEM AND METHODS FOR INTRAOPERATIVE GUIDANCE FEEDBACK;
U.S. Pat. No. 10,543,045B2 issued 2020 Jan. 28 for SYSTEM AND METHOD FOR PROVIDING A CONTOUR VIDEO WITH A 3D SURFACE IN A MEDICAL NAVIGATION SYSTEM;
U.S. Pat. No. 11,266,465B2 issued 2022 Mar. 8 for QUANTITATIVE THREE-DIMENSIONAL VISUALIZATION OF INSTRUMENTS IN A FIELD OF VIEW;
U.S. Pat. No. 11,417,491B2 issued 2022 Aug. 16 for EMITTER, ELECTRON GUN IN WHICH SAME IS USED, ELECTRONIC DEVICE IN WHICH SAME IS USED, AND METHOD FOR MANUFACTURING SAME;
U.S. Pat. No. 11,464,578B2 issued 2022 Oct. 11 for SYSTEMS, METHODS, APPARATUSES, AND COMPUTER-READABLE MEDIA FOR IMAGE MANAGEMENT IN IMAGE-GUIDED MEDICAL PROCEDURES;
U.S. Pat. No. 11,547,499B2 issued 2023 Jan. 10 for DYNAMIC AND INTERACTIVE NAVIGATION IN A SURGICAL ENVIRONMENT;
U.S. Pat. No. 11,563,911B2 issued 2023 Jan. 24 for METHOD AND SYSTEM FOR TIME-OF-FLIGHT IMAGING WITH HIGH LATERAL RESOLUTION;
U.S. Pat. No. 11,617,492B2 issued 2023 Apr. 4 for MEDICAL THREE-DIMENSIONAL (3D) SCANNING AND MAPPING SYSTEM;
U.S. Pat. No. 11,798,183B2 issued 2023 Oct. 24 for MACHINE LEARNING TECHNIQUES FOR PREDICTING DEPTH INFORMATION IN IMAGE DATA;
U.S. Pat. No. 11,918,310B1 issued 2024 Mar. 5 for INTERACTIVE EXTENDED-REALITY SYSTEM FOR ROBOTIC MEDICINE;
U.S. Pat. No. 11,928,834B2 issued 2024 Mar. 12 for SYSTEMS AND METHODS FOR GENERATING THREE-DIMENSIONAL MEASUREMENTS USING ENDOSCOPIC VIDEO DATA; U.S. Pat. No. 11,935,239B2 issued 2024 Mar. 19 for CONTROL METHOD, APPARATUS AND PROGRAM FOR SYSTEM FOR DETERMINING LESION OBTAINED VIA REAL-TIME IMAGE;
U.S. Pat. No. 11,977,218B2 issued 2024 May 7 for SYSTEMS AND METHODS FOR MEDICAL IMAGING;
U.S. Pat. No. 11,998,281B1 issued 2024 Jun. 4 for EXTENDED-REALITY OPERATING ROOM FOR ROBOTIC SURGERY;
U.S. Pat. No. 12,063,338B2 issued 2024 Aug. 13 for AUGMENTED REALITY GUIDANCE FOR SPINAL SURGERY WITH STEREOSCOPIC DISPLAYS AND MAGNIFIED VIEWS;
U.S. Pat. No. 12,086,998B2 issued 2024 Sep. 10 for AUGMENTED REALITY GUIDANCE FOR SURGICAL PROCEDURES;
U.S. Pat. No. 12,125,223B2 issued 2024 Oct. 22 for TIME-OF-FLIGHT DEPTH ENHANCEMENT;
U.S. Pat. No. 12,127,795B2 issued 2024 Oct. 29 for AUGMENTED REALITY DISPLAY FOR SPINAL ROD SHAPING AND PLACEMENT;
U.S. Pat. No. 12,131,818B2 issued 2024 Oct. 29 for SYSTEMS AND METHODS FOR PROVIDING SURGICAL GUIDANCE;
U.S. Pat. No. 12,154,239B2 issued 2024 Nov. 26 for LIVE SURGICAL AID FOR BRAIN TUMOR RESECTION USING AUGMENTED REALITY AND DEEP LEARNING;
U.S. Pat. No. 12,161,428B1 issued 2024 Dec. 10 for SYSTEM FOR COMPUTATION OF OBJECT COORDINATES ACCOUNTING FOR MOVEMENT OF A SURGICAL SITE FOR SPINAL AND OTHER PROCEDURES INCLUDING INTERPOLATION OF VERTEBRAL POSITION AND ORIENTATION;
US20170181809A1 published 2017 Jun. 29 for ALIGNMENT OF Q3D MODELS WITH 3D IMAGES;
US20180042466A1 published 2018 Feb. 15 for COMPACT ENDOSCOPE DESIGN FOR THREE-DIMENSIONAL SURGICAL GUIDANCE;
US20210196098A1 published 2021 Jul. 1 for SURGICAL SYSTEM CONTROL BASED ON MULTIPLE SENSED PARAMETERS;
US20210326694A1 published 2021 Oct. 21 for DISTANCE DETERMINATIONS USING ONE OR MORE NEURAL NETWORKS;
US20220012954A1 published 2022 Jan. 13 for GENERATION OF SYNTHETIC THREE-DIMENSIONAL IMAGING FROM PARTIAL DEPTH MAPS;
US20220050206A1 published 2022 Feb. 17 for SYSTEM AND METHOD FOR IMPROVING 3D SENSOR MEASUREMENT ACCURACY;
US20220079675A1 published 2022 Mar. 17 for AUGMENTED REALITY GUIDANCE FOR SURGICAL PROCEDURES WITH ADJUSTMENT OF SCALE, CONVERGENCE AND FOCAL PLANE OR FOCAL POINT OF VIRTUAL DATA;
US20220125519A1 published 2022 Apr. 28 for AUGMENTED REALITY ASSISTED JOINT ARTHROPLASTY;
US20220240786A1 published 2022 Aug. 4 for SYSTEM AND DEVICES FOR MULTISPECTRAL 3D IMAGING AND DIAGNOSTICS OF TISSUES, AND METHODS THEREOF;
US20220383530A1 published 2022 Dec. 1 for METHOD AND SYSTEM FOR GENERATING A DEPTH MAP;
US20230000331A1 published 2023 Jan. 5 for ENDOSCOPE SYSTEM, ENDOSCOPE, AND DISTANCE CALCULATION METHOD;
US20230181258A1 published 2023 Jun. 15 for ROBOTIC SURGICAL SYSTEM FOR INSERTION OF SURGICAL IMPLANTS;
US20230181263A1 published 2023 Jun. 15 for DYNAMIC 3D SCANNING ROBOTIC LAPAROSCOPE;
US20230190136A1 published 2023 Jun. 22 for SYSTEMS AND METHODS FOR COMPUTER-ASSISTED SHAPE MEASUREMENTS IN VIDEO;
US20230245333A1 published 2023 Aug. 3 for APPARATUS AND A METHOD FOR ESTIMATING DEPTH OF A SCENE;
US20230267679A1 published 2023 Aug. 24 for METHOD AND SYSTEM FOR RECONSTRUCTING THE THREE-DIMENSIONAL SURFACE OF TUBULAR ORGANS;
US20230273357A1 published 2023 Aug. 31 for DEVICE AND METHOD FOR IMAGE PROCESSING;
US20230277249A1 published 2023 Sep. 7 for APPARATUS, SYSTEMS, AND METHODS FOR INTRAOPERATIVE INSTRUMENT TRACKING AND INFORMATION VISUALIZATION;
US20230316639A1 published 2023 Oct. 5 for SYSTEMS AND METHODS FOR ENHANCING MEDICAL IMAGES;
US20230352133A1 published 2023 Nov. 2 for SYSTEMS AND METHODS FOR PROCESSING MEDICAL DATA;
US20230363831A1 published 2023 Nov. 16 for MARKERLESS NAVIGATION SYSTEM;
US20230380659A1 published 2023 Nov. 30 for MEDICAL THREE-DIMENSIONAL (3D) SCANNING AND MAPPING SYSTEM;
US20230393278A1 published 2023 Dec. 7 for ELECTRONIC DEVICE, METHOD AND COMPUTER PROGRAM;
US20240111048A1 published 2024 Apr. 4 for COAXIAL TIME-OF-FLIGHT OPTICAL FIBER DISTANCE MEASUREMENT;
US20240155241A1 published 2024 May 9 for MEDICAL OBSERVATION SYSTEM, INFORMATION PROCESSING DEVICE, AND INFORMATION PROCESSING METHOD;
US20240225732A9 published 2024 Jul. 11 for COAXIAL TIME-OF-FLIGHT OPTICAL FIBER DISTANCE MEASUREMENT USING DUAL COMB RANGING;
US20240237898A1 published 2024 Jul. 18 for ENDOSCOPY BASED ON OPTICAL COHERENCE TOMOGRAPHY (OCT) AND CONVOLUTIONAL NEURAL NETWORKS (CNNS);
US20240249408A1 published 2024 Jul. 25 for SYSTEMS AND METHODS FOR TIME OF FLIGHT IMAGING;
US20240303838A1 published 2024 Sep. 12 for ABSOLUTE DEPTH ESTIMATION FROM A SINGLE IMAGE USING ONLINE DEPTH SCALE TRANSFER;
US20240320935A1 published 2024 Sep. 26 for SYSTEMS, METHODS AND DEVICES FOR AUGMENTED REALITY ASSISTED SURGERY;
US20240324848A1 published 2024 Oct. 3 for INFORMATION PROCESSING APPARATUS, INFORMATION PROCESSING METHOD, AND INFORMATION PROCESSING PROGRAM;
US20240324849A1 published 2024 Oct. 3 for INFORMATION PROCESSING APPARATUS, INFORMATION PROCESSING METHOD, AND INFORMATION PROCESSING PROGRAM;
US20240324852A1 published 2024 Oct. 3 for SYSTEMS AND INTERFACES FOR COMPUTER-BASED INTERNAL BODY STRUCTURE ASSESSMENT;
US20240331176A1 published 2024 Oct. 3 for METHOD AND ELECTRONIC DEVICE FOR DETERMINING DEPTH INFORMATION OF OBJECTS IN A SCENE;
US20240331182A1 published 2024 Oct. 3 for SYSTEMS AND METHODS FOR GENERATING THREE-DIMENSIONAL MEASUREMENTS USING ENDOSCOPIC VIDEO DATA;
US20240353669A1 published 2024 Oct. 24 for SYSTEMS AND METHODS FOR MEDICAL IMAGING;
US20240386686A1 published 2024 Nov. 21 for ELECTRONIC DEVICE INTERLOCKED WITH ENDOSCOPE TO PERFORM ENDOSCOPIC EXAMINATION AND METHOD FOR PERFORMING ENDOSCOPIC EXAMINATION USING THE SAME;
US20240420356A1 published 2024 Dec. 19 for METHOD AND SYSTEM FOR DEPTH ESTIMATION USING GATED STEREO IMAGING; and
Stolyarov et al., “Sub-millimeter precision 3D measurement through a standard endoscope with time of flight,” Proc. SPIE 11949, Advanced Biomedical and Clinical Diagnostic and Surgical Guidance Systems XX, 119490E (7 Mar. 2022).

### BRIEF DESCRIPTION OF THE DRAWINGS

The novel features of the teachings are set forth with particularity in the present disclosure. As will be appreciated by those skilled in the art, the various features illustrated in the drawings may not be drawn to scale. Moreover, the illustrations presented in the present disclosure are not meant to be actual views of any particular apparatus (e.g., device, system, etc.) or method, but are merely representations that are employed to describe various examples of the disclosure. Accordingly, the dimensions of the various features may be arbitrarily expanded or reduced in the figures for clarity. In addition, some of the drawings may be simplified for clarity. Thus, the drawings may not depict all of the components of a given apparatus (e.g., device) or all operations of a particular method. The novel features of the disclosure are set forth with particularity in any claim presented.

A better understanding of the features and advantages of the present disclosure will be obtained by reference to the following detailed description that sets forth illustrative examples, in which the principles of the disclosed teachings are utilized, and the accompanying drawings of which:

FIGS. 1A-1C provide an exemplar illustration of a 2D imaging system and target anatomical location for a device engagement during a procedure and an actual anatomical location achieved by the device based on currently available technology; FIG. 1D is a functional representation of an endoscopic system; FIG. 1E is a functional representation of a laparoscopic system; FIG. 1F is a functional representation of an arthroscopic system; FIG. 1G is a functional representation of a borescope;

FIG. 2 is a diagram showing a system incorporating LiDAR;

FIG. 3A is a diagram of an exemplary endoscopic assembly according to an embodiment, including a releasable multichromatic camera and coupler with integrated light source; FIGS. 3B-1 and 3B-2 are lateral views of an endoscope with a coupler FIG. 3C illustrates a system including the endoscopic assembly of FIG. 3A in use with a screen interface showing multiple images simultaneously;

FIG. 4 is a diagram of an exemplary endoscopic assembly according to an embodiment, including an integrated multichromatic camera and an optical distance sensor adapter with releasable scope coupling and integrated light source;

FIG. 5 is a diagram of an exemplary endoscopic assembly according to an embodiment, including a releasable multichromatic camera and endoscope coupling, and an external light source;

FIG. 6 is a perspective view of a calibration fixture according to an embodiment;

FIG. 7 is a schematic representation of a calibration fixture immersed in a saline solution;

FIG. 8 schematically illustrates an exemplary navigation system according to an embodiment providing visual guidance for a surgeon performing an arthroscopic surgical procedure;

FIG. 9 is an exemplary process flow diagram for surgical navigation according to an embodiment;

FIG. 10 is a schematic diagram of a navigation system according to an embodiment providing an alternative visualization perspective to a surgeon utilizing an augmented reality/virtual reality headset;

FIGS. 11A-D are schematic screen shot of a user interface of the navigation system;

FIG. 12 is a screen shot of a user interface that shows contour lines as an overlay and traversal distance for a scope inserted into a lumen;

FIG. 13 is a schematic representation of a computer system for use with embodiments disclosed herein;

FIGS. 14A-14C are screen shots of a process of using a user interface of a depth measurement system to obtain dimensional information;

FIG. 15 is a screenshot of a visual representation of 3D information generated by a depth measurement system separate from an image captured by the depth measurement system; and

FIG. 16 is a chart illustrating different sterile arrangements for exemplary endoscopic assemblies.

### DETAILED DESCRIPTION

Throughout the disclosure and figures the following markings have been used: a axis; proximal 10 or proximal position; and distal or distal 20 position. The proximal position reflects the location nearest the user's hand in use; the distal position reflects the location furthest from the user's hand in use. The axis is an axis about which an action occurs.

In order to understand the operational aspects of the systems, methods and devices disclosed herein, it is helpful to understand there are three anatomical planes generally used in anatomy to describe the human body and structure within the human body: the axial plane, the sagittal plane and the coronal plane. The sagittal plane is an imaginary vertical plane that divides a body into right and left sections. The axial plane is an imaginary horizontal plane that divides a body into upper (superior) and lower (inferior) parts. The coronal plane is an imaginary vertical plane (perpendicular to the sagittal plane) that divides a body into front (anterior) and back (posterior) sections.

The systems are operable to normalize fluorescence signals by distance and adaptively optimize optical and optical distance sensor image clarity using autofocus, autogain, and scene-aware illumination. optical distance sensor, for example, provides a remote optical distance sensing device and method that uses light in the form of a pulsed laser to measure distances and create high-resolution 3D models of objects or the environment. optical distance sensor functions by emitting laser pulses and measuring the time it takes for the light to be reflected back to a sensor, a technique also known as “time-of-flight” (TOF).

The systems can also include software that provides software-driven calibration using a single target, support for underwater calibration, and compatibility with wide field-of view optics and diverse off-the-shelf camera and optical imaging devices and systems. The cameras can include, for example, a camera port to provide a connection for data transfer.

### I. System Overview

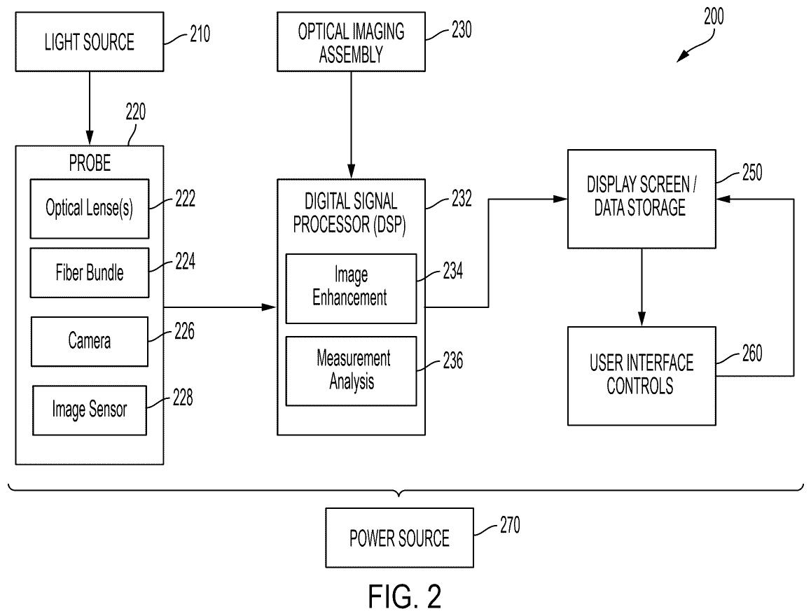

FIG. 2 is a diagram showing an optical imaging system 200 incorporating an optical distance sensor such as LiDAR (e.g., “light detection and ranging” or “laser imaging, detection, and ranging”). As will be appreciated by those skilled in the art, LiDAR provides visible light imaging depth imaging modality based on transmitting illumination and measuring an amount of time the transmitted illumination takes to return to a camera. optical distance sensor systems are configurable to measure tissue geometry to sub-millimeter accuracy. An illumination interface can be provided that is configured to deliver wavelength-specific distance-sensing illumination and broadband visible-wavelength illumination into the common light path of the lens assembly, wherein the illumination interface is configured to be mechanically coupled and subsequently uncoupled from the common light path. The housing can also comprise a mechanical coupler operable to releasably attach to a visible light camera.

Moreover, optical distance sensor components are operable to work with a conventional scope due to its visible light imaging nature and may be couplable, coupled and uncoupled as needed. Further, because optical distance sensor can be used to obtain depth information and/or distance data, optical distance sensor can also be used to train deep-learning-based models toward providing depth and distance measurements in the absence of the optical distance sensor module. Distance data and image data can be generated for overlapping portions of the field of view and then analyzed, processed and/or manipulated by the system.

The optical imaging system 200 is a complete system that uses a lens assembly to focus light onto a target (e.g., sensor or film), turning optical data into a usable image or video, and capture one or more images. The lens assembly can be a grouping of multiple lenses, mirrors, and/or filters in mounts, operable to control delivery of light for a specific optical task. Light can be delivered via, for example, an integrated fiber-optic common light path. The lens assembly can have a lens assembly distal end and a lens assembly proximal end, with either end being couplable to another component of the system.

The optical imaging system 200 can be formed integrally with an optical imaging device and comprise an optical distance sensor coupled to an optical imaging device comprising a probe and a display screen. The optical distance sensor can be part of a removable adapter. The optical imaging system 200 can comprise several subcomponents including a light source 210, a probe 220, an optical imaging assembly 230 (such as a LiDAR assembly), a display screen 250 and data storage system, user interface controls 260, and a power source 270. The data storage can include raw depth frames, rectified depth maps, registration parameters, trajectories, and intraoperative measurements. Any suitable export format can be used and later retrieved for machine learning model training, quality assurance, or outcome analysis.

An optical imaging device can include an eyepiece port or dedicated connection point. The connection point can be part of a trinocular head, designed to attach cameras or other imaging devices, that allows capture and view of magnified images digitally instead of just through traditional eyepieces, sometimes sharing the optical path with the eyepieces via a beam splitter or switch. The beam splitter is operable to take one incoming beam and produce two outputs (e.g., a transmitted and a reflected beam) with some specified relationship in power, polarization, or wavelength. In reverse, the beam splitter is operable as a beam combiner (e.g., beam combiner) that can be used as a beam combiner, taking two appropriately arranged input beams and overlapping them into one output beam. An illumination interface can be provided that is configured to couple a distance-sensing illumination source at a first wavelength band and visible-wavelength illumination at a second wavelength band into the common light path of the lens assembly through a beam combiner.

The probe 220 further comprises an optical lens 222, a fiber bundle 224, a camera 226, and an image sensor 228. The digital signal processor 232 can further comprise an image enhancement component 234 and a measurement analysis component 236. The display screen/data storage 250 can be integrally formed as part of the overall system or wirelessly connected. User interface controls 260 can likewise be integrally formed as part of the overall system or wirelessly connected. A processor can be configured to output data derived from one or more of the optical distance sensor and the visible-light imaging sensor to at least one of the display and the navigation system for image-guided procedures.

The light source 210 is in communication with the probe 220 and the probe 220 is in communication with the optical imaging assembly 230. Fiducial markers can be provided that are disposed on a housing and configured to be detected by an external tracking system to determine a spatial position.

Processing unit: A processing unit can take input from the optical imaging assembly 230, camera 226, and an instrument tracker, and provide output to a graphic user interface and a display screen/data storage 250 that supports a user's ability to perform a navigated procedure. Software can be provided that enables the user to upload and segment pre-procedure scan(s) or information, create and refine procedure plans within the uploaded scan(s), perform any necessary calibration of the optical imaging assembly 230 (such as a LiDAR assembly) or instrument tracking subsystems, and receive real-time image-guided navigation during the appropriate phases of the procedure. The processing unit can include processing circuits or processors that are operable to output data derived from one or more of the optical distance sensor and the visible-light imaging sensor to at least one of the display and the navigation system for image-guided procedures.

Display screen/data storage 250 (e.g., touch screen display): The display screen can be used to interface with the software to facilitate performing all of steps in a workflow, seeing an output of segmentation, planning, or navigation, and providing user input to any other functions of the software.

The light source 210 is configurable to provide wavelength selectable illumination that dynamically adjusts specific wavelengths (colors) of light administered to a target by the light source. In some configurations the light source is configurable to allow a user to select or change a wavelength for illumination. The light source 210 can also emit a fixed, broad spectrum of light (for example, white light) or a single, fixed wavelength (like a standard laser). The use of a selectable or adjustable wavelength leverages the fact that different materials absorb, reflect, or transmit light differently depending on the wavelength, thus allowing for enhanced control, contrast, and analysis in various scientific and industrial applications. In some configurations, the optical imaging system 200 allows a user to identify the target surface for illumination which then allows the optical imaging system 200 to select an optimal wavelength based on the identified target surface material or properties. In some configurations, tunable Lasers are provided. Tunable lasers include light sources (such as dye lasers, free-electron lasers, or semiconductor lasers) where the output wavelength can be continuously adjusted by changing the physical properties of the laser medium or by using external wavelength-selective elements like gratings or filters. One or more filters (e.g., interference filters, shortpass/longpass filters, or filter wheels) can also be used with a broadband light source to allow a user to select a narrow band of wavelengths to pass through. In some configurations, monochromators are provided that use diffraction gratings and slits to disperse light from a broadband source and physically select a desired narrow band of wavelengths to be output. Machine vision and optical communications, technologies like Acousto-Optic Tunable Filters (AOTFs) or Liquid Crystal on Silicon (LCoS) based switches can electronically and rapidly select and route specific wavelengths of light without mechanical movement.

The optical imaging devices can be in communication with a navigation tower that manages multiple devices or adapters to provide centralized processing, shared collaboration, and remote updates.

### A. Robotic Integration

Robotic integration of the optical imaging sensor functionality may be implemented by mounting the imaging sensor assembly on an articulated robotic manipulator, and operatively coupling its field of view to the robot's programmable motion so that images are captured from predetermined poses along a multi-axis trajectory relative to the target article or environment. In certain embodiments, the robotic controller registers the kinematic coordinate frame of the robot with the optical sensor coordinate frame, enabling calibrated position- and orientation-dependent image acquisition that supports functions such as inspection, alignment, localization, or guidance. The robotic arm can thereby position the optical imaging sensor with repeatable sub-millimeter accuracy at multiple working distances and viewing angles, optionally in coordination with additional sensors (for example, force or depth sensors) to implement closed-loop adjustments based on image-derived metrics or detected features. Furthermore, as many robots have integrated vision systems, robotic integration can also be performed using AI trained on a dataset collected using the TOF sensor in which the TOF data is collected in such a way that it shares a field of view with the RGB data, is digitally aligned to the RGB data, and is synchronized with the RGB data. If such a dataset is collected, a machine learning algorithm could be using to infer TOF information using RGB video alone and result in creation of a software model that can infer such information. If the content of the dataset used to train the model is sufficiently similar to the scenes encountered in normal operating conditions for the robot, and the model is cross validated to the required accuracy level for the procedure, this software model can then be deployed as part of a robotic system without the need for additional hardware.

### B. Thermodynamic Compensation

Thermodynamic compensation can also be incorporated into the disclosed optical systems, devices, adapters and methods to improve performance-critical parameters, such as focal length, alignment, or sensor calibration, by ensuring such parameters remain substantially invariant over a specified range of temperatures despite thermally induced changes in material and structural properties. In representative implementations, compensation is achieved by combining materials with offsetting thermo-optic coefficients and thermal expansion characteristics, by configuring lens groups or mechanical linkages so that temperature-driven dimensional changes counteract refractive index variations, or by applying active corrections based on measured temperature or thermally induced wavefront errors. This approach mitigates effects such as thermal lensing, focus drift, and thermally driven aberrations, thereby maintaining optical throughput and image quality without requiring extended warm-up periods or frequent manual recalibration under varying thermal loads. Compensation can also be applied to thermal effects on TOF illumination characteristics including phase delay, modulation frequency, peak power, rise time, fall time, and others. This can be done either in open loop or using temperature measurements obtained from the TOF laser or laser driver circuit during option. Compensation methods can include a combination of mathematical techniques to adjust the sensor data based on the laser temperature, or operational gating based on the laser temperature.

### C. Wavelength Selectable Illumination

The disclosed optical systems, devices, adapters and methods can also incorporate wavelength selectable illumination and associated illumination architecture in which the spectral content of the emitted light is dynamically controlled so that one or more discrete wavelength bands can be selected, switched, or tuned on demand for a given imaging or sensing operation. In representative implementations, the optical systems, devices, adapters and methods can include a broadband or multiwavelength source (for example, an array of LEDs or a supercontinuum source) in combination with wavelength-selective elements such as interference filters, acousto-optic or liquid-crystal tunable filters, monochromators, or electronically controlled laser modules, enabling software-configurable selection of illumination wavelength, bandwidth, and intensity. This capability allows tailoring of contrast mechanisms, penetration depth, and signal-to-background ratio for different targets or modalities—such as fluorescence channels, absorption features, or scattering signatures—while using a common optical path and detector.

### D. Prediction and Training Models

Prediction and training models can also be employed to provide to data-driven computational frameworks that are configured to learn quantitative relationships between measured input variables and desired output metrics. The models can then use the learned mappings to infer or forecast system behavior for new data. These models can include, for example, regression algorithms or neural networks that are trained on labeled datasets comprising sensor measurements, environmental or operating parameters, and associated ground-truth outputs such as performance indicators, alignment errors, or image-quality scores. During a training phase, model parameters are iteratively adjusted to minimize a loss function that penalizes discrepancies between predicted and known outputs, after which the trained model is deployed in a prediction mode to generate real-time or near-real-time estimates that can support tasks such as calibration, anomaly detection, performance optimization, or closed-loop control of an optical or imaging system.

### E. Calibration and Validation

Further disclosed embodiments can use or incorporate calibration and validation processes to ensure that a measurement system or predictive model produces accurate and trustworthy results. The calibration process determines and adjusts a relationship between system outputs and known reference standards or ground-truth data so that measured or simulated quantities faithfully represent physical reality under specified conditions. The validation process assesses the performance of the calibrated system or model against independent data not used during calibration, with the goal of establishing confidence that the resulting measurements or predictions remain accurate and reliable when applied to operational scenarios or new conditions.

The optical imaging systems, devices, adapters and methods can also include field mapping to determine and represent how points or regions in an object or scene map to corresponding locations within the optical distance sensor's field of view or across the illuminated or detected optical field. In representative implementations, a calibration procedure is used to characterize this spatial mapping by relating object-space coordinates to detector pixel coordinates, often via a lookup table or transformation function that accounts for distortion, magnification, and other system imperfections. The resulting field map enables accurate reconstruction, registration, and quantitative analysis of image data, including correction of spatial nonuniformities or misalignments across the field.

### F. Procedure Specific Metrics

Outcomes can be analyzed and compared with platform or procedure specific predicted outcomes. Procedure-specific outcomes in the medical context, for example, can include a mix of diagnostic yield/efficacy, symptom/QoL improvement, and complication or retreatment rates for each procedure. For example, during an ACL procedure one or more of structural/clinical status; graft failure or re-tear, side-to-side laxity (e.g., KT-1000), pivot-shift grade, need for revision or additional meniscal procedures may be evaluated. Additionally complications for infection, stiffness/arthrofibrosis and hardware-related problems may be considered.

### II. Medical Devices

In some aspects, the systems, devices, adapters and methods enable real-time three-dimensional (3D) sensing that may be readily integrated with various medical procedure devices (e.g., optical imaging devices and systems), including low-cost endoscopic systems, such as single channel endoscopes, and flexible endoscopes. The 3D sensing may be implemented using a time of flight sensor that is sensitive to a subrange of the visible spectrum. Elements may be combined into a module that is structured and/or calibrated to provide sub-millimeter resolution. Such a depth sensing system may also support robotic motion constraints (e.g., go/no-go zones), suggestive or autonomous movement, or visual overlays aligned with tracked instruments enabling a broader class of surgical robotics and navigation capabilities.

The resolution of an optical imaging device depth measuring module may be enhanced via a calibration process, including a calibration fixture that may hold, for example, an endoscope tip in a deterministic position with respect to a multidimensional target. The fixture may include a container, with the target in the container. The container may be filled with saline or other medium representative of the environment in which the optical imaging device will be used. Accurate calibration of the depth measurement system relative to the optical imaging device tip may be facilitated with such a calibration fixture. When the module includes or couples an image sensor or other type of sensor to the optical imaging device in addition to a TOF sensor, these additional sensors may be calibrated also to the TOF sensor. The time-of-flight sensor is configurable to determine a distance based on a time delay or phase shift between emitted distance-sensing light and returned distance-sensing light.

Disclosed adapters can be configured to be releasable from an underlying optical imaging device, such as an endoscope. Disclosed adapters can also be configured to be releasable from a camera. The camera can be a visible light camera positionable within the imaging device or a camera module as a whole (comprising optics plus image sensor plus housing/interface) that acquires images, such as visible-spectrum images. A variety of twist or clamp devices can be used to enable the adapters to securely engage the optical imaging device and/or camera. Illumination can be transmitted through a light guide, such as a light guide made of a transparent medium, such as glass, plastic or optical fiber that transports light from a first location to a second location. The light guide can be a common light guide. Suitable illumination includes, for example LiDAR illumination (e.g., actively generated laser light used by a LiDAR system). Separate light sources can be provided or integrated. Moreover rotation immobilization can be achieved relative to an optical imaging device but not with respect to a multichromatic camera. For example, the housing can comprise a mechanical interface operable to immobilize the optical distance sensor relative to the lens assembly while permitting movement of the visible-light imaging sensor relative to the lens assembly.

As will be appreciated by those skilled in the art, adapters can also be hermetically designed for sterilization when required. The focus features may also be electronically adjusted or mechanically adjusted. Adapters may also provide, for example, visible light LiDAR and/or infrared LiDAR. A variety of power sources can be provided to power the adapter including cable, power cord, and battery.

Embodiments disclosed herein may provide optical imaging device capability for measuring distances or diameters within lumens, overlaying grid markers, or visualizing path traversal through tubular anatomy. Further, techniques as described herein may be combined in a system that can normalize fluorescence signals (e.g., ICG) based on working distance or provide real time visualizations to a surgeon, such as “glass body” overlays aligned to internal structures. As another example, a module as described herein may operate on diverse off-the-shelf endoscopes, compensate for wide field-of-view distortion, or operate with flexible plug-and-play calibration under fluid conditions.

Turning to the figures, specific non-limiting embodiments are described in further detail. It should be understood that the various systems, components, adapters, features, and methods described relative to these embodiments may be used either individually and/or in any desired combination as the disclosure is not limited to only the specific embodiments described herein.

### A. Endoscope

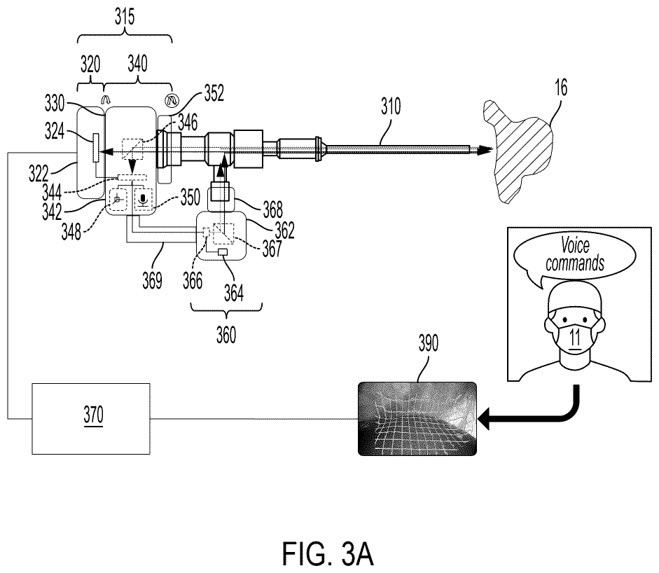

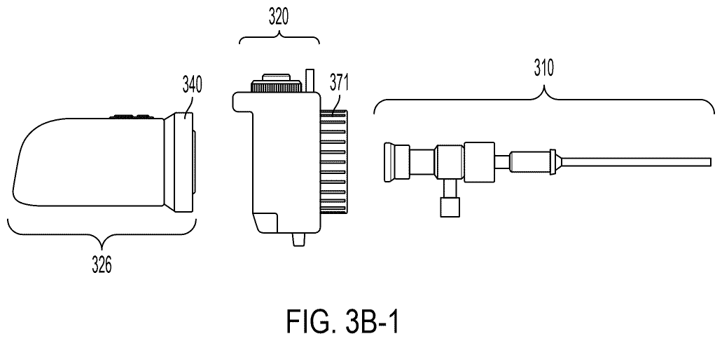

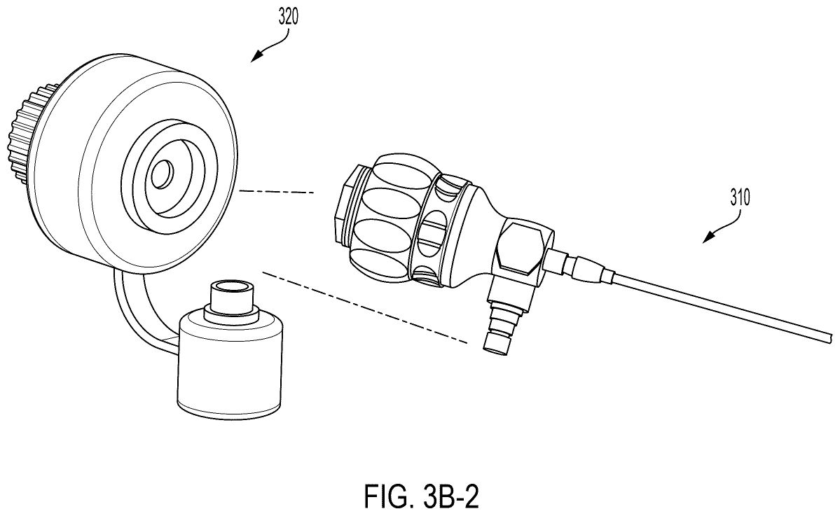

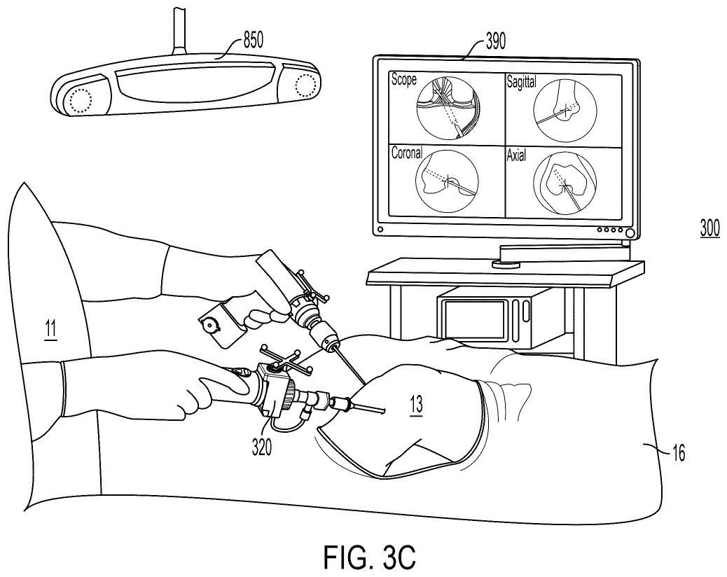

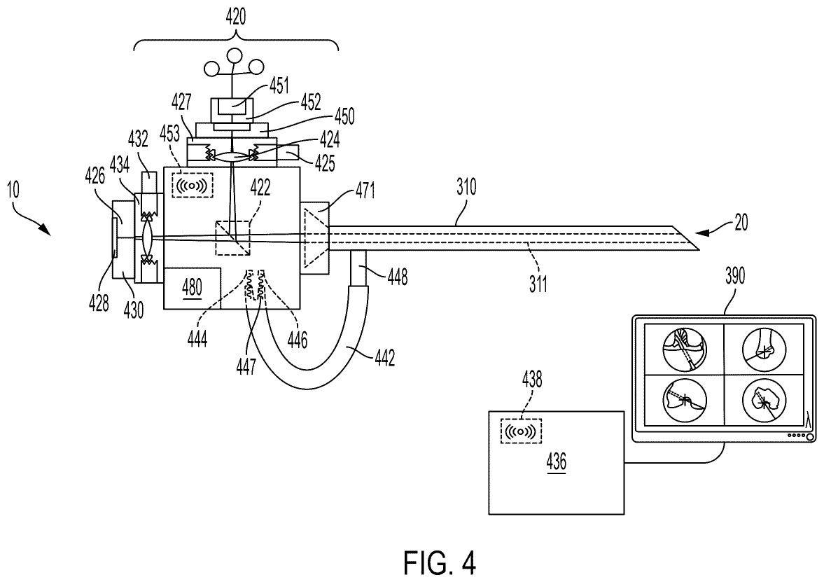

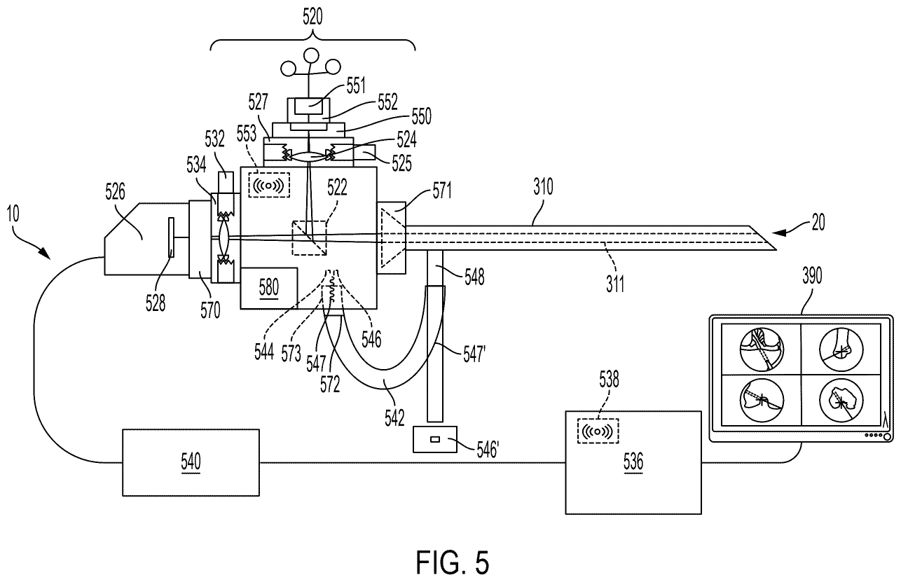

FIG. 3A through FIG. 5 schematically illustrate an exemplary optical imaging device assembly, such as an endoscope assembly 310, as an example implementation of a depth sensing module 320 or depth sensing sensor assembly, as part of an optical imaging device or system. Other medical device optical imaging device assemblies that can incorporate the disclosed technology or use a disclosed adapter, include, but are not limited to: laparoscopes, arthroscopes, endoscopes, gastroscopes, colonoscopes, cystoscopes, uteroscopes, hysteroscopes, bronchoscopes, rhinoscopes, thoracoscopes, colposcopes and amnioscopes. A gentrainierally cylindrical adapter housing is sleeved over and coaxial with the scope shaft so that its centerline coincides with the optical axis of the lens assembly to define an axis of rotation. The rotational coupling between the adapter housing and the scope body (illustrated in FIGS. 4 and 5 as the interface between the adapter block carrying the optical distance sensor optics and the fixed scope barrel), which allows the adapter housing (and thus the lens/imaging components) to rotate 360 degrees about the longitudinal axis.

FIG. 3A schematically illustrates use of an exemplary navigation system according to an embodiment for performing a minimally invasive endoscopic procedure at a surgical location in a patient's body. An endoscopic assembly and a surgical instrument such as a surgical drill or any other surgical instrument, with one or more fiducial marker arrays rigidly attached thereto for simultaneous respective tracking of position and orientation of the instrument in three-dimensional space using an optical instrument tracker having stereo cameras in this construction.

The illustrated system has an endoscope 310 in communication with a sensor assembly 315 or sensor module. The sensor assembly 315 is in communication with processor 370 and display device 390 and processor 370 is in communication with the display device 390. The distal endoscope tip 312 engages a target anatomical surface 16, and the surgeon 11 operates the system while viewing and engaging the display device 390. The display device can be engaged by any suitable mechanism including, touch, input device (e.g., mouse, keyboard, pen), voice (microphone and/or speech-recognition module that is local or cloud-based), gestures, eye gaze, haptic (e.g., wearables), and multimodal combinations thereof. Suitable commands include, for example, freeze depth, record measurement, save plane, switch view to coronal, increase gain, etc. An illuminator assembly 360 is also provided that engages the sensor assembly at a first end and the endoscope at a second end.

The sensor assembly 315 can further comprise a multichromic light sub-assembly 320 and an optical distance sub-assembly 340. The multichromic light sub-assembly 320 further includes a multichromic light sub-assembly housing 322 with a multichromic light sensor 324 positioned thereof. The optical distance sub-assembly 340 can have a separate optical distance sub-assembly housing 342. The optical distance sub-assembly housing 342 can house a beam routing element 346, an optical distance sensor 344, an IMU device 348, and a microphone 350. The beam routing element can be a beam splitter or adapting optic, such as a rotating shutter. The IMU device can be any suitable inertial measurement unit device or sensor, including, but not limited to MEMS, fiber optics, accelerometers, gyroscopes, magnetometers, and devices that measure linear and/or angular motion and orientation providing movement information about the device. Movement information includes orientation information which can be used to correct the horizon of an image thereby enabling an orientation of a displayed image to correspond to a gravity orientation. Additionally, movement information can include position information usable to digitally combine views from multiple camera angles. Combined views can then be used as a simulated stereo camera to further refine distance information. Combined views can also be used to stitch spatial information across an image scene to create a larger spatial representation of an environment than is visible through the field of view lens assembly at any given time. As will be appreciated by those skilled in the art, one or more components can be housed separately provided suitable communication is provided between components. Moreover, a single housing can be used in some configurations without departing from the scope of the disclosure.

The multichromic light sub-assembly 320 and optical distance sub-assembly 340 can be configured to have a rotatable connection between the multichromic light sub-assembly housing 322 and the optical distance sub-assembly housing 342. A suitable rotatable connection is achieved by, for example, providing a rotating bearing 330 that matingly engages the two housings, i.e., the multichromic light sub-assembly housing 322 and the optical distance sub-assembly housing 342 that are in contact. The opposing side of the optical distance sub-assembly housing 342 can have a releasable coupler 352 which is operable to rotatably engage the endoscope 310. Thus the overall sensor assembly 315 is rotatably engaged at the endoscope end.

The illuminator assembly 360 engages the sensor assembly 315 at one end and the endoscope 310 at another end. As illustrated, the illuminator assembly 360 engages the optical distance sub-assembly 340. The illuminator assembly 360 has an illuminator assembly housing 362 housing a multichromic light source 364, and beam source 366 for generating a wavelength for detection by the optical distance sensor 344. The beam source 366 can be any suitable device, such as a laser, that generates a beam in a desired wavelength. The illuminator assembly 360 can also house a beam combining element 367, or wavelength combining element, operable to combine the outputs from the multichromic light source 364 and the beam source 366 before delivering the combined output to the endoscope 310. A thread scope coupler 368 can be provided for engaging the endoscope 310. Input from the sensor assembly 315 can be received by the illuminator assembly 360 via a conduit 369 positioned therebetween. The conduit 369 can further include a carrier to prevent rotation between the sensor assembly 315 and the endoscope 310.

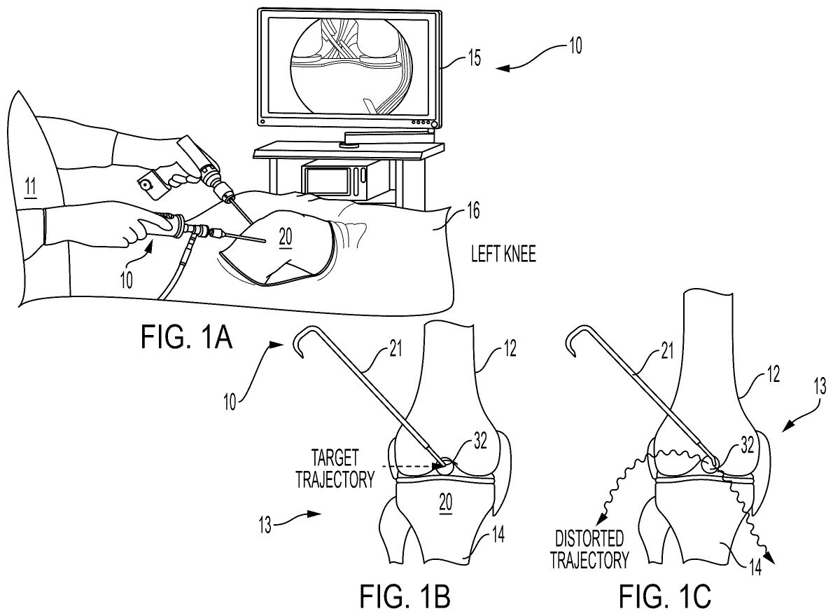

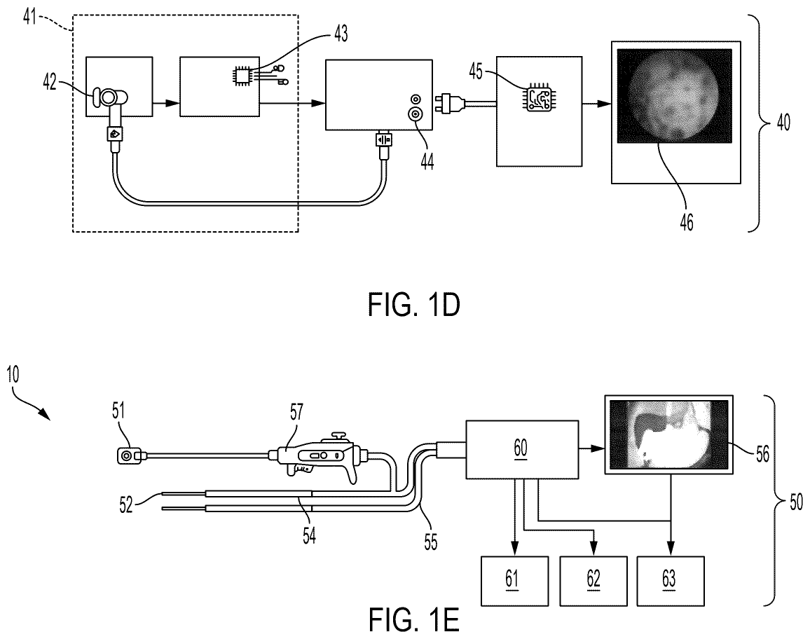

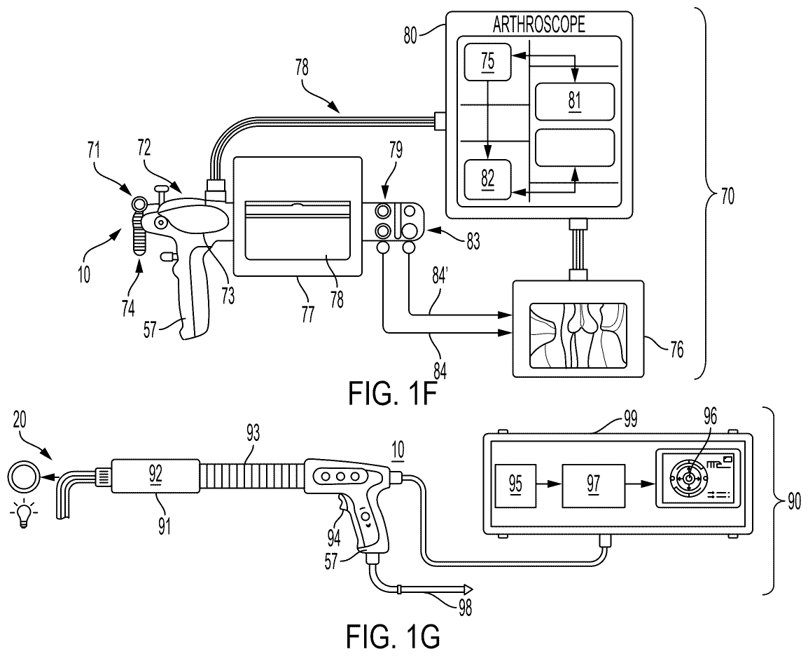

FIG. 3B-1 is a side view of an exemplar assembly with an endoscope 310, a depth sensing module 320 in the form of an adapter that engages an endoscope 310 on a first surface and engages a multichromatic camera body 340 of a multichromatic camera 326 on another surface. The multichromatic camera can be a broadband multichromatic camera. FIG. 3B-2 another view of an exemplar assembly with an endoscope 310, a depth sensing module 320 adapter.

Systems according to some embodiments have an optical distance sensor camera 324 (or any other suitable time of flight camera) releasably mounted (FIG. 3A) to or integrated with (FIG. 4) an endoscope, a multichromatic camera (or any other suitable visible light imaging device), and at least one light guide in any combination. Such systems preferably include one or more mechanical elements to prevent rotation of the endoscope, light guide, or multichromatic camera relative to the optical distance sensor camera 324 or illumination source. Such an arrangement may be desirable to ensure lens distortions as seen by the sensor remain constant throughout operation. In some embodiments, such systems may be sterilizable.

FIG. 3C illustrates an endoscope assembly 300 with an instrument tracker 850, a display device 390, and a depth sensing module 320 incorporated into the endoscope. The surgeon is shown holding the endoscopic assembly in one hand and a drill in the other hand, in a position in which they are ready to perform a navigated task, such as placing a tunnel into the bone using the drill. The endoscopic assembly and drill are uniquely distinguishable, both of which can be optically tracked by an instrument tracker 850. The endoscopic assembly obtains depth information from the surface where the procedure will be performed (such as the intracondylar notch of the femur) and relays this depth information to the processing unit; which computes a 3D reconstruction of the surface using the supplied depth information and previously obtained depth and camera calibration parameters, and then performs a registration of this 3D reconstructed surface to the segmented scan surface (for example, the intracondylar notch surface previously segmented by the user from the diagnostic MRI scan) to localize the scan to a common coordinate system. Simultaneously, the processing unit obtains input regarding fiducial marker array positions from the instrument tracker 850, and combines this with the marker-to-tip transformations previously computed during instrument calibration to determine the position of the navigated instrument (in this case the drill bit or K wire being used to place the tunnel) in the common coordinate system. Then, once both the MRI scan and the instrument are localized to this common coordinate system, the processing unit computes projected 2D views showing the instrument position relative to the MRI in different planes, which can include the anatomical cardinal planes (axial, coronal, sagittal) or other planes such as those references to the instrument position (for example, in-line and perpendicular) and renders these views onto the screen, along with the visible light image obtained by the multichromatic camera. The visible light image could also include quantitative metrics such as angular or position errors of the instrument relative to the anatomical trajectory. In this case, the surgeon then uses this image-based navigation to guide the placement of the tunnel.

The navigation system further comprises a data processing unit configured to acquire signals from the instrument tracker, the optical distance sensor camera, and the multichromatic camera, process them into an image-based navigation signal, and display the signal on a display device 390 as one or more visible images and/or data. In an embodiment, the signal displayed on the display device 390 can display three dimensional information about the surgical location, or a three-dimensional image. In another embodiment, the signal displayed on the display device 390 includes a plurality of different perspective views of the surgical location such as Scope, Sagittal, Axial and Coronal in FIG. 3C. In yet another embodiment the signal displayed on the display device 390 includes surgical tool position and trajectory information relative to a surgical landmark identified using position and orientation information acquired from depth data generated using depth data from the endoscopic assembly.

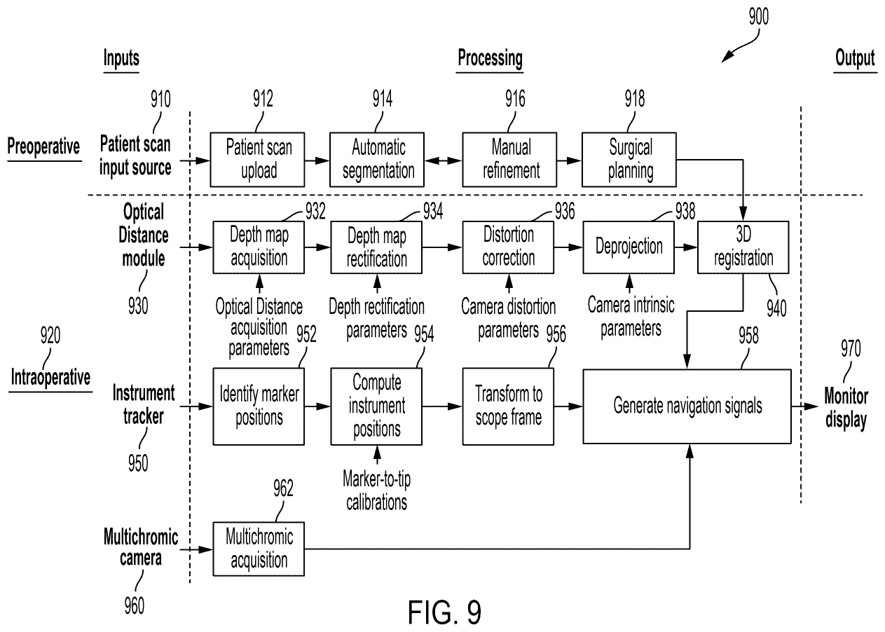

In some embodiments, the data processing unit additionally provides a guided workflow that allows a user to upload a patient scan, segment the scan, create a surgical plan, perform intraoperative 3D registration, calibrate the locations of points of interest on the optical distance sensor module and instruments relative to that instrument's respective marker array, and perform the procedure while obtaining real-time guidance from the display device. One such workflow is described in relation to FIG. 9 below.

The intended use of navigation systems according to some embodiments is to provide real-time intraoperative guidance for surgeons performing endoscopic procedures requiring precise positioning of instruments relative to anatomical structures. Areas of applicability include but are not limited to drilling or fixation in specific anatomical locations. Examples include navigating the position and trajectory of tunnel placement (for example, in arthroscopic ligament reconstructions), navigating the position or trajectory of anchors or fasteners (for example, anchor placement in arthroscopic rotator cuff repair or pedicle screw placement in minimally invasive spinal fusion), navigating the biopsy or excision of a solid tumor or lesion (for example, laparoscopic excision of kidney tumors or endoscopic excision of sinus tumors), navigating the removal of bone during arthroscopic osteotomy, and navigating the biopsy or excision of endotracheal or endoesophageal tumors or lesions.

Navigation information provided to the user may comprise the estimated position of one or more surgical instruments within a map of the target area provided by a preoperative scan (usually CT or MRI). The map can also contain or incorporate one or more surgical plans of the target area specified by the user prior to performing the procedure, and the navigation information can include a display of the one or more surgical plans and the navigation instrument's position relative to the surgical plan(s).

In some embodiments, the optical imaging device navigation system is compatible with standard optical imaging device equipment and comprises an optical distance sensor-based depth estimation module (with associated calibration equipment), an instrument tracker, a disposable set of fiducial markers, a computational processing unit, and a touchscreen display device.

In nonlimiting embodiments, the optical imaging navigation system comprises standard optical imaging device, such as endoscope equipment, including an endoscope, camera system, standard surgical instruments and disposables, a standard diagnostic scan: CT or MRI of the target anatomy taken prior to a surgical procedure, and further including optical distance sensor based depth estimation devices that are in some embodiments sterilizable, wireless, battery-powered, and comprise an imaging adapter that sits in between a standard endoscopic multichromatic camera and an endoscope, releasably attaching to each using their respective coupling mechanisms or coupling hardware (for example, a C-mount thread to the camera and a universal adapter to the scope). The coupling hardware can operate as an illumination interface.

FIGS. 4-5 illustrates an exemplar embodiment of the depth sensing module 420 where the multichromatic sensor 428 is an integrated sensor. No optical distance sensor acquisition unit is provided. Additionally, because the multichromatic sensor 428 is integrated, there is no optical distance sensor camera housing engaged via a second housing coupler 471.

The depth sensing module may include integrated light sources (FIG. 3A and FIG. 4) or a remote, external light illumination source (FIG. 5) (e.g., an off the-shelf light source) or optical distance sensor illumination sources. In some cases, the depth sensing module is sterilizable. Using a light source for at least the optical distance sensor camera integrated into a module with the optical distance sensor camera may be desirable to maintain accuracy of the readings obtained by the optical distance sensor camera. Specifically, in order to achieve a sufficiently high-resolution depth map (e.g. less than a millimeter resolution), the optical distance sensor illumination source may be electronically synchronized with respect to the optical distance sensor camera. In some embodiments, the optical distance sensor illumination source may be a pulsed illumination sources or a continuous wave illumination source. Additionally, the lengths of the optical paths for the light source and camera to and from a common point may be known and constant, allowing for such a synchronization. In some cases, at least the optical distance sensor illumination source may be rigidly connected to the optical distance sensor camera, and guided to the tip of the scope by a flexible light guide. In some embodiments, the optical distance sensor illumination source is optically coupled to the endoscope via direct abutment of the optical distance sensor illumination source to the light post of the endoscope. Such an arrangement may reduce signal variability caused by movement of such a flexible light guide.

An embodiment of a depth sensing module 420 includes a beam splitter assembly 422 that takes received light from the endoscope's lens and splits off light within a predetermined wavelength range (such as infrared light) to an optical distance sensor camera 424 through an adjustable focus knob, and relays the rest of the light to the endoscope camera for standard visible light imaging video visualization through a separate adjustable focus knob. The depth sensing module 420 includes the optical distance sensor illumination source, which itself contains a mechanical interface to a surgical light guide which can be used to couple light from the illumination source to the endoscope's illumination path which in an embodiment comprises optical fiber. In an embodiment, when commanded, the TOF system transmits light at timed intervals through the fiber optic system and into the tissue space, receives the reflected light at the sensor through the scope's rod-lens system, and uses the spatially resolved time of flight information to construct a real-time pixel-wise depth map. The timing and duration transmitted pulses and camera shutter integrations are computed in an acquisition parameters optimization process completed during system installation or servicing.

Optical distance sensor camera 424 calibration target: The optical distance sensor camera 424 calibration target is a device used for camera calibration (determination of intrinsics including focal point, principal point and skew, as well as distortion parameters) of the optical distance sensor system when it is assembled with the endoscope. It includes one or more recognizable calibration patterns such as checkerboards, charuco boards, april tags, dot grids, or other types of fiducial patterns that can be seen and recognized by the optical distance sensor system.

The optical distance sensor depth calibration target: The optical distance sensor depth calibration target is a device used for depth rectification of the optical distance sensor system when it is assembled with the scope. This ensures that all depth measurements are referenced to an origin located at the center of the scope tip, and along axes aligned to the scope's imaging plane and axis. This calibration would in effect remove edge-based depth distortion and ensure that a Cartesian transformation can be found between the optical distance sensor system markers and the computed depth map.

The endoscope assembly 310 illustrated includes an endoscope with an endoscope rod-lens receiving path in communication with a depth sensing module 420 that includes a beam splitter assembly 422 for optically coupling the endoscope 310 to both the optical distance sensor camera 424 (e.g., a LiDAR camera having a LiDAR camera lens), and a multichromatic camera, such as an RGB camera positioned in a multichromatic camera body or housing. The multichromatic camera, e, g, visible-light imaging camera, can include a multichromatic sensor 428, a multichromatic camera lens 430, a multichromatic camera focus knob 432, and a multichromatic adjustable focus assembly 434. A multichromatic wavelength source can be provided that generates one or more wavelengths in a visible spectrum. Typical between 380 nm to 750 nm. Wavelengths can also be generated more specifically within the red (610 nm-630 nm)-green (530 nm-555 nm)-blue (460 nm-470 nm) spectrums. A multichromatic wavelength sensor can be provided that senses one or more wavelengths within the 380 nm to 750 nm wavelength range. An optical distance wavelength source can be provided that generates one or more wavelengths in the infrared range, e.g., from 700 nm to 1 mm, more commonly 905 nm (near-infrared) and 1550 nm (short-wave infrared). An optical distance wavelength sensor can be provided that senses one or more wavelengths within the 700 nm to 1 mm wavelength range.

A processing unit 436 can be provided with a wireless processing unit communication module 438 (optional). The processing unit 436 can be in communication with the multichromatic camera via a visible light imaging acquisition unit 441.

In some configurations, the assembly does not require couplers, e.g., where LiDAR functionality is incorporated into the optical imaging device or system.

The depth sensing module 420 can be configured to include respective light sources for the optical distance sensor camera 424 and for the multichromatic camera, e.g., LiDAR illumination source 444 and light source 446. An internal light guide bifurcation 447 can be provided in the light guide 442. In some embodiments the light sources provide illumination through the endoscope via a light guide 442 coupled to, for example, a light post of the endoscope. One or more illumination sources may be provided that are coupled through direct abutment to an endoscopic light post 448 or other light input to a transmit path in the endoscope 410. The beam splitter assembly 422 is operable to direct received incident optical distance sensor illumination (non-visible light) to the optical distance sensor camera 424 and to direct incident light illumination (e.g., visible light) to the multichromatic camera. However, it is contemplated that in some configurations the optical distance sensor illumination may be visible light having a first predetermined wavelength range (e.g. a wavelength range corresponding to blue light, to red light, etc.). The beam splitter assembly 422, may be a wavelength selective beam splitter configured to direct incident visible light having the first predetermined wavelength range to the optical distance sensor camera 424 while directing incident visible light at a second predetermined wavelength range to the multichromatic camera 426 (which can include video recording capabilities). The second predetermined wavelength range may include all wavelengths of visible light except for the wavelengths in the first predetermined wavelength range.

The beam splitter assembly 422 may be any beam splitter assembly 422 that is suitable. The beam splitter assembly 422 may include a dichroic beam splitter arranged to relay the first predetermined wavelength range to the optical distance sensor camera 424 and the second predetermined wavelength range to the multichromatic camera.

The beam splitter assembly 422 may also comprise a beam splitter operable to split all wavelengths of the light from the optical imaging device, such as the endoscope, to direct all wavelengths towards both an optical distance sensor 450 and the multichromatic camera 426. For instance, the beam splitter assembly 422 may direct 50% (or any other suitable percent) of the intensity of the light from an optical imaging device, such as an endoscope, toward the multichromatic camera, and 50% (or any other suitable percent) of the intensity of the light from the endoscope toward the optical distance sensor camera 424. A bandpass filter can be provided that is configured to preferentially pass light in the first predetermined wavelength range. The bandpass filter may be disposed between the optical distance sensor 450 of the optical distance sensor camera 424 and the beam splitter assembly 422 in order to block light outside of the first predetermined wavelength range from reaching the optical distance sensor 450. A notched filter configured to preferentially pass light in the second predetermined wavelength range and be disposed between the sensor of the multichromatic camera 426 and the beam splitter assembly 422 to filter the incident light relayed toward the optical distance sensor 450.

In various embodiments, the optical distance sensor camera 424 and the multichromatic camera 426 have respective optical elements (e.g. camera lenses and focus controls) located in an optical path between the first housing coupler 470 and the sensors for each of the respective cameras as part of an optical imaging system. Using an endoscope 310 as an exemplar optical imaging device, as illustrated in FIGS. 3A-5 the endoscope includes an endoscope rod-lens receiving path 311, but any type of endoscope capable of transmitting optical distance sensor and visible image data to the respective cameras can be used in endoscopic assemblies of some embodiments (e.g. a flexible fiber-optic endoscope). The endoscopic assembly can also include wireless or wired connections to a data processing unit (a processing unit having at least one processor), and in some embodiments the endoscopic assembly includes a battery to function as a power supply that can include providing power for wireless connections with the processing unit.

The endoscopic assembly can further comprises one or more fiducial marker (a fiducial marker array 451) or a fiducial marker array mount 452 configured for attaching a fiducial marker array 451 to a depth sensing module housing, the position of which can be optically tracked to enable registration of the spatial position and orientation of the endoscopic assembly to a coordinate system defined by a preoperative scan of the patient.

Systems according to some embodiments have an optical distance sensor camera (or any other suitable TOF camera) releasably mounted to or integrated with an endoscope, a multichromatic camera (or any other suitable visible light imaging device), and at least one light guide in any combination. Such systems preferably include one or more mechanical elements to prevent rotation of the endoscope, light guide, or multichromatic camera relative to the optical distance sensor camera or illumination source. Such an arrangement may be desirable to ensure lens distortions as seen by the sensor remain constant throughout operation. In some embodiments, such systems may be sterilizable.

An optical distance sensor camera 424 can be integrated as part of a depth sensing module having a housing containing a beam splitter assembly 422. The optical distance sensor camera 424, has an optical distance sensor camera focus knob 425, and an adjustable focus assembly 427. The optical distance sensor camera 424 can be positioned in an optical distance sensor camera housing that is in communication with an optical distance sensor 450, a fiducial marker array 451 via a fiducial marker array mount 452 and an optical distance sensor wireless processing unit communication module 453.

The depth sensing module 420 can be contained in a depth sensing module housing which has a first housing coupler 470, a second housing coupler 471, and a third housing coupler 472 when the depth sensing module 420 is configured to adapt an existing optical imaging device to provide optical distance sensing functionality. The depth sensing module is configured to be in communication with a distance-sensing illumination source. The depth sensing module can be coupled to the distance-sensing illumination source.

The first housing coupler 470 can be operable to mechanically connect the depth sensing module 420 to an endoscope 410, the second housing coupler 471 can also be operable to mechanically connect the depth sensing module 420 to a multichromatic camera 426, the third housing coupler 472 can be operable to mechanically connect the depth sensing module 420 to a while light source 446. Each of the first housing coupler, second housing coupler, and third housing coupler can be formed integrally with a depth sensing module housing and operable to mechanically engage a mechanical interface on the endoscope 310, multichromatic camera 426, and a while light source 446.

A suitable power source can be provided, including a removable battery 480. Output from the depth sensing module 420 can be displayed on suitable display device 390 such as a touch screen display.

In some embodiments, the depth sensing module may also include removable battery 480 for powering the various components of the depth sensing module 420. At least one fiducial marker array 451 is mounted to the optical distance sensor module. The module housing may be releasably mountable to an endoscope, a multichromatic camera, and/or a light guide. In some embodiments, the depth sensing module includes a first coupler configured to couple the endoscope to the module housing, a second coupler configured to couple the multichromatic camera to the module housing, and/or a third coupler configured to couple the light guide to the module hosing. In some embodiments the first coupling, the second coupling, and/or the third coupling include one or more mechanical elements to prevent rotation of the endoscope, light guide, or multichromatic camera relative to the optical distance sensor camera 424 and/or an illumination source (which may be integrated into the module housing in some embodiments, as shown in FIGS. 3A-6). The depth sensing module may include adjustable focus knobs for the visible light imaging and optical distance sensor camera 424 in some cases.

In some embodiments, the depth sensing module may include an optical distance sensor wireless processing unit communication module to allow communication between the depth sensing module and a remote processing unit including at least one processor (e.g. to allow for transmission of optical distance sensor camera 424 data). In some embodiments, the communication module, e.g., the processing unit wireless communication module or the optical distance sensor wireless processing unit communication module, is a wired communication module. The processor(s) may be configured to receive the optical distance sensor camera 424 data and to compute a point cloud based on the data.

The system can further includes a visible light imaging acquisition unit electrically connected to the multichromatic camera body and also connected to a processing unit 436 containing a wireless processing unit communication module 438 to exchange information with a corresponding wireless processing unit communication module 438 in the depth sensing module 420. A display device 390 (e.g., monitor or touch screen monitor) displays four images transmitted from the processing unit 436 as described in more detail below.

A beam splitter assembly 422 relays received light within a first predetermined wavelength range to the optical distance sensor camera 424 and relays light within a second predetermined wavelength range to the multichromatic camera. In some embodiments, the first predetermined wavelength range corresponds a subset of white (visible) light (e.g. blue light, red light, etc.), and the second predetermined wavelength range corresponds to white light, or corresponds to white light other than the first predetermined wavelength range. In some embodiments, the first predetermined wavelength range correspond to infrared or near infrared light.

Adjustable focus knobs, e.g. optical distance sensor camera focus knob 425 and multichromatic camera focus knob 432, may be included in some embodiments which are operable to allow adjustment of focal length for both the visible light imaging and optical distance sensor camera 424. An optical distance sensor wireless processing unit communication module 453 may be included in some cases which transmits the depth information from the optical distance sensor camera 424 to a processing unit 436. The processing unit 436 may receive information from the off-the-shelf visible light imaging acquisition unit and depth sensing module, perform the processing including 3D reconstruction, registration, and instrument tracking, and display the navigation signal onto a connected screen, and/or perform any other suitable function.

The endoscope assembly 310 illustrated includes an endoscope with an endoscope rod-lens receiving path in communication with a depth sensing module that includes a beam splitter assembly 422 for optically coupling the endoscope 310 to both the optical distance sensor camera 424 (e.g., a LiDAR camera having a LiDAR camera lens), and a multichromatic camera, such as an RGB camera positioned in a multichromatic camera body or housing. The multichromatic camera, e,g, visible-light imaging camera, can include a multichromatic sensor 428, a multichromatic camera lens 430, a multichromatic camera focus knob 432, and a multichromatic adjustable focus assembly 434.

A processing unit 436 can be provided with a wireless processing unit communication module 438 (optional). The processing unit 436 can be in communication with the multichromatic camera via a visible light imaging acquisition unit 441.

In some configurations, the assembly does not require couplers, e.g., where LiDAR functionality is incorporated into the optical imaging device or system.

The depth sensing module 420 can be configured to include respective light sources for the optical distance sensor camera 424 and for the multichromatic camera, e.g., LiDAR illumination source 444 and light source 446. An internal light guide bifurcation 447 can be provided in the light guide 442. In some embodiments the light sources provide illumination through the endoscope via a light guide 442 coupled to, for example, a light post of the endoscope. One or more illumination sources may be provided that are coupled through direct abutment to an endoscopic light post 448 or other light input to a transmit path in the endoscope 410. The beam splitter assembly 422 is operable to direct received incident optical distance sensor illumination (non-visible light) to the optical distance sensor camera 424 and to direct incident light illumination (e.g., visible light) to the multichromatic camera. However, it is contemplated that in some configurations the optical distance sensor illumination may be visible light having a first predetermined wavelength range (e.g. a wavelength range corresponding to blue light, to red light, etc.). The beam splitter assembly 422, may be a wavelength selective beam splitter configured to direct incident visible light having the first predetermined wavelength range to the optical distance sensor camera 424 while directing incident visible light at a second predetermined wavelength range to the multichromatic camera 426 (which can include video recording capabilities). The second predetermined wavelength range may include all wavelengths of visible light except for the wavelengths in the first predetermined wavelength range.

The beam splitter assembly 422 may be any beam splitter assembly 422 that is suitable. The beam splitter assembly 422 may include a dichroic beam splitter arranged to relay the first predetermined wavelength range to the optical distance sensor camera 424 and the second predetermined wavelength range to the multichromatic camera.

The beam splitter assembly 422 may also comprise a beam splitter operable to split all wavelengths of the light from the optical imaging device, such as the endoscope, to direct all wavelengths towards both an optical distance sensor 450 and the multichromatic camera 426. For instance, the beam splitter assembly 422 may direct 50% (or any other suitable percent) of the intensity of the light from an optical imaging device, such as an endoscope, toward the multichromatic camera, and 50% (or any other suitable percent) of the intensity of the light from the endoscope toward the optical distance sensor camera 424. A bandpass filter can be provided that is configured to preferentially pass light in the first predetermined wavelength range. The bandpass filter may be disposed between the optical distance sensor 450 of the optical distance sensor camera 424 and the beam splitter assembly 422 in order to block light outside of the first predetermined wavelength range from reaching the optical distance sensor 450. A notched filter configured to preferentially pass light in the second predetermined wavelength range and be disposed between the sensor of the multichromatic camera 426 and the beam splitter assembly 422 to filter the incident light relayed toward the optical distance sensor 450.

The optical distance sensor camera 424 and the multichromatic camera 426 have respective optical elements (e.g. camera lenses and focus controls) located in an optical path between the first housing coupler 470 and the sensors for each of the respective cameras as part of an optical imaging system. Using an endoscope 310 as an exemplar optical imaging device, as illustrated in FIGS. 3A-5 the endoscope includes an endoscope rod-lens receiving path 311, but any type of endoscope capable of transmitting optical distance sensor and visible image data to the respective cameras can be used in endoscopic assemblies of some embodiments (e.g. a flexible fiber-optic endoscope). The endoscopic assembly can also include wireless or wired connections to a data processing unit (a processing unit having at least one processor), and in some embodiments the endoscopic assembly includes a battery to function as a power supply that can include providing power for wireless connections with the processing unit.

The endoscopic assembly further comprises one or more fiducial marker (a fiducial marker array 451) or a fiducial marker array mount 452 configured for attaching a fiducial marker array 451 to a depth sensing module housing, the position of which can be optically tracked to enable registration of the spatial position and orientation of the endoscopic assembly to a coordinate system defined by a preoperative scan of the patient.

An optical distance sensor camera 424 is part of a depth sensing module having a housing containing a beam splitter assembly 422. The optical distance sensor camera 424, has an optical distance sensor camera focus knob 425, and an adjustable focus assembly 427. The optical distance sensor camera 424 can be positioned in an optical distance sensor camera housing that is in communication with an optical distance sensor 450, a fiducial marker array 451 via a fiducial marker array mount 452 and an optical distance sensor wireless processing unit communication module 453.

The depth sensing module 420 can be contained in a depth sensing module housing which has a first housing coupler 470, a second housing coupler 471, and a third housing coupler 472 when the depth sensing module 420 is configured to adapt an existing optical imaging device to provide optical distance sensing functionality. The depth sensing module is configured to be in communication with a distance-sensing illumination source. The depth sensing module can be coupled to the distance-sensing illumination source.

The first housing coupler 470 is operable to mechanically connect the depth sensing module 420 to an endoscope 310, the second housing coupler 471 is operable to mechanically connect the depth sensing module 420 to a multichromatic camera 426, the third housing coupler 472 is operable to mechanically connect the depth sensing module 420 to a while light source 446. Each of the first housing coupler, second housing coupler, and third housing coupler can be formed integrally with a depth sensing module housing and operable to mechanically engage a mechanical interface on the endoscope 310, multichromatic camera 426, and a while light source 446.

A suitable power source can be provided, including a removable battery 480. Output from the depth sensing module 420 can be displayed on suitable display device 390 such as a touch screen display.

In the example of FIG. 5, instead of a light source 446, an off-the-shelf light source 546′ is provided in communication with a bifurcated light guide 547′. The bifurcated light guide 547′ is provided instead of an internal light guide bifurcation. Additionally, a camera control box 540 is connected to the depth sensing module 320 and the processing unit 436. The ToF sensor and visible sensor can, for example, be located in the same adapter.

The depth sensing module may also include removable battery 480 for powering the various components of the depth sensing module 420. At least one fiducial marker array 451 is mounted to the optical distance sensor module. The module housing may be releasably mountable to an endoscope, a multichromatic camera, and/or a light guide. In some embodiments, the depth sensing module includes a first coupler configured to couple the endoscope to the module housing, a second coupler configured to couple the multichromatic camera to the module housing, and/or a third coupler configured to couple the light guide to the module hosing. In some embodiments the first coupling, the second coupling, and/or the third coupling include one or more mechanical elements to prevent rotation of the endoscope, light guide, or multichromatic camera relative to the optical distance sensor camera and/or an illumination source (which may be integrated into the module housing in some embodiments, as shown in FIGS. 3A-6). The depth sensing module may include adjustable focus knobs for the visible light imaging and optical distance sensor camera in some cases.

The depth sensing module may include an optical distance sensor wireless processing unit communication module to allow communication between the depth sensing module and a remote processing unit including at least one processor (e.g. to allow for transmission of optical distance sensor camera data). In some embodiments, the communication module, e.g., the processing unit wireless communication module or the optical distance sensor wireless processing unit communication module, is a wired communication module. The processor(s) may be configured to receive the optical distance sensor camera data and to compute a point cloud based on the data.

Further, embodiments disclosed herein may provide markerless, image-based optical navigation for performing procedures. Markerless navigation refers to navigation performed without requiring implanting or otherwise positioning a reference marker at landmark. Navigation methods according to embodiments disclosed herein may provide the three-dimensional (3D) registration of a device (e.g., surgical navigation device or borescope) to a coordinate system established from a 3D scan of the target location, the registration enabled by acquiring real-time depth maps using either a LiDAR camera that acquires real-time depth data via optical TOF measurements, or a visible light imaging-to-depth deep learning algorithm trained on data obtained from a LiDAR camera.

Optical distance sensing-based technology, such as LiDAR technology, as disclosed herein can provide full-field depth maps of a target location in real time through conventional visible light imaging devices, thereby enabling practical markerless registration during a procedure. By extension, it also provides a basis of deep-learning-based depth measurement, enabling the same markerless registration. Some embodiments disclosed herein therefore may include: (a) a LiDAR based device configured to collect accurate depth information through conventional devices; (b) an image-guided navigation using real-time LiDAR-based markerless registration; and (c) methods for applying or displaying information obtained utilizing LiDAR-based 3D tissue measurement.

An assembly is an integrated or modular instrument comprising a LiDAR camera and a multichromatic camera functionally coupled to a device (e.g., surgical navigation device or borescope), and further comprising any connectors, adapters and additional components required to enable a navigation system according to embodiments disclosed herein.

It should be noted that the optical distance sensor camera disclosed herein may be substituted for any suitable time of flight camera, as the disclosure is not so limited. Similarly, the multichromatic camera disclosed herein may be substituted for any suitable imaging camera, as the disclosure is not so limited. Cameras may include various optical components, such as sensors, lenses, and other components for capturing image data.

Light guide: A light guide is included and used to couple both light and optical distance sensor illumination to the endoscope's fiber optic transmission system.

Fiducial marker arrays: A plurality of fiducial marker arrays, e.g. fiducial marker arrays 451, can be provided for each procedure, including, for example, a scope marker fiducial array, an instrument marker fiducial array, and a calibration target fiducial array. The scope marker fiducial array mounts on the optical imaging device (e.g., endoscope), camera, or optical distance sensor based module; the instrument marker array mounts onto the specific navigated instrument. The calibration marker array contains one or more divots or insertion points and can be used for performing a marker-to-tip calibration for the scope and instrument.

Instrument tracker: An instrument tracker 850 situated on a rolling cart tracks the position of the scope fiducial markers and potentially of any other navigated instrument. On the back end, the instrument tracker 850 localizes all instruments to the scope's reference frame.

The navigation system is based on real-time surface registration between an intraoperative 3D image of the intracorporeal space obtained through the endoscope and a corresponding surface identified in the preoperative scan. The intraoperative 3D image is obtained using an optical distance sensor module compatible with a standard endoscope, and the scan surface is identified through preoperative scan segmentation and processing. The output of the 3D registration is a real-time coordinate transformation of the 3D scan to the endoscope's coordinate system.

Simultaneously with this 3D registration process, the instrument tracker 850 tracks the position of the endoscope and navigated instrument using mounted fiducial marker arrays 451, 451′, 451″, and uses this information to localize the instrument in the reference frame of the camera. This combination of 3D measurement, registration, and instrument tracking ensures that the navigation system correctly estimates the relative position between the navigated instrument and patient anatomy, thereby enabling it to provide the user with an accurate navigation signal.

### Calibration

In some embodiments, the target portion may include at least three target planes disposed transverse to each other, each target plane including one or more target(s). However it is contemplated that any suitable number of target planes may be used, as the disclosure is not so limited.

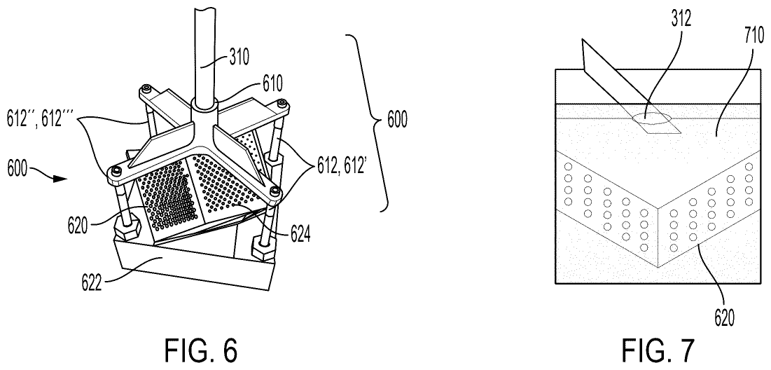

Calibrating the devices and systems may include a calibration fixture 600 with a holder 610 as shown in FIG. 6 that is oriented to direct a tip of the optical imaging device, such as an endoscope 310, towards an intersection point of the at least three target planes defined by a cubicle body 622. A holder 610 is provided to securely position the endoscope 310 relative to the target planes defined by the cubicle body 622 and the target portion 620. Spacers 612 are provided to separate the target planes of the cubicle body 622 from the target portion 620.

The target(s) in each target plane defined by the cubicle body 622 may be any suitable targets. For instance, the targets 624 may be a dot grid of reflective dots. In some instances, the reflective dots may be disposed on a high contrast background (e.g. a black or other dark color background). In some embodiments the size and shape of the dots in each dot grid may be non-uniform. In some cases, the size of the dots may increase and/or the spacing between the dots may decrease the closer a dot in the respective dot grid is to an intersection point of all of the target planes. However, it is contemplated that any suitable style of targets (e.g. as checkerboards, Charuco calibration boards, etc.) may be used, as the disclosure is not so limited.

In some embodiments, the calibration fixture 600 may include a housing configured to hold the target portion, the scope tip, the holder portion, and a fluid medium (e.g. water, saline, etc.). A cubicle body 622 can be provided with apertures on the sides. The cubicle body 622 has at least four sides. An opening may be provided in lieu of top or bottom sides. The cubicle body 622 is secured to the holder 610 and is angularly positioned relative to an axis created by the endoscope 310 tip. Each side of the cubicle body 622 is positioned in a plane that is perpendicular to a plane of an adjacent wall of the cubicle body 622 and is perpendicular to a plane (or imaginary plane) corresponding to the top side and the bottom side.

As seen in FIG. 7, the target portion 620 and the scope tip 312 may be immersed in a fluid solution 710, such as a saline solution. This arrangement may allow the calibration procedures to account for light distortion and travel time through that fluid medium. The fluid medium used for calibration may be the same type of fluid medium present in the subject during the planned operation. For instance, if an operation uses a particular saline solution, that particular saline solution may be poured into the container to ensure that the calibration operation(s) account for the distortion of light as the light passes through that particular saline solution. As will be appreciated by those skilled in the art, saline causes optical distortion, speed of light travel (thus creating an offset in depth sensing/time of flight technology), and attenuation, all of which we can model.

Alternatively, if the fluid medium for an operation is air (e.g. laparoscopy), the container is not filled with a liquid or target portion and holder portion are not placed in a container. As a result, the light will also pass through air during calibration, ensuring that the fluid medium for calibration is the same as the fluid medium during the operation.

As noted above, the calibration fixture may be used for various different calibration procedures. Examples of such calibration procedures include focus calibration, optical distance sensor depth calibration, intrinsic optical distance sensor calibration, and camera alignment.

Focus calibration may be performed by manually or automatically (e.g. algorithmically) setting a focus of the optical distance sensor camera 324, the multichromatic camera, or any other camera used in the endoscopic assembly to maximize a sharpness of the target(s) on the target portion.

LiDAR depth calibration may involve using the known distance of the target(s) from the tip of the optical imaging device when the optical imaging device is in the holder portion and the amount of time it takes for light to travel from the target to the optical distance sensor camera 324 to compute a timing offset. This timing offset may then be used to calculate the distances between the tip of the endoscope and other objects during device use. Depth calibration may also involve measuring a temperature of a laser which services as the illumination source for the optical distance sensor camera 324, as such a temperature may effect timing of the light reaching the optical distance sensor camera 324, and thus effect the depth calibration. In some configuration, the system measures the temperature of such a laser using any suitable sensor at a given depth and uses any suitable means to adjust the measurement dynamically based on the measured laser temperature.

For example, LiDAR intrinsic calibration may involve using the known distances and orientations of the target(s) relative to the scope (e.g. the targets' coordinates in any suitable coordinate system relative to the tip of the scope) to compute a set of anti-distortion parameters (e.g. principal point, focal length, and skew). These anti-distortion parameters may then be used to correct for distortion in the endoscope assembly, ensuring proper calculation of coordinates of a point in the point cloud forming the depth map relative to the scope tip.

Camera alignment may be used in embodiments where the endoscopic assembly includes more than one camera (e.g. a LiDAR camera and a multichromatic camera). In such an arrangement, an image from each camera may be taken, and a transformation to the images may be calculated and applied to the camera(s) to ensure that all targets appear in the same location for each image.

Systems and methods for using an optical distance sensor-type adapter as part of a surgical navigation system for endoscopic procedures by enabling viewing and TOF measurements through a single endoscope. Further, the use of the adapter as part of a navigation system requires the 3D reconstruction obtained to be accurate enough for live 3D registration of the anatomical scan to the visualized anatomy, and this accuracy, particularly in the modular form which can attach to different scopes, is uniquely enabled by the calibration procedures (acquisition parameters, depth, distortion, and camera intrinsic parameters) described in the present disclosure.

FIG. 9 illustrates an exemplary process flow 900 schematic for surgical navigation associated with a surgical procedure, using a navigation system to generate and display navigation signals using data from a three-dimensional preoperative scan such as an MRI or CT scan of a portion of a patient's anatomy. The process flow 900 has inputs, processing and outputs. The inputs, processing and outputs are further divided into preoperative and intraoperative phases.

Referring to the FIG. 9 labelled Preoperative, intraoperative, inputs, processing and output. The digital data from a preoperative scan 910 is uploaded 912 to the navigation system and segmented 914, that is, partitioned into discrete groups of pixels (image segments) to inform object detection at a surgical location in a patient's body, a 3D coordinate system being defined by the segmented data. In some embodiments, the segmentation is then refined manually 916. The refinement and segmentation in some embodiments comprise an iterative process. A surgeon then prepares a surgical plan 918 based on the segmented scan data, including but not limited to determining specific surgical instruments to use during the surgical procedure, and preferred access paths to surgical targets in the patient's body.

Moving to the intraoperative portion 920, an optical imaging module 930 according to disclosed embodiments can include an optical distance sensor module (including the optical distance sensor camera 324 and associated coupling devices) that is calibrated to enable depth data acquired using the optical distance sensor camera 324 to be used to register a field of view of the optical distance sensor assembly to the 3D coordinate system. In one embodiment, optical distance sensor acquisition parameters are utilized to acquire a depth map 932 which is rectified 934 utilizing depth rectification parameters. Distortion is corrected 936 utilizing camera distortion parameters, deprojection 938 is accomplished using camera intrinsic parameters, and 3D registration is generated 940.

Then an instrument tracker 950 is used to identify the marker positions 952 and orientations of the endoscopic assembly and of one or more surgical instrument to be used during the surgical procedure, by identifying marker positions, tracking respective fiducial markers mounted thereto or integrated therewith, computing instrument positions 954 and orientations from the tracking data utilizing marker-to-tip calibrations in this construction, and transforming the data into the 3D coordinate system 956. Finally, data from the multichromatic camera is transformed into the 3D coordinate system to enable real-time display on a display device of a live video signal in the 3D coordinate system by generating navigation signals 958 that are displayed on a display device 970.

Depth information obtained intraoperatively using optical distance sensor enables the acquisition of intraoperative measurements that include but are not limited to proximity of an endoscope to a tissue surface, point-to-point distances between landmarks, distance over a surface, surface area, volume, quantitative distance and angle based correction of fluorescent signals, image augmentations including graduated grid overlays and artificial shadows, and the ability to display the surgical scene from alternative perspectives. In an embodiment, a point of view for observing a surgical procedure is selectable by the surgeon performing the procedure. In a further embodiment the point of view is modifiable by the surgeon during the procedure. In another embodiment the perspective is selectable separately to be different for different observers of the procedure. The surface area (also called intrinsic area or curvilinear area) is an area of the region lying on an actual 3D surface, respecting curvature. The surface area is the area obtained by integrating over the portion of the actual 3D surface that lies inside the user-drawn contour. This area follows the curvature of the underlying surface and corresponds to the true physical area on the tissue. Thus, the surface area (intrinsic or curvilinear) is the area of the curved piece of tissue itself, not the area of its shadow on a flat plane. The projected area (also called planar area or cross-sectional area) is an area of a contour that encloses in 2D space. This is the area enclosed by the same contour after it is projected onto a plane, typically the best-fit plane or the imaging plane. The projected area represents the planar cross-section that the contour spans, independent of surface curvature.

The surface area differs from the projected area, which ignores curvature and measures the 2D footprint of the contour as if viewed straight down, which results in the projected area underestimating area on a highly curved surface. As will be appreciated by those skilled in the art, the surface area respects the local slope on the tissue, taking into account steep or folded regions that contribute more area than they appear from a simple to-down view.

The multichromatic camera 960 can be used to acquire a visible-light image 952 which is fed into the navigation signal generation 958 process before delivery to the display device 970.

Prior to beginning the navigated task the patient scan is uploaded and the anatomical area of interest is segmented using a combination of manual and automatic methods, and a plan is defined relative to the scan. During the navigated task, information from the optical distance sensor module is acquired using a previously optimized set of acquisition parameters, then corrected for depth-based distortions using the depth rectification (alternatively, “depth calibration”) parameters, then correct for image-based distortions using the camera distortion parameters, then deprojected from a depth map to a point cloud, FIG. 7, using the camera intrinsic parameters.

This point cloud (which could alternatively be further converted to a 3D reconstruction in which all the points are connected into a continuous surface) is fed into a 3D registration step that aligns the segmented scan and associated plan to the point cloud, thereby placing the scan and plan into the common coordinate system. Simultaneously during the navigated task, the marker array positions are obtained from the instrument tracker, and are used to compute the positions of the instruments themselves using the marker-to-tip calibration information, and this is used to transform the instrument position to the common coordinate system (“scope frame”).

Subsequently, the placement of the instrument, scan, and associated plan into the common coordinate system allows the generation of navigation signals, which specifically involves generation of 2D views showing a projection of the instrument position and surgical plan within orthogonal slice planes of the scan simultaneously, thereby conveying to the surgeon the complete 3D information of the instrument position relative to the anatomy of interest and surgical plan. These views are rendered on the display device simultaneously with the visible light imaging view, which has the same field of view as the depth map but just shows the visible light imaging image. In some embodiments, a multi-camera alignment between the multichromatic camera and time of flight camera can also be used to render the plan or other navigation information on the visible light imaging image.

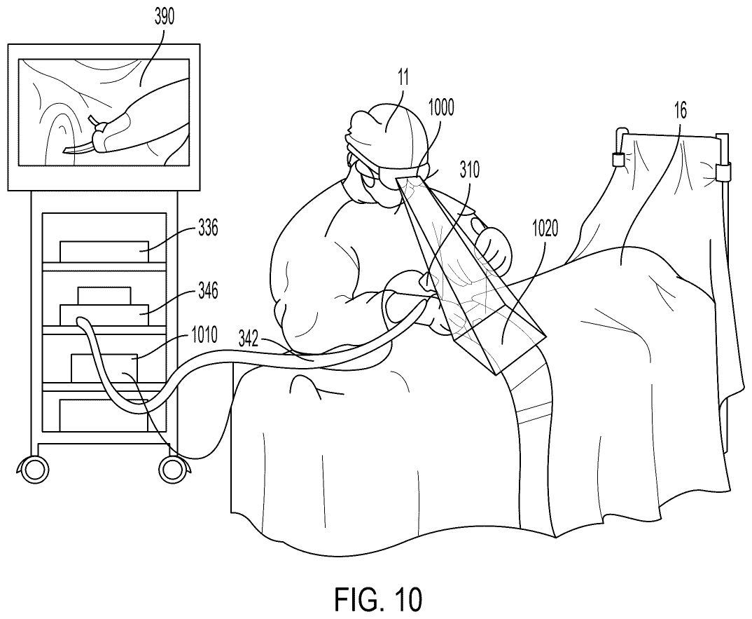

FIG. 10 schematically illustrates a nonlimiting example of a navigation system according to an embodiment, providing an alternative visualization perspective for a surgeon 11 performing a surgical procedure on a patient covered by a drape 16 while using VR 1000 (virtual reality) or AR (augmented reality) glasses or headset to simulate direct visualization (view of surgical scene 1020) of the surgical scene, providing an artificial view of the scene as it might look if the external tissue surface were transparent or if the procedure were being performed open. The processing unit 336, communicates with a light source 346, and video acquisition unit 1010.

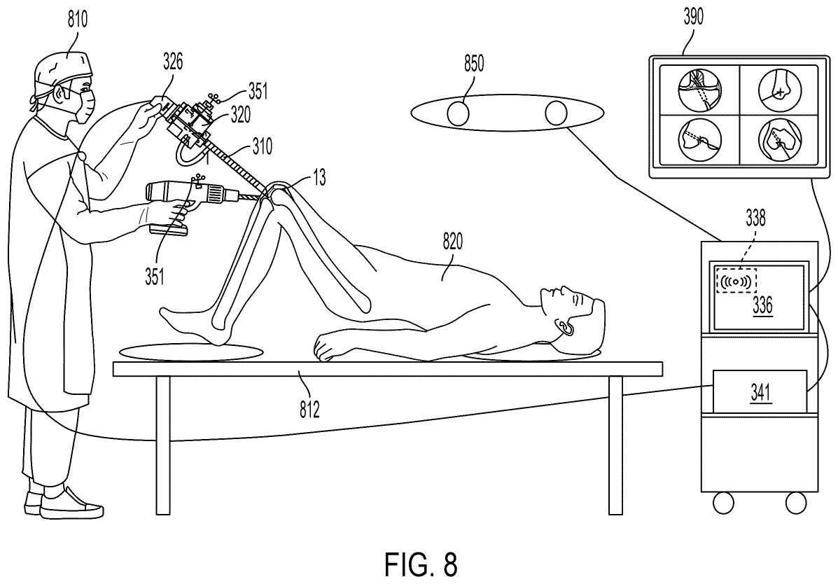

In addition to the tracking functionality of the navigation systems and methods disclosed herein, the navigation system illustrated in FIG. 10 computes a textured 3D reconstruction of the surgical scene using the aligned visible light imaging and depth information obtained from the optical distance sensor module, and then performing a coordinate transformation of the 3D reconstruction to the perspective of the VR/AR glasses, giving the user the impression they are directly visualizing the tissue from their own perspective. In an embodiment, except for the addition of the VR/AR glasses and any support technology required for its operation, the surgical navigation system represented in FIG. 10 functions in the same manner as the navigation system illustrated in FIG. 8.

FIG. 10 depicts a view of a procedure using an endoscope 310 on a patient 820 positioned on a table 812 which incudes an instrument tracker 850, a display device 390, a processing unit 336, and a wireless processing unit communication module 338.

3D information obtained from the endoscopic optical distance sensor-based assembly is conveyed to the processing unit, and simultaneously the fiducial markers on the endoscopic assembly are tracked by the wearable VR/AR headset and used to compute a real-time scope-to headset coordinate transformation. The 3D information from the endoscopic assembly and the scope-to-headset coordinate transformation are simultaneously relayed to the processing unit, which creates a 3D reconstruction from the depth information in the manner described in FIG. 5 and then transforms the 3D reconstruction to the coordinate system of the AR/VR glasses using the previously calculated static scope marker-to-tip calibration and the real-time updating scope to-headset coordinate transformation matrix. In certain embodiments the processing unit may be co-located with the AR/VR headset. The net result of these calculations is a synthetic 3D view of the anatomy rendered from the perspective of the ARNR headset, giving the user the experience of having “see-through” vision of the anatomy beyond the surface of the skin. While this illustration is specifically made for laparoscopic procedures, it can apply in any of the endoscopic domains including but not limited to laparoscopy, arthroscopy, sinuscopy, ureteroscopy, cystoscopy, thoracoscopy, and others.

Navigation systems according some embodiments are uniquely enabled using endoscopic 3D measurement, which enables direct visualization of rigid tissue surfaces. In contrast, an external navigation system that attempts to correlate the external skin surface geometry to the underlying tissue position would likely not be accurate in many cases (for example in the case of joint surgery) due to the nonrigid relationship between the internal structures and surrounding skin. For example, in the major joints, a thick soft tissue layer surrounding the large bones comprising the joint, coupled with its movement due to joint kinematics or muscular contraction, means there is a highly non-rigid relationship between the external and internal anatomy. Further, marker-based registration approaches such as those used in spine surgery would require additional incisions to be made in endoscopic procedures, and have consequently not been adopted.

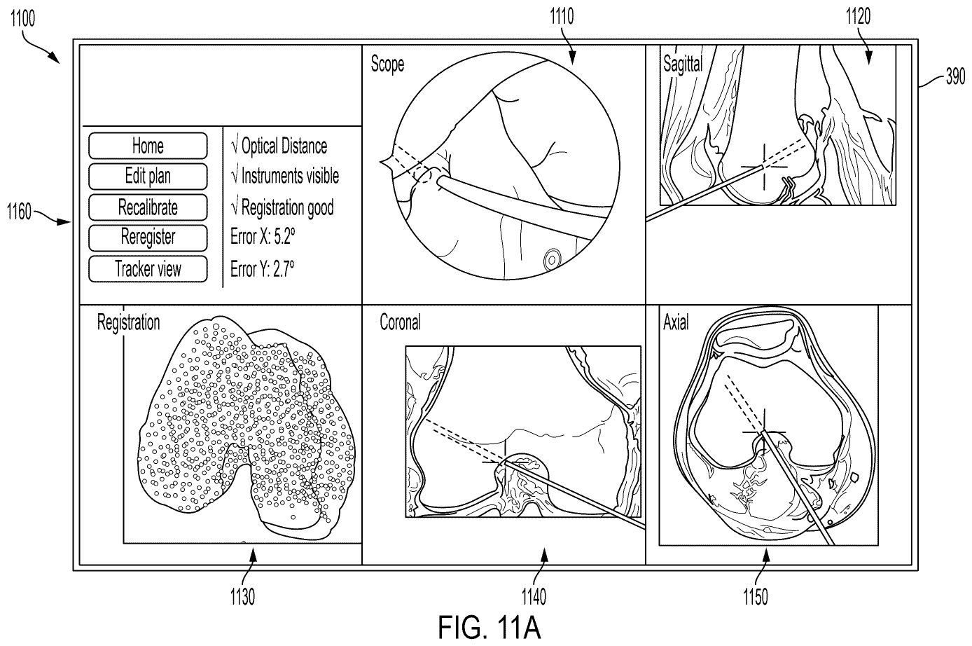

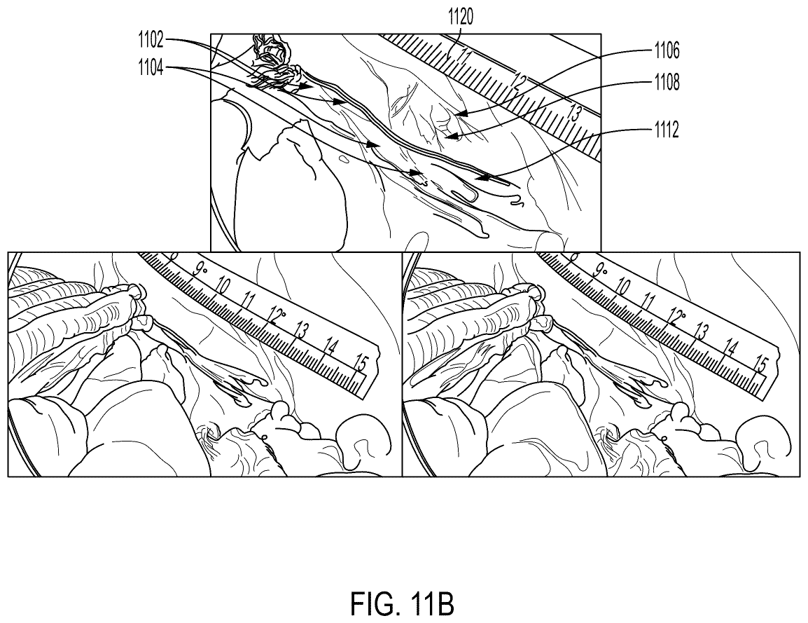

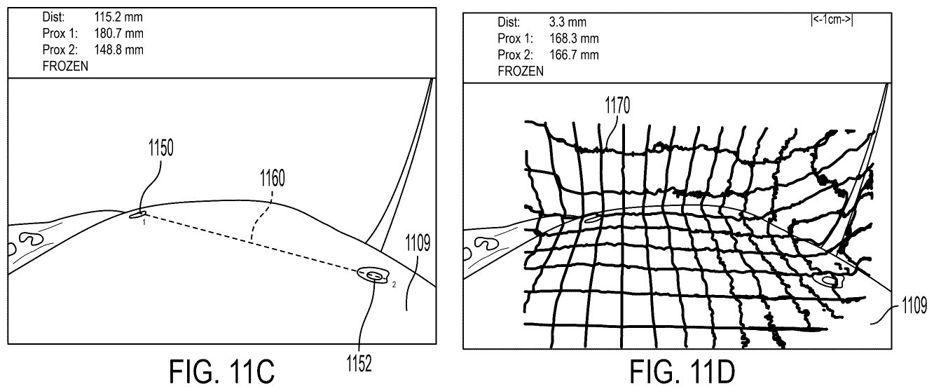

FIG. 11A is a exemplar screen shot 1100 of a user interface of the navigation system illustrating user options including performing calibration again, or editing the surgical plan as well as quantitative navigation errors 1160 (top left), real-time feedback on the registration between the scan and the optical distance sensor-based point cloud 1130 (bottom left), the visible light imaging view from the scope 1110 (top middle), and 2D projections showing the navigated instrument's position relative to the scan and surgical plan, with the projection planes being aligned to the cardinal anatomical axes as defined in the scan's coordinate system 1140, 1120, 2250 (bottom middle, top right, and bottom right as “Coronal”, “Sagittal” and “Axial” views, respectively).

FIG. 11B illustrates a ureter 1106, aorta 1108, peri-lymphatic tissue with underlying aorta 1112, with the para-aortic lymphatic vessels 1102 and the para-aortic lymph nodes 1104 marked. One or more of a distance measurement device 1120 and an angular measurement device 1122, may be provided or overlaid on the image to provide geometric context for the visualized tissue.

FIGS. 11C and 11D illustrate a target surface 1109 with a first point 1150 selected and a second point 1152 selected with a line 1160 superimposed between the two points. A header can be provided in the image that conveys data to the user such as distance, three-dimensional component distances, size, etc. In FIG. 11D, a mesh overlay 1170 can be depicted to provide three-dimensional context to the image for the user, such as a plurality of sets of points, each of the plurality of sets of points comprising points that are equidistant from a reference point associated with a tip of the endoscope along at least one spatial or angular component.

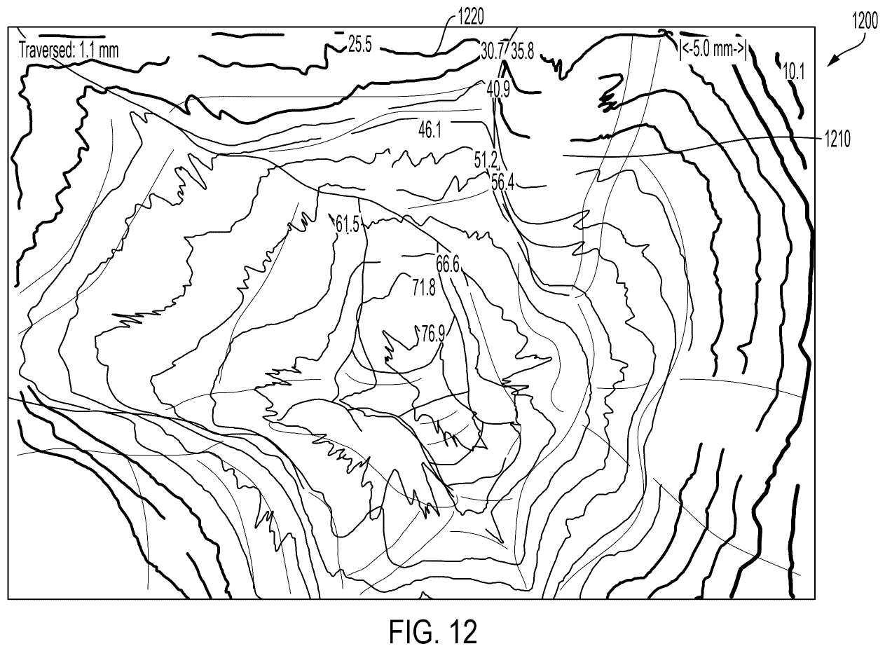

FIG. 12 is a screen shot of a user interface 1200 that shows contour lines 1220 on a target surface 1210 as an overlay and traversal distance for a scope inserted into a lumen.

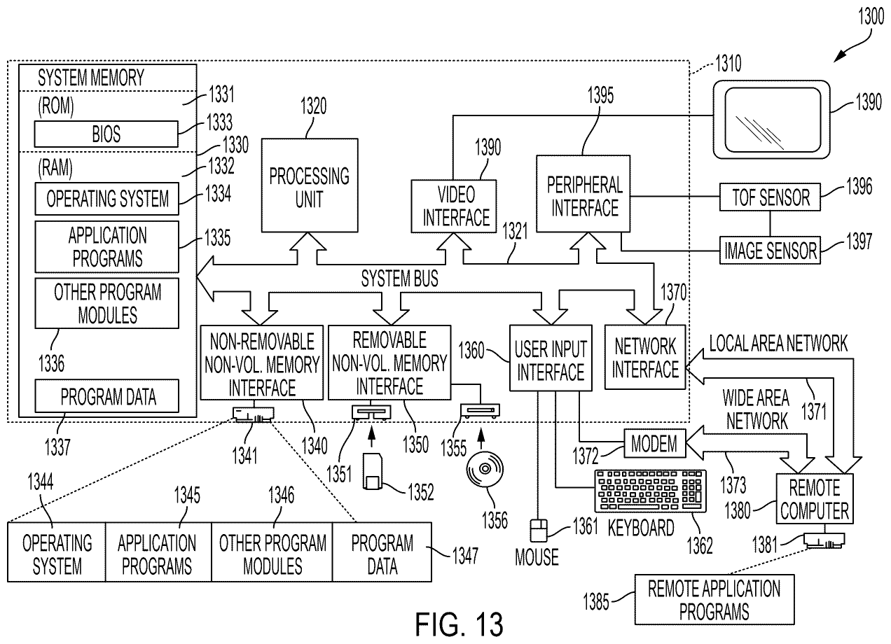

FIG. 13 is a schematic representation of a computer system for use with embodiments disclosed herein;

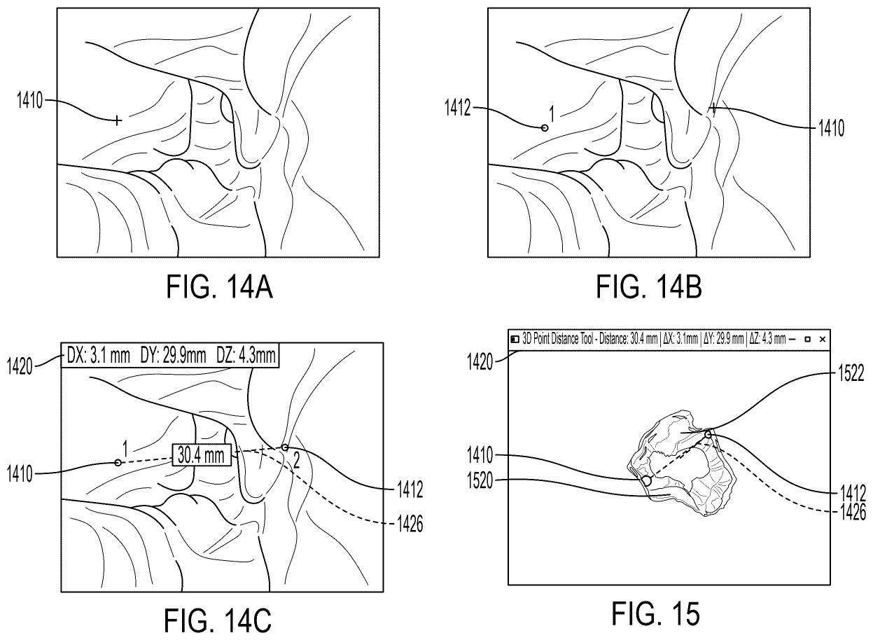

FIGS. 14A-14C are screen shots of a process of using a user interface of a depth measurement system to obtain dimensional information. A curser is positioned at a starting point 1410, which becomes the first point 1412. A second point 1414, located away from the starting point, is marked. A distance is determined between the first point 1412 and the second point 1414. A distance line 1426 can be superimposed on the image depicted on the user interface. A header can be provided on the image depicted on the user interface that provides data, such as distance, coordinate location, etc.

FIG. 15 is a screenshot of a visual representation of 3D information generated by a depth measurement system separate from an image captured by the depth measurement system. A coordinate reference 1522 can be provided on the image.

### III. Surgical Methods

Other embodiments will occur to those skilled in the art and are within the following outline of an arthroscopic procedure according to some embodiments.

### Preoperative Workflow

(Clinical) A diagnostic scan is taken of the knee, a torn ACL is identified, and ACL surgery is prescribed.

### (System) Scan Preparation Step

(UI) System is turned on.

(UI) Patient MRI scan is uploaded through either USB or PACS system.

(UI) The patient's femur surface is segmented from the scan using any combination of automatic and manual methods. The surface segmentation can either include the intact condylar cartilage and the bone, or the bone only, depending on what the surgeon expects to see during the procedure.

(UI) The patient's tibial surface is segmented from the scan.

(UI) User proceeds to procedural planning.

### (System) Procedural Planning Step

(UI) The user plans a femoral tunnel by defining a trajectory and associated thickness on the segmented femur.

(UI) The user plans a tibial tunnel by defining a trajectory and associated thickness on the segmented tibia.

(UI) The user chooses the instruments they will use to place the tunnels (likely a drill or K-wire)

(UI) User proceeds to optical distance sensor system setup or saves session for loading during the procedure.

### Intraoperative Workflow

(Clinical) Initial non-navigated procedural steps are performed. These include performing the Lachman test, administration of anesthesia, patient positioning, sterile preparation, initial arthroscopic access and exam, harvesting and preparation of the ligament graft, and arthroscopic clearance of the tom ACL and other residual soft tissue.

### (System) Lidar System Setup Step

Optical distance module is attached to the scope and camera, battery is inserted, and module is turned on and paired to processing unit.

Light guide is connected to optical distance sensor module illumination source, light source, and scope.

(UI) Optical distance module is calibrated for intrinsic camera parameters using the optical distance sensor camera 324 calibration target.

(UI) User proceeds to tracker system setup.

### (System) Tracker System Setup Step

(UI) Instrument tracker is positioned so that the UI shows it has a clear field of view of the knee and surrounding area.

Lidar marker array is attached to optical distance sensor module.

Instrument marker array is attached to instrument.

(UI) Lidar/scope marker to tip calibration is performed using the calibration marker array.

(UI) Scope marker to tip calibration is performed using the calibration marker array.

(UI) User proceeds to femoral registration.

### (System) Femoral Registration Step

(UI) Scope is inserted and intercondylar notch is visualized.

11. (UI) Initial registration is performed with the MRI intercondylar notch surface.

111. (UI) User proceeds to femoral tunnel navigation.

### (System) Femoral Tunnel Navigation Step

(UI) Surgical scan data and femoral tunnel trajectory is visualized.

(UI) User inserts instrument (guide pin or drill) into incision and instrument position is shown relative to scan and planned trajectory. Metrics are displayed. showing instrument correspondence with planned trajectory.

(UI) User drills femoral tunnel when ready.

1v. (UI) User proceeds to tibial registration.

### (System) Tibial Registration Step

(UI) Scope is inserted and intercondylar area is visualized.

(UI) Initial registration is performed with the MRI intercondylar area surface.

(UI) User proceeds to tibial tunnel navigation.

### (System) Tibial Tunnel Navigation Step

(UI) Surgical scan data and tibial tunnel trajectory is visualized

(UI) User inserts instrument (guide pin or drill) into incision and instrument position is shown relative to scan and planned trajectory. Metrics are displayed showing instrument correspondence with planned trajectory

(UI) User drills tibial tunnel when ready

1v. (UI) User shuts system off.

(Clinical) The rest of the procedure is completed, including tunnel tensioning, fixation, biomechanical evaluation, and incision closure

In some embodiments, it may be desirable to provide a user with dimensional information incorporated into a visual representation (e.g. a live video feed) of an object. Such an arrangement may allow a user to easily and accurately assess and/or measure various dimensional characteristics of the object. For instance, FIG. 8 illustrates a visual representation of the inside of a lumen captured by an endoscopic assembly in which rings are artificially overlaid on the walls of the lumen. In FIG. 8, each ring is overlaid on points that are a given distance away from the scope tip. Other dimensional information may be incorporated or displayed alongside the visual representation as well. For instance, an indicator on the top right of the screen indicates the separation distance between successive rings, and measurement included on the rings themselves indicate the average distance of all the points in the ring from the tip of the endoscope. Such rings may allow a user to easily assess locations of the endoscope and various parts of the lumen relative to each other, as well as sized of various features of the lumen.

An additional indicator can be provided on the top left of the screen may be included which indicates an estimate as to the distance the scope has traversed through the lumen. The user may have the option to zero this estimate at any time in order to compute a traversal distance. Such an arrangement could be useful in clearly defining the position of a target, such as a tumor or a stricture, along the length of the lumen. A similar overlay could also be generated for the radial position of the target, and this information could be combined with accelerometers built into the endoscope assembly to better describe the radial position of surgical targets with respect to gravity, or to the patient's cardinal anatomic planes.

Thus, in some embodiments, a depth measurement system may be provided. The depth measurement system may include an endoscope assembly which includes at least an endoscope and a time of flight (TOP) sensor (or other 3D measurement sensor). The endoscope assembly may be arranged according to any of the embodiments of an endoscope assembly disclosed herein, or any other suitable endoscope assembly. In operation, the endoscope assembly may be inserted into a desired area (e.g. the lumen 1220 with a lumen surface 1210 shown in FIG. 12), and the TOP sensor may generate a plurality of images and transmit the images to a processing unit with one or more processors. In some embodiments, the endoscope also includes an image sensor (e.g. a multichromatic sensor), which transmits a plurality of images to the processing unit. An overlay of contour lines 1200 are provided to show topography of the interior lumen surface.

The processor(s) may then compute a point cloud based on at least some of the images output by the TOP sensor, and render a visual representation of an object which the endoscope is pointed at (e.g. the lumen of FIG. 8). Such rendering may involve displaying the visual representation on a screen. The visual representation may be any suitable visual representation of the object. For instance, the visual representation may be an image obtained by the TOP sensor or, if included, the image sensor. (Such an image sensor may be aligned with the TOP sensor using the calibration procedures described herein, or any other suitable calibration procedures.) The processor(s) may then register 3D information computed based on the point cloud with the visual representation. This 3D information may be the point cloud itself, a 3D mesh, or any other suitable 3D information. The processor(s) may then receive user input indicating a location in the rendered visible representation and compute a dimensional parameter of the indicated location using the 3D information. Such an arrangement may allow, for example, for a user to select points on a displayed image of the object, and measure a distance between these points. For instance, as seen in FIGS. 14A-14C, a user may place a cursor 1410 at a first location on the object in the image (FIG. 14A). The user may then select the first location to establish the first location as a first point, and then move the cursor to a second location 1412 on the object (FIG. 14B). The user may then select the second location to establish a second point, and the processor(s) may compute various dimensional information about the locations of the first and second points, for example, distance 1426 between 1410, 1412. For instance, as seen in FIG. 14C, the processor(s) may compute a straight line distance 1426 between the points (1410, 1412), as well as distance between the points in a preestablished coordinate system (see e.g. FIG. 15). It should be appreciated however that the system may alternatively or additionally compute other dimensional information, such as the diameter of the lumen in a plane through the designated points. In some embodiments, the processor(s) may compute a longitudinal measurement of the length of the lumen between the first point and the second point. In some embodiments, the user may be able to select additional points, and the processor may compute additional parameters based on the locations of the points (e.g. surface area and/or volume within a boundary defined by the points). Dimensional data 1420 can be shown on the image.

In some embodiments, the processor(s) may generate a visual representation of the 3D information separate from the depth image. For instance, as seen in FIG. 15, the processor may generate a mesh 1520 characterizing an inner surface of a lumen. In some embodiments, one or more measurement processes discussed above in relation to FIGS. 14A-14C may be performed on this separately generated visual representation.

Alternatively, the processor(s) may automatically compute one or more dimensional parameters of an object which the endoscope is directed at. When the object is a lumen, the processor(s) may compute a diameter, circumference, surfaces area, volume, and/or other parameter of the lumen (either automatically or in response to an indication by the user). In some cases, the user may indicate a specific location along the length of the lumen, and the processor(s) may compute the desired parameter(s) of the lumen at that indicated location.

In some embodiments, as seen in FIG. 12, the 3D information is an image overlay comprising a plurality of lines, each line being formed of points of equal distance from the tip of the endoscope. The processor(s) may be configured to generate the plurality of lines and render the visible representation of the object with the image overlay superimposed on the visible representation. Such an arrangement may allow for the user to see the plurality of lines on the displayed image.

As noted above, it may be desirable to allow a user to be able to measure/monitor a traversal distance of the endoscope along the lumen. The processors may therefore be configured to receive the user input indicating a first time and a second time and compute a traversed distance along the lumen between a location of the tip of the endoscope at the first time and at the second time.

The processor may also be configured to indicate a characteristic of the lumen in the visual representation at a segment of the lumen based on an indication of the characteristic and the segment. Such a characteristic may be, among other characteristics, a stricture and/or variation in diameter of the lumen.

### IV. Industrial Methods

A borescope process is a remote visual inspection performed by a technician using a thin probe with an integrated camera and light source to examine otherwise inaccessible internal surfaces (e.g., engine cylinders, pipes, turbines). The probe is inserted through an access port, guided under visual control to the target area, and images or video are captured for documentation and later review.

Initially the technician prepares and makes the environment to be inspected safe. Preliminarily, de-energize and cool the equipment, establish lockout/tagout as needed, and select and check the appropriate borescope and optics for the access and inspection task. Access to the area to be inspected is obtained and a borescope is inserted. Access can be achieved by, for example opening or removing access plugs/covers, then gently insert the borescope probe through the selected port, avoiding contact damage to internal components or the probe.

Once access is achieved, the borescope is steered or articulated to systematically survey the internal surfaces (e.g., cylinder walls, valves, welds), viewing the live image on a monitor and adjusting focus and illumination as needed. Positioning an optical distance sensor in addition to a monochromatic sensor (either as an adapter or integrated into the system) allows the borescope to provide three-dimensional imaging data along with two-dimensional data as described above.

The system is configurable to capture still 2 dimensional images and/or video of relevant features such as cracks, corrosion, deposits, or wear patterns, and record their locations for maintenance planning or compliance records. Additionally, three-dimensional images and videos can be provided. Once the evaluation is complete, the borescope probe is withdrawn, cleaned and checked, then close and secure access ports and return the inspected system to its normal configuration.

### V. Computing Systems

FIG. 13 illustrates an example of a suitable computing system environment 1300 on which the disclosed methods may be implemented, and may, for example, represent a processor as described herein. The computing system environment 1300 is only one example of a suitable computing environment and is not intended to suggest any limitation as to the scope of use or functionality of the disclosed methods. Neither should the computing system environment 1300 be interpreted as having any dependency or requirement relating to any one or combination of components illustrated in the exemplary computing system environment 1300.

The disclosure is operational with numerous other special purpose computing system environments or configurations, which, in some embodiments, may be created by programming a general purpose computing device 1310 (e.g., computer). Examples of well known computing systems, environments, and/or configurations that may be suitable for use include, but are not limited to, personal computers, server computers (including those implementing cloud computing or data services), smartphones, tablets, hand-held or laptop devices, multiprocessor systems, microprocessor-based systems, set top boxes, programmable consumer electronics, network PCs, minicomputers, mainframe computers, distributed computing environments that include any of the above systems or devices, and the like. Some of the elements illustrated in FIG. 13 may not be present, depending on the specific type of computing device. Alternatively, additional elements may be present in some implementations.

The computing environment may execute computer-executable instructions, such as program modules. Generally, program modules include routines, programs, objects, components, data structures, etc. that perform particular tasks or implement particular abstract data types. Some embodiments may also be practiced in distributed computing environments where tasks are performed by remote processing devices that are linked through a communications network. These distributed systems may be what are known as enterprise computing systems or, in some embodiments, may be “cloud” computing systems. In a distributed computing environment, program modules may be located in both local and/or remote computer storage media including memory storage devices.

With reference to FIG. 13, an exemplary system includes a general purpose computing device in the form of a computing device 1310. Components of the computing device 1310 may include, but are not limited to, a processing unit 1320, a system memory 1330, and a system bus 1321 that couples various system components including the system memory to the processing unit 1320. The system bus 1321 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

Computing devices 1310 typically includes a variety of computer readable media. Computer readable media can be any available media that can be accessed by computing device 1310 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM 1332, ROM 1331, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computing device 1310.

Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer readable media.

The system memory 1330 includes computer storage media in the form of volatile and/or nonvolatile memory such as read only memory (ROM) 1331 and random access memory (RAM) 1332. A basic input/output system 1333 (BIOS), containing the basic routines that help to transfer information between elements within computing device 1310, such as during start-up, may be stored in ROM 1331. RAM 1332 may contain data and/or program modules that are immediately accessible to and/or presently being operated on by processing unit 1320. By way of example, and not limitation, FIG. 13 illustrates operating system 1334, application programs 1335, other program modules 1336, and program data 1337.

The computing device 1310 may also include other removable/non-removable, volatile/nonvolatile computer storage media. By way of example only, FIG. 13 illustrates a hard disk drive 1341 that reads from or writes to non-removable, nonvolatile magnetic media. Such a hard disk drive may be implemented by a rotating disk drive or as a solid state drive, such as is implemented with FLASH memory. FIG. 13 also illustrates a slot 1351 that reads from or writes to a removable, nonvolatile memory 1352, such as a memory stick or FLASH memory, and an optical disk drive 1355 that reads from or writes to a removable, nonvolatile optical disk 1356 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 1341 may be connected to the system bus 1321 through a non-removable memory interface such as interface 1340, and slot 1351 and optical disk drive 1355 may be connected to the system bus 1321 by a removable memory interface, such as interface 1350. However, it should be appreciated that, in some embodiments, some or all of the computer readable media available to a device may be accessed over a communication network.

The drives and their associated computer storage media discussed above and illustrated in FIG. 13, provide storage of computer readable instructions, data structures, program modules and other data for the computing device 1310. In FIG. 13, for example, hard disk drive 1341 is illustrated as storing the operating system 1344, application programs 1345, other program modules 1346, and program data 1347. Note that these components can either be the same as or different from operating system 1334, application programs 1335, other program modules 1336, and program data 1337. Operating system 1344, application programs 1345, other program modules 1346, and program data 1347 are given different numbers here to illustrate that, at a minimum, they are different copies.

A computing environment may include one or more input/output devices. Some such input/out devices may provide a user interface. A user may enter commands and information into the computing device 1310 through input devices such as a keyboard 1362 and pointing device 1361, depicted as a mouse. However, other forms of pointing devices may be used, including a trackball, touch pad or touch screen. Other input devices (not shown) may include a microphone, joystick, game pad, satellite dish, scanner, or the like. The microphone, for example, may support voice input, which may be recorded as an audio file or may be translated, such as using speech recognition, to a text format for further processing. These and other input devices are often connected to the processing unit 1320 through a user input interface 1360 that is coupled to the system bus, but may be connected by other interface and bus structures, such as a parallel port, game port or a universal serial bus (USB).

The computing device may include one or more output devices, including an output device that may form a portion of a user interface. A display device 1391 or other type of display device may also connected to the system bus 1321 via an interface, such as a video interface 1390, to form a visual output device. In addition to the display device, computers may also include other peripheral output devices (not shown) such as speakers or a printer, which may be connected through a peripheral interface 1395. The peripheral interface may enable one or more processors, such as those within processing unit 1320 to control and/or receive data from a TOP sensor 1396 and/or an image sensor 1397, such as a multichromatic camera.

The computing device 1310 may operate in a networked environment using logical connections to one or more remote computers, such as a remote computer 1380. The remote computer 1380 may be a personal computer, a server, a router, a network PC, a peer device or other common network node, and typically includes many or all of the elements described above relative to the computing device 1310, although only a memory storage device 1381 has been illustrated in FIG. 13. The logical connections depicted in FIG. 13 include a local area network (LAN) 1371 and a wide area network (WAN) 1373, but may also include other networks. Such networking environments are commonplace in offices, enterprise-wide computer networks, intranets and the Internet. Alternatively or additionally, the WAN may include a cellular network.

When used in a LAN networking environment, the computing device 1310 is connected to the LAN 1371 through a network interface or adapter 1370. When used in a WAN networking environment, the computing device 1310 typically includes a modem 1372 or other means for establishing communications over the WAN 1373, such as the Internet. The modem 1372, which may be internal or external, may be connected to the system bus 1321 via the user input interface 1360, or other appropriate mechanism.

In a networked environment, program modules depicted relative to the computing device 1310, or portions thereof, may be stored in the remote memory storage device. By way of example, and not limitation, FIG. 13 illustrates remote application programs 985 as residing on memory device 1381. It will be appreciated that the network connections shown are exemplary and other means of establishing a communications link between the computers may be used.

Depending on the nature of the computing device, one or more additional elements may be present. For example, a smart phone or other portable electronic device may include a camera, capable of capturing still or video images. In some embodiments, a computing device may include sensors such as a global positioning system (GPS) to sense location and inertial sensors such as a compass, an inclinometer and/o-Ran mobile network architecture, and accelerometer. The operating system may include utilities to control these devices to capture data from them and make it available to applications executing on the computing device.

As another example, in some embodiments, a computing device may include a network interface to implement a personal area network. Such an interface may operate in accordance with any suitable technology, including a Bluetooth, Zigbee or an 802.11 ad hoc mode, for example.

FIG. 6 discussed above shows a perspective view of an embodiment of a calibration fixture which may be used to perform one or more of the various calibration procedures described herein. The calibration fixture may include a holder portion configured to receive the tip of the endoscope, and a target portion which includes one or more targets. The holder portion may be disposed at a predetermined distance from the target(s) on the target portion (e.g. using one or more spacers). Such an arrangement of the holder portion and the target portion allows the tip of the endoscope to be positioned at a known distance and orientation relative to the target(s).

The present disclosure specifically describes an optical distance sensor-based adapter as a modular, standalone device independent of any specific endoscopes, meaning that the same adapter can detachably be used across multiple types of endoscopes. Detachability enables the optical distance sensor module to be used across different scopes of a given type (for example, laparoscopes or arthroscopes), which can present variable visible light imaging images for the same scene due to variability in factors such as field of view, depth of field, lens distortion, or color aberration. This variability is critical for training a generalized AI-based model that can predict 3D information from the visible light imaging scene alone, and is enabled by being able to use the same module across different scopes. Federated or decentralized training can be provided using multi-site surgical/industrial video and depth maps, with governance constraints with ongoing model improvement incorporated into the training.

Different types of scopes utilizable include, but are not limited to: laparoscopes, arthroscopes, thoracoscopes, cystoscopes, sinuscopes, ureteroscopes, and hysteroscopes. This allows the present platform to be applicable across different specialties, allowing the same module to turn any endoscope into a 3D camera and to enable 3D measurement or 3D reconstruction capability in different procedures performed in different parts of the body.

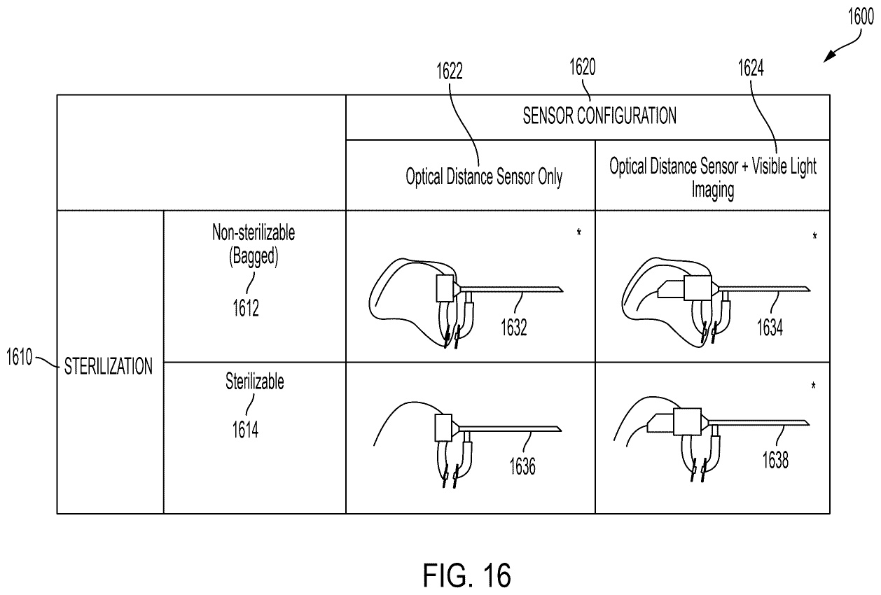

As seen in FIG. 16, detachability may also enable the optical distance sensor module to be sterilized using a different process from the attached scopes. Sensor configuration 1620 can be optical distance sensor only 1622, or optical distance sensor plus visible light imaging 1624. Sterilization 1610, can be non-sterilizable 1612 or sterilizable 1614. The resulting combinations are: (1) optical distance sensor only with bag 1632, (2) optical distance sensor with visible light imaging and bag 1634, (3) optical distance sensor only with sterilization 1636, and (4) optical distance sensor with visible light imaging and sterilization 1638.

Such an arrangement may be desirable as scopes are usually sterilized with steam heat, which can be damaging to electronics, while electronics are sterilized using hydrogen peroxide gas. The scope and optical distance sensor adapter must be detachable to enable them to be sterilized using different processes. Alternatively, detachability enables placement of a transparent sterile barrier between the optical distance sensor module and the scope, enabling the module to be used as a non-sterile device, enabling greater overall device utilization time, which allows higher throughput collection of 3D information for the purpose of training AI models.

The present disclosure describes an optical distance sensor module with a removable fiducial marker mount, such that the module can either be used alone or in conjunction with a navigation system. This is useful because there are various applications that are possible using only the optical distance sensor adapter, including 3D measurement of tissue geometry, normalization of fluorescence intensity based on distance, or creation of synthetic 3D views from alternative perspectives, and separately there are navigation-based applications, namely the planning of surgical targets or trajectories and precise positioning of instruments relative to those surgical plans, enabled through the use of the optical distance sensor system with fiducial marker arrays and navigated instruments as described in this disclosure.

Also described herein is the use of the optical distance sensor-based module along with a wearable headset or 3D display device to create real-time 3D perspectives of the scene, which are only useful with the sub-millimeter accuracy and methods for obtaining such accuracy described here. An example might be the generation of a 3D view of a torn meniscus during knee arthroscopy so as to better assess the damage. Using the system described here, such a capability would be unlocked for standard endoscopes.

This disclosure further describes elements that would be specific to arthroscopic procedures, including:

The use of patient mounted fiducial arrays to on rigid landmarks (such as the patella) to aid with accurate navigation. This is helpful in arthroscopy in particular, in which certain rigid surfaces, such as the patellar tendon, may be accessible when the limb is in certain configurations common for that procedure (for example, the knee is almost always bent in arthroscopic ACL reconstruction), thereby allowing a fiducial marker placed on this rigid surface to have a rigid relationship with the anatomical location of interest (e.g. the patella does not move much relative to the femur, which is where one would be placing a tunnel during ACL reconstruction).

The description of surgical tools that embed fiducial markers, for example K wires or drill-bits that natively have markers presenting optical fiducial patterns, is unique to arthroscopy and would enable a more accurate and streamlined use of the navigation system (streamlined because the surgeon would not have the extra step of attaching the fiducial array to the drill itself and accurate because the fiducials would be closer to the tip of the navigated instrument and thus more accurately represent its position).

The description of a biomechanical analysis which may be a part of the planning step, in which tunnel angles could be determined with biomechanical variables such as mineral bone density, anatomical measurements such as posterior tibial slope, and mechanical simulation of joint movement optimizing to minimize strain or impingement on the graft ligament.

The description of using a “mirror image” obtained by scanning the contralateral limb to position tunnels or fixation points in the affected limb. For example, in ACL reconstruction the literature suggests significant variability in the placement location of the femoral tunnel, even when the footprint of the residual ACL is visible. The position could be determined more consistently if procedural planning involved an automated analysis of the ligament's centerline in a scan of the contralateral femur, followed by determination of the corresponding location on the affected femur.

The techniques described above may be implemented, for example, in hardware, one or more computer programs tangibly stored on one or more computer-readable media, firmware, or any combination thereof. The techniques described above may be implemented in one or more computer programs executing on, or executable by, a programmable computer including any combination of any number of the following: a processor, a storage medium readable and/or writable by the processor (including, for example, volatile and non-volatile memory and/or storage elements), an input device, and an output device. A processor is also referred to herein as a processing resource. The input device and/or the output device form a user interface in some embodiments. Program code may be applied to input entered using the input device to perform the functions described and to generate output using the output device.

Embodiments of the present disclosure include features which are only possible and/or feasible to implement with the use of one or more computers, computer processors, and/or other elements of a computer system. Such features are either impossible or impractical to implement mentally and/or manually. For example, embodiments of the present disclosure automatically generate 3D registration, automatically update data in an electronic memory representing location and movement of an endoscope and/or at least one surgical instrument, and automatically and wirelessly transmit such data to a server over a digital electronic network for storage and processing. Such features can only be performed by computers and other machines and cannot be performed manually or mentally by humans.

Any claims herein which affirmatively require a computer, a processor, a controller, a memory, or similar computer-related elements, are intended to require such elements, and should not be interpreted as if such elements are not present in or required by such claims. Such claims are not intended, and should not be interpreted, to cover methods and/or systems which lack the recited computer-related elements. For example, any method claim herein which recites that the claimed method is performed by a computer, a processor, a controller, a memory, and/or similar computer-related element, is intended to, and should only be interpreted to, encompass methods which are performed by the recited computer-related element(s). Such a method claim should not be interpreted, for example, to encompass a method that is performed mentally or by hand (e.g., using pencil and paper). Similarly, any product claim herein which recites that the claimed product includes a computer, a processor, a memory, and/or similar computer-related element, is intended to, and should only be interpreted to, encompass products which include the recited computer-related element(s). Such a product claim should not be interpreted, for example, to encompass a product that does not include the recited computer-related element(s).

Each computer program within the scope of the claims below may be implemented in any programming language, such as assembly language, machine language, a high-level procedural programming language, or an object-oriented programming language. The programming language may, for example, be a compiled or interpreted programming language.

Each such computer program may be implemented in a computer program product tangibly embodied in a machine-readable storage device for execution by a computer processor. Method steps of the disclosure may be performed by one or more computer processors executing a program tangibly embodied on a computer-readable medium to perform functions of the disclosure by operating on input and generating output. Suitable processors include, by way of example, both general and special purpose microprocessors. Generally, the processor receives (reads) instructions and data from a memory (such as a read-only memory and/or a random access memory) and writes (stores) instructions and data to the memory. Storage devices suitable for tangibly embodying computer program instructions and data include, for example, all forms of non-volatile memory, such as semiconductor memory devices, including EPROM, EEPROM, and flash memory devices; magnetic disks such as internal hard disks and removable disks; magneto-optical disks; and CD-ROMs. Any of the foregoing may be supplemented by, or incorporated in, specially-designed ASICs (application-specific integrated circuits) or FPGAs (Field-Programmable Gate Arrays).

A computer can generally also receive (read) programs and data from, and write (store) programs and data to, a non-transitory computer-readable storage medium such as an internal disk (not shown) or a removable disk or flash memory. These elements will also be found in a conventional desktop or workstation computer as well as other computers suitable for executing computer programs implementing the methods described herein, which may be used in conjunction with any digital print engine or marking engine, display device, or other raster output device capable of producing color or gray scale pixels on paper, film, display screen, or other output medium or other type of user interface. Any data disclosed herein may be implemented, for example, in one or more data structures tangibly stored on a non-transitory computer-readable medium. Embodiments of the disclosure may store such data in such data structure(s) and read such data from such data structure(s).

### VI. Image Guided Prediction and Training Models

For devices and systems that include a TOF camera and a second camera (double camera hardware system), the TOF camera and second camera are configurable to collect a correlated data set. The correlated data set is then provided to a machine learning model with images to train the model, and the output of the model is the correlated and synchronized images from the TOF camera. Movement information can be also be used to further perform imitation learning on human surgeons for the purpose of developing autonomous algorithms.

Upon training, the algorithm is operable to predict the depth data from the second camera alone (single camera hardware system). Once trained, a standard single-camera set up (which is commonly a single RGB camera), can be used to provide the same output as a two camera system thereby enabling the disclosed capabilities and benefits of using a dual camera system to be doable via a software upgrade to an RGB system. The prediction and training models are computer-implemented.

Additionally, a machine learning (ML) based prediction and training model can be combined with known classical depth estimation methods (for example, structure from motion, structure from shadow, fiducial based methods). The outputs of both classical and ML based methods could then be combined across a plurality of pixels using a Kálmán filter, or in another embodiment automatically used one method or the other on a part of the image depending on its characteristics (for example, if the image has lots of features and shadows in it we might get away with the classical method, but if there are lots of obstructions, or the scene is featureless, the deep learning method can be used. As will be appreciated by those skilled in the art, there are many different model architectures that can be employed, including, for example, neural radiance fields or a Gaussian splatting rendering technique.

### A. Surgical

Image-guided prediction and training pipelines can be developed for surgical procedures. Using ACL reconstruction as an example of the process, the information can be framed as a staged, data-governed process: curate and label multimodal data; define prediction and guidance targets; develop and validate models; then integrate them into intraoperative workflows for real-time decision support and training. Preoperative images (CT, MRI, etc.) are processed to build patient-specific 3D models and risk predictions (e.g., surgical complexity, likelihood of complications). During surgery, intraoperative images (fluoroscopy, endoscopy, ultrasound, etc.) are aligned with these models, and AI systems can highlight anatomy, track instruments, or suggest safe paths.

Deep learning models are trained on large sets of labeled preoperative images and outcomes can predict surgical complexity, complication risk, or margin status before a case. Similar models applied to intraoperative images or video can detect structures, estimate margins, and warn when instruments approach critical anatomy.

Recorded surgical video and imaging streams can be annotated into phases, maneuvers, and errors, then used to train models that recognize workflow and technique quality. Models are configurable to power simulators and post-case dashboards that give surgeons objective metrics (e.g., timing of steps, instrument paths, margin distances) and compare performance to expert benchmarks.

In the operating room, outputs can be provided to the surgeon that appear as overlays, color highlights, or simple alerts rather than autonomous commands, keeping the surgeon in control. After surgery, predictions and intraoperative guidance are compared with actual outcomes; the differences are used to refine both the models and the training tools.

### B. Industrial

In industrial settings, image-guided prediction and training models use camera, sensor, and sometimes 3D imaging data to forecast equipment or process problems, guide operators in real time, and build better training systems. The aim is higher uptime, better quality, and safer, more consistent operations.

Fixed and mobile cameras (often with depth, thermal, or hyperspectral imaging) monitor machines, production lines, and assembly tasks to create a visual record of “normal” and “fault” states. Machine-learning models are trained on acquired images learn to recognize defects, misalignments, wear, unsafe conditions, and deviations from standard work.

Condition-monitoring models combine image streams (e.g., surface appearance of parts, flame shape, weld pool geometry, conveyor loading) with process data to predict failures or quality excursions before they occur. Visual anomaly-detection models flag unusual patterns—like subtle surface cracks, incomplete welds, or improper tool positioning—so that operators can intervene early. Video of expert operators and annotated process runs is used to train models that recognize correct versus incorrect motions, sequences, and setups. Models can be optimized to feed into AR/VR simulators and on-line guidance systems that show operators step-by-step tasks, highlight where to place tools or parts, and provide objective performance metrics (time, accuracy, rework).

On the shop floor, model outputs appear as overlays on operator screens, AR glasses, or HMI panels, showing live status, alerts, and recommended adjustments rather than taking direct control. After each shift or batch, detected issues, alarms, and operator actions are compared with production results; this feedback loop refines both predictive models and the training programs used to upskill the workforce.

### EXAMPLES

Techniques as described herein may be embodied in one or more ways, such as:

### A. Endoscope

An endoscopic assembly for obtaining intraoperative, real-time depth information through a standard endoscope using an optical distance sensor module comprising: a housing containing an optical distance sensor camera 324 (such as an electronic optical distance sensor camera) and associated illumination source connected to a source of power; a bifurcated light guide for coupling optical distance sensor illumination and a remote source of light to the endoscope means to releasably couple to the bifurcating light guide such that optical distance sensor illumination can be transmitted to the endoscope; a beam splitting assembly that relays optical distance sensor illumination obtained from the endoscope receive lens to the optical distance sensor camera 324 and light illumination to a multichromatic camera 326 of an endoscope; a coupler to releasably couple a standard endoscope such that illumination obtained through the endoscope transmits through the beam splitting assembly; a coupler releasably couple to a multichromatic camera 326 such that light obtained through the endoscope and transmitted through the beam splitting assembly is captured by the multichromatic camera 326; an adjustable focus mechanism for the optical distance sensor camera 324, an adjustable focus mechanism for the multichromatic camera 326; and a processing unit that acquires the optical distance sensor module outputs which include at least a real time updating depth map and intensity map.

### B. Stapler Systems

Identify a target location for application of a stapler system such as the UroLift®. Prior to using the stapler system, perform diagnostic endoscopy and acquire relevant measurements of anatomic targets including the verumontanum, prostate, capsule, surrounding lumen, total urethra length, prostate urethra length, prostate thickness, estimate of prostate volume. Plan a series of targets to apply the stapler system ahead of time based on, for example, estimated volume, length along the lumen, or other dimensions of the prostate. Build a plan into a surgical robotic control system to provide instructions to autonomously place staples at the target location based on a plan that incorporates real-time 3d feedback from the surgical scene including the target location.

Monitor and provide feedback on tissue deformation during the application of staples by, for example, providing the surgeon with a warning if the staples are being stapled too deeply or too quickly, thereby allowing the surgeon the option to reverse the movement of the surgical robot. 3D feedback can be used to measure one or more of intralumenal diameter, displacement depth, cleared volume of the enlarged prostate, and other variables, to provide feedback as to the effectiveness of the treatment, after the treatment has completed. Real-time 3d visualization can also be provided during the stapling procedure, which is also provided to a 3D display device or wearable headset.

3D information can also be obtained using either our hardware system or RGB-to-3D information tied to the disclosed systems and devices. Measurements can include, for example, straight line distances (e.g. lumen diameter, prostate thickness), surface areas (such as the cross sectional area of the lumen before and after treatment), circumference (also, of the lumen, prostate, or capsule). 3D information can also be combined with fluorescence information, as from ICG, to get quantitative feedback on perfusion (for example, normalize the signal coming from different distances in a given image or across images).

The present teachings may also extend to one or more of the following numbered clauses:

Clause 1. An adapter for use with a lens assembly having a lens assembly proximal end and a lens assembly distal end comprising: a housing mechanically couplable to the lens assembly proximal end wherein the lens assembly contains a continuous illumination path for transmitting light from the lens assembly proximal end to the lens assembly distal end and a continuous optical path relaying a field of view at the lens assembly distal end to an eyepiece at the lens assembly proximal end; and a sensor assembly disposed within the housing of the adapter comprising an optical distance sensor operable to receive a distance-sensing light in a first optical distance wavelength band within a field-of-view via an optical path of the lens assembly.

Clause 2. The adapter of clause 1, wherein the optical distance sensor further comprises a time-of-flight sensor operable to determine a time-delay based distance or a phase shift between an emitted distance-sensing light and a returned distance-sensing light.

Clause 3. The adapter of clause 1, wherein the first optical distance wavelength band is in a visible spectrum.

Clause 4. The adapter of clause 1, wherein the housing further comprises a mechanical interface operable to immobilize the optical distance sensor relative to the lens assembly.

Clause 5. The adapter of clause 1, further comprising an illumination interface, wherein the illumination interface is operable to deliver distance-sensing illumination within the first optical distance wavelength band into the continuous illumination path of the lens assembly, and further wherein the illumination interface is operable to be mechanically couplable in the continuous illumination path of the lens assembly.

Clause 6. The adapter of clause 1, wherein the housing includes a rotational coupler operable to rotate the lens assembly relative to the rotational coupler along a longitudinal axis while allowing measurement of a rotation angle.

Clause 7. The adapter of clause 1, further comprising a six-axis inertial measurement unit, wherein the inertial measurement unit is used to measure the rotation angle of the lens assembly.

Clause 8. The adapter of clause 6, wherein the rotation angle is measured between a stationary handle and the housing during rotation using the rotational coupler positioned between the housing and the handle.

Clause 9. The adapter of clause 1, wherein the optical distance sensor produces an image of a field-of-view for the lens assembly.

Clause 10. The adapter of clause 9, wherein an illumination interface is operable to sequentially transmit a plurality of illumination wavelength bands within a visible spectrum through the illumination path and wherein the optical distance sensor is used to generate a composite color image by imaging sequentially across the plurality of illumination wavelengths bands.

Clause 11. The adapter of clause 1, further comprising a second imaging sensor disposed within the housing, wherein the visible-light imaging sensor is operable to receive visible-wavelength image light in a first visible-light wavelength band from the field of view via the lens assembly optical path and to generate an image data including a distance data corresponding to a second image of the field of view, and the optical distance sensor and the visible-light imaging sensor are arranged so that the distance data and the image data are generated for overlapping portions of the field of view.

Clause 12. The adapter of clause 11, further comprising a beam routing element optically coupled between the optical path of the lens assembly and each of the optical distance sensor and the second imaging sensor, the beam routing element being operable to direct the first optical distance wavelength band to the optical distance sensor and the first visible-light wavelength band to the visible-light imaging sensor.

Clause 13. The adapter of clause 12, wherein the beam routing element comprises a dynamic routing element synchronized with shutter timing of the optical distance sensor and the visible-light imaging sensor.

Clause 14. The adapter of clause 11 wherein the second imaging sensor is a separate and mechanically couplable component to the housing.

Clause 15. The adapter of clause 11, wherein a second camera is rotatable relative to the field of view of the lens assembly.

Clause 16. The adapter of clause 11, wherein the illumination interface is operable to couple distance-sensing illumination at a first optical distance wavelength band and visible-wavelength illumination at a first visible light wavelength band into the illumination path of the lens assembly.

Clause 17. The adapter of clause 16, further comprising a beam combiner, wherein the beam combiner is operable to direct both the first optical distance wavelength band to the first visible-light wavelength band to the illumination path of the lens assembly.

Clause 18. The adapter of clause 16, wherein the illumination interface is operable to couple distance-sensing illumination at a first wavelength band and visible-wavelength illumination obtained from a separate device and transmitted through a separate light guide into a common light path of the lens assembly through a beam combiner.

Clause 19. The adapter of clause 1, further comprising one or more fiducial markers disposed on the housing and operable to be detected by an external tracking system to determine a spatial position of the adapter.

Clause 20. The adapter of clause 1 wherein the lens assembly comprises one of a rigid endoscope and a flexible endoscope that is either reusable or disposable, wherein the endoscope is a laparoscope, an arthroscope, a cystoscope, a ureteroscope, a bronchoscope, a sinuscope, a hysteroscope, a colonoscope, a thoracoscope, or a borescope.

Clause 21. The adapter of clause 1, further comprising a calibration fixture device, the calibration fixture comprising a target portion with a plurality of fiducial markers of known geometry distributed along a surface of known geometry.

Clause 22. The adapter of clause 21, in communication with a calibration fixture wherein the calibration fixture comprises a holder operable to engage the distal end of the lens assembly such that the lens assembly be immobilized relative to the calibration fixture at a known location and such that the target portion is positioned within the field of view of the lens assembly.

Clause 23. The adapter of clause 21, wherein a calibration fixture target portion comprises a plurality of intersecting target planes.

Clause 24. The adapter of clause 21, wherein the fiducial markers are any combination of one or more of optically detectable markers, coded planar markers, chessboard or hybrid calibration patterns, depth-encoded geometric features, reflective markers, and active light-emitting markers.

Clause 25. The adapter of clause 21, wherein the calibration fixture device comprises a fiducial pattern with fiducial pattern geometries that increase in size with increasing distance from a reference position when the fiducial pattern is positioned within the field of view.

Clause 26. The adapter of clause 21 wherein the adapter is further in communication with a fillable vessel with a fluid medium and the calibration fixture encompasses a calibration target surface and a tip of the lens assembly, such that a calibration target is visualized by the lens assembly through the fluid medium.

Clause 27. The adapter of clause 1, wherein the device further includes one or more inertial sensors.

Clause 28. The adapter of clause 27, wherein the inertial sensors include one or more of a plurality of gyroscopes, a plurality of accelerometers, or a plurality of magnetometers.

Clause 29. The adapter of clause 28, wherein the inertial sensors are used to estimate movement information about the device.

Clause 30. The adapter of clause 29, wherein the movement information includes orientation information, wherein the orientation information is used to correct the horizon of the image, such that the orientation of the image on the display corresponds to the orientation of gravity.

Clause 31. The adapter of clause 30, wherein the movement information further includes position information, wherein the position and orientation information is used to digitally combine views from multiple camera angles.

Clause 32. The adapter of clause 31, wherein the combined views are used as a simulated stereo camera to further refine the distance information.

Clause 33. The adapter of clause 32, wherein the combined view are used to stitch the spatial information across the scene together to create a larger spatial representation of the environment than is visible through the field of view of the lens assembly at any given time.

Clause 34. A method for calibrating a system for an optical distance sensor-equipped imaging device, comprising: providing a lens assembly having a lens assembly proximal end and a lens assembly distal end the lens assembly comprises a housing mechanically couplable to the lens assembly proximal end wherein the lens assembly contains a continuous illumination path for transmitting light from the lens assembly proximal end to the lens assembly distal end and a continuous optical path relaying a field of view at the lens assembly distal end to an eyepiece at the lens assembly proximal end, and a sensor assembly disposed within the housing comprising an optical distance sensor operable to receive a distance-sensing light in a first optical distance wavelength band within a field-of-view via an optical path of the lens assembly, wherein the lens assembly disposed comprises a time-of-flight sensor operable to determine a distance based on a time delay or a phase shift between an emitted distance-sensing light and a returned distance-sensing light and is further operable to receive distance-sensing light in a first wavelength band within the field of view via the optical path of the lens assembly, and further wherein an illumination interface is provided that is operable to deliver time-of-flight illumination within the first wavelength band into the illumination path of the lens assembly; providing a calibration fixture device comprising a target portion with a plurality of fiducial markers of known geometry distributed along a surface of known geometry; providing at least one processing circuit operable to receive raw values from a time-of-flight sensor, positioning the lens assembly such that a calibration fixture target portion is positioned within the field of view of the time-of-flight sensor; computing and storing a three-dimensional mapping function for mapping one or more aligned raw values obtained from the time of flight sensor to a corresponding physical three-dimensional coordinate relative to a reference position on the lens assembly; using the three-dimensional mapping function to compute a calibration target surface geometry using the raw values received from the time of flight sensor; computing a plurality of error metrics by spatially correlating a computed calibration target surface geometry with a known calibration target surface geometry and computing their discrepancy; comparing the plurality of error metrics to a corresponding plurality of predetermined acceptance criteria; and conditioning the operability of the optical distance sensor-equipped imaging device for taking geometric measurements of targets disposed within a lens assembly field of view on the acceptability of the error metrics.

Clause 35. The method of clause 34, wherein the raw values obtained from a time-of-flight camera include depth information and intensity information, wherein the depth and intensity information are aligned within the field of view of the lens assembly.

Clause 36. The method of clause 35, wherein the depth information includes a plurality of time delay, phase shift, or uncalibrated physical depth information.

Clause 37. The method of clause 34, wherein the three-dimensional mapping function includes a depth mapping function for mapping one or more of the raw values obtained from the time-of-flight sensor to a physical depth defined along a lens assembly imaging axis relative to a reference point on the lens assembly.

Clause 38. The method of clause 37, wherein the three-dimensional mapping function includes a deprojection function for mapping one or more depth values computed using the depth mapping function to three-dimensional coordinates relative to a reference point on the lens assembly, wherein the deprojection function includes one or more lens parameters including principal point, focal length, skew, and distortion parameters, and wherein the three-dimensional coordinates can be Cartesian, homogeneous, spherical, or cylindrical.

Clause 39. The method of clause 34, wherein the three-dimensional mapping function includes a lens anti-distortion function that corrects optical distortion such that rectilinear targets appear rectilinear in time-of-flight images.

Clause 40. The method of clause 34, wherein the three-dimensional mapping function includes a plurality of corrections for fluid-filled medium, based on a known refractive index for a fluid.

Clause 41. The method of clause 34, wherein the method further computes and indicates a focus of a time-of-flight depth and intensity images.

Clause 42. The method of clause 34, wherein the housing contains an electronic focus mechanism and the processing circuit automatically adjusts the electronic focus mechanism to maximize the focus of a time-of-flight image.

### Clause 43. the Method of Clause 34,

wherein the error metrics includes a surface registration error.

Clause 44. The method of clause 34, wherein the housing further comprises a second camera sensor disposed within the housing, wherein the second camera sensor is operable to receive light in a second wavelength band from the field of view via the lens assembly optical path and to generate image data corresponding to an image of the field of view, and the time-of-flight sensor and the second camera sensor are arranged so that a time-of-flight sensor data and a second sensor data are generated for overlapping portions of the field of view, wherein the method further computes a spatial correlation between the time-of-flight camera sensor and the second camera sensor.

Clause 45. The method of clause 44, wherein the method further computes and indicates a focus of the second camera sensor images.

Clause 46. The method of clause 45, wherein the housing further comprises a second electronic focus mechanism for the second camera and the processing circuit automatically adjusts the electronic focus mechanism to maximize the focus of the second sensor image.

Clause 47. The method of clause 34, wherein the processing circuit computes optimal acquisition parameters of the time-of-flight system.

### Clause 48. the Method of Clause 47,

wherein the acquisition parameters comprise one or more of: integration time, modulation frequency, binning mode, frame rate, pulse timing, pulse duration, peak power, and shutter timing.

Clause 49. The method of clause 34, wherein the calibration fixture further comprises a vessel fillable with a fluid medium, wherein when the vessel is filled with a fluid up to a marked level, the entire optical path between the field of view of the lens assembly and calibration fixture target surface is comprised of the fluid medium.

Clause 50. A guidance system comprising: a housing; a lens assembly disposed within the housing having a lens assembly proximal end and a lens assembly distal end wherein the lens assembly contains a continuous illumination path for transmitting light from the lens assembly proximal end to the lens assembly distal end and a continuous optical path relaying a field of view at the lens assembly distal end to an eyepiece at the lens assembly proximal end; and an assembly disposed within the housing comprising a time-of-flight sensor operable to determine a distance based on a time delay or a phase shift between an emitted distance-sensing light and a returned distance-sensing light, wherein the time-of-flight sensor is operable to receive distance-sensing light in a first wavelength band within the field of view via the optical path of the lens assembly; an illumination interface disposed within the housing, wherein the illumination interface is operable to deliver time-of-flight illumination within the first wavelength band into the illumination path of the lens assembly; a calibration fixture device, the calibration fixture comprising a target portion with a plurality of fiducial markers of known geometry distributed along a surface of known geometry; at least one processing circuit operable to receive raw values from the time-of-flight sensor; at least one display operable to receive and render data from the processing unit; a processor operable to compute and store a three-dimensional mapping function for mapping one or more aligned raw values obtained from the time of flight sensor to a corresponding physical three-dimensional coordinate relative to a reference position on the lens assembly, wherein the guidance system is used to measure three-dimensional locations of a plurality of target points visible within the field of view of the lens assembly, wherein the processing circuit and the processor are the same.

Clause 51. The guidance system of clause 50 wherein the guidance system is a surgical guidance system.

Clause 52. The guidance system of clause 50 wherein the housing further comprises a second camera sensor disposed within the housing, wherein the second camera sensor is operable to receive light in a second wavelength band from the field of view via the lens assembly optical path and to generate image data corresponding to an image of the field of view, and the time-of-flight sensor and the second camera sensor are arranged so that the time-of-flight sensor data and the second sensor data are generated for overlapping portions of the field of view, wherein the calibration process is used to establish a spatial correlation of the time of flight sensor field of view to the second camera field of view, wherein the three-dimensional location information from the time-of-flight sensor is thereby spatially correlated to information from the second camera sensor.

Clause 53. The guidance system of clause 52 wherein the three-dimensional location information is used to normalize a plurality of intensity values in the image obtained from the second camera sensor.

Clause 54. The guidance system of clause 53, wherein the normalization compensates for distance-dependent intensity attenuation proportional to an inverse square of a distance between the system and objects in the field of view.

Clause 55. The guidance system of clause 53 wherein the three dimensional location information is displayed as an overlay on the time-of-flight sensor image, wherein the overlay comprises a plurality of sets of points, each of the plurality of sets of points comprising points that are equidistant from a reference point associated with a tip of the endoscope along at least one spatial or angular component.

Clause 56. The guidance system of clause 53 wherein the processor is operable to allow the user to measure a plurality of geometric parameters, including one or more of: the location of a single selected point; the length or angle of a line segment defined by two selected points; the angle between two line segments; the length of a curved path; the circumference, surface area, projected area, or enclosed volume of a closed contour; and an orientation, pose, deviation, or offset relative to a reference geometry.

Clause 57. The guidance system of clause 53 wherein the system further comprises an externally obtained three-dimensional scan of targets visible within the field of view of the lens assembly and wherein the system is operable to perform markerless three-dimensional spatial correlation between the intra-procedure three-dimensional location information and a corresponding surface on the externally obtained three-dimensional scan.

Clause 58. The guidance system of clause 57 wherein the externally obtained three-dimensional scan is a computed tomography, positron emission tomography, ultrasonic, optical coherence tomography, or magnetic resonance imaging scan.

Clause 59. The guidance system of clause 57, wherein the three-dimensional spatial correlation is used to infer the locations of structures present in the externally obtained three-dimensional scan but not visible in the field of view of the lens assembly, and the inferred locations of the structures not visible in the field of view are displayed as overlays.

Clause 60. The guidance system of clause 57, wherein the computation of three-dimensional spatial correlation automatically excludes portions of the field of view in which there is minor surface modification or occlusion, wherein the three-dimensional spatial correlation is made to be robust in the presence of minor surface modification or occlusion.

Clause 61. The guidance system of clause 57, wherein the computation of the three-dimensional spatial correlation accounts for changes to one or more structures represented in the externally obtained three-dimensional scan during a procedure, including one or more of deformation, displacement, separation, removal, or modification of the anatomical structures, such that the three-dimensional spatial correlation remains robust in the presence of procedural alteration of the structures.

Clause 62. The guidance system of clause 57, wherein the display comprises a two-dimensional monitor, stereoscopic display, or wearable head-mounted display.

Clause 63. The guidance system of clause 62, wherein the housing further contains a plurality of fiducial markers rigidly disposed on the housing; wherein the plurality of fiducial markers are arranged in known spatial relationships relative to the lens assembly, and further wherein an external tracking system is used to track the plurality of fiducial markers and thereby localize the three-dimensional spatial information obtained through the field of view of the lens assembly within a coordinate system referenced to the external tracking system.

Clause 64. The guidance system of clause 63, wherein the external tracking system is rigidly mounted on a wearable head-mounted display, wherein the navigation system generates an overlay showing the target location within the reference system of the wearable display, thus enabling the sensation of subsurface visualization.

Clause 65. The guidance system of clause 50, wherein the three-dimensional spatial correlation is used to localize the externally obtained three-dimensional scan within a common coordinate system referenced to the lens assembly.

Clause 66. The guidance system of clause 65, further comprising one or more instruments, each instrument including a set of fiducial markers arranged in known spatial relationships relative to one or more instrument features of interest, wherein the guidance system is configured to detect the fiducial markers in image data captured by the lens assembly and determine a pose of each instrument within the common coordinate system, and wherein the guidance system generates real-time guidance information indicating three-dimensional spatial relationships between the instrument features of interest and the registered externally obtained three-dimensional scan.

Clause 67. The guidance system of clause 66, wherein the instrument comprises one or more of a drill, stapler, scalpel, cutting instrument, probe, needle, catheter, or implant delivery instrument.

Clause 68. The guidance system of clause 67, wherein the instrument is controlled by a robotic manipulator using movement cues derived from geometric data.

Clause 69. The guidance system of clause 63, further comprising one or more instruments, wherein the instrument position and orientation are tracked using machine vision techniques and a-priori knowledge of the instrument geometry, or with the spatial information provided by the optical distance sensor.

Clause 70. The guidance system of clause 57, wherein the device further contains a robotic manipulator operable to hold and move at least one of the optical imaging device and a surgical instrument; and a robotic controller operable to receive the three-dimensional location information obtained through the field of view of the lens assembly spatially correlate this information with the surgical instrument with a common three-dimensional coordinate system; and further wherein the robotic controller is operable to receive one or more navigation cues and to apply at least one motion constraint or motion command to the robotic manipulator based on the common three-dimensional coordinate system.

Clause 71. The guidance system of clause 70, wherein the robotic controller enforces one or more virtual fixtures, safe corridors, or exclusion volumes defined relative to an anatomical structure in the pre-procedure scan, in which the pre-procedure scan was localized to the common coordinate system using three-dimensional spatial correlation obtained using three-dimensional location information obtained from the field of view of the lens assembly.

Clause 72. The guidance system of clause 71, wherein the robotic controller is operable to halt, limit, or redirect motion of the robotic manipulator when a distance between a tool and an anti-target region falls below a threshold determined from three dimensional location information.

Clause 73. The guidance system of clause 72, wherein the robotic controller is operable to autonomously move the optical imaging device or instrument along a pre-planned trajectory defined in the common three-dimensional coordinate system, wherein the instrument includes a one or more of a drill, stapler, scalpel, cutting instrument, probe, needle, catheter, or implant delivery instrument.

Clause 74. A computer-implemented method of training a model for pre-procedure preparation, comprising: a. obtaining a training dataset comprising i. pre-procedure scans, ii. intra-procedure depth maps and visible-light images acquired through an optical distance sensor and visible light sensor-equipped imaging device, and iii. procedure plans and associated outcomes; b. training a machine learning model to learn a mapping from the pre-procedure scans and the intra-procedure depth maps to one or more planning outputs selected from: recommended target locations, recommended trajectories, safety margins, and risk scores; and c. storing parameters of the trained machine learning model operable to infer the planning outputs from a new pre-procedure scan.

Clause 75. The method of clause 74, further comprising normalizing intensity or fluorescence signals in the visible-light images using distance from the depth maps and using the normalized signals as input features to the machine learning model.

Clause 76. The method of clause 74, wherein the machine learning model comprises at least one of a convolutional neural network, a three-dimensional convolutional neural network, a transformer-based architecture, a recurrent neural network, neural radiance fields, or Gaussian splats.

Clause 77. The method of clause 74, further comprising disabling or limiting at least one navigation function when a deviation between the newly computed calibration data and the previously stored calibration data exceeds a threshold.

Clause 78. A non-transitory computer-readable medium storing parameters of the model trained according to the method of clause 74 and instructions to apply the model to automatically propose, for a new case, one or more target locations and trajectories displayable within a navigation user interface for user acceptance or modification.

Clause 79. An arthroscopic guidance system, comprising: a. an arthroscope having a rod-lens optical path, an illumination channel, and an instrument channel; b. an optical distance sensor optically coupled to the rod-lens optical path and operable to generate a depth map of an intra-articular field of view; c. a visible-light imaging sensor optically coupled to the rod-lens optical path; and d. a processing circuit operable to register the depth map to a pre-procedure scan of a joint and to display a navigation view including at least one planned tunnel trajectory overlaid relative to articular surfaces.

Clause 80. The system of clause 79, wherein the planned tunnel trajectory corresponds to an anterior cruciate ligament graft tunnel.

Clause 81. A laparoscopic guidance system, comprising: a. a laparoscope having a tubular member, a distal tip with digital optics, and a working channel; b. an optical distance sensor operable to generate a depth map of an intra-abdominal field of view; c. a processing circuit operable to register the depth map to a pre-procedure abdominal scan, of a target anatomical surface during a partial nephrectomy procedure, and to compute distances to critical structures along a planned stapling or cutting line; and d. a display operable to overlay the planned stapling or cutting line and safety margins relative to the critical structures in the field of view.

Clause 82. An endoscopic guidance system, comprising: a. a flexible endoscope having a distal tip, an insertion tube, an illumination channel, and an instrument channel; b. an optical distance sensor operable to generate depth maps along a lumen; c. a processing circuit operable to compute at least one of: intraluminal diameter, surface area of mucosa, or lesion size from the depth maps; and d. a display operable to present a luminal navigation view with quantitative measurements.

Clause 83. A urologic guidance system, comprising: a. a cystoscope or ureteroscope having a distal tip configured for intraluminal placement in a urinary tract; b. an optical distance sensor operable to generate a depth map of a prostatic urethra or ureter; c. a processing circuit operable to compute at least one of: urethral length, prostate thickness, capsule distance, intraluminal diameter, or cleared volume, and to define target locations for placement of one or more of a staple, implant, and stent; and d. a display operable to overlay the target locations on a depth-annotated endoscopic image.

Clause 84. A bronchoscopic guidance system, comprising: a. a bronchoscope; b. an optical distance sensor operable to generate point clouds or 3D meshes of airway lumens and branch points; and c. a processing circuit operable to register the point clouds or 3D meshes to a bronchial tree model and to guide navigation to a target lesion with branch-level waypoints.

Clause 85. A sinus guidance system, comprising: a. a sinuscope; b. an optical distance sensor operable to generate point clouds or 3D meshes of sinus cavities and adjacent bony structures; and c. a processing circuit operable to display an overlay indicating the position of the skull base, orbit, nerves, or other critical structures not visible in the native endoscope field of view and to enforce safety margins during sinus surgery.

Clause 86. An industrial borescope guidance system, comprising: a. a borescope configured for insertion into an industrial asset; b. an optical distance sensor operable to generate depth maps of internal surfaces while the borescope is within a coolant, process liquid, or gas; and c. a processing circuit operable to register the depth maps to a three-dimensional model of the asset and to compute dimensions of defects, deposits, or wear features.

Clause 87. The system of clause 72, further comprising a robotic manipulator operable to move the borescope along a planned inspection path while respecting motion constraints derived from the depth maps.

As will be appreciated by those skilled in the art, throughout this specification, unless the context requires otherwise, the word “comprise” or variations such as “comprises” or “comprising”, will be understood to imply the inclusion of a stated integer or group of integers but not the exclusion of any other integer or group of integers.

Although these teachings have been described with respect to various examples, it should be appreciated that these teachings are also capable of a wide variety of further and other examples within the spirit and scope of these teachings. Thus, although an overview of the subject matter has been described with reference to specific examples, various modifications and changes may be made to these examples without departing from the broader scope of the present disclosure. It will also be obvious to those skilled in the art that the examples are provided by way of example only. Numerous variations, changes, and substitutions will occur to those skilled in the art without departing from the scope of the disclosure. It should also be understood that various alternatives may be employed in practicing the disclosed innovation. It is intended that any claims presented at any time in this application, or a subsequent application, define the scope of the invention and that methods and structures within the scope of any claims and their equivalents are covered thereby.

What is claimed:

1. An adapter for use with a lens assembly having a lens assembly proximal end and a lens assembly distal end comprising:
a housing mechanically couplable to the lens assembly proximal end wherein the lens assembly contains a continuous illumination path for transmitting light from the lens assembly proximal end to the lens assembly distal end and a continuous optical path relaying a field of view at the lens assembly distal end to an eyepiece at the lens assembly proximal end; and
a sensor assembly disposed within the housing of the adapter comprising an optical distance sensor operable to receive a distance-sensing light in a first optical distance wavelength band within a field-of-view via an optical path of the lens assembly.

2. The adapter of claim 1, wherein the optical distance sensor further comprises a time-of-flight sensor operable to determine a time-delay based distance or a phase shift between an emitted distance-sensing light and a returned distance-sensing light.

3. The adapter of claim 1, wherein the first optical distance wavelength band is in a visible spectrum.

4. The adapter of claim 1, wherein the housing further comprises a mechanical interface operable to immobilize the optical distance sensor relative to the lens assembly.

5. The adapter of claim 1, further comprising an illumination interface, wherein the illumination interface is operable to deliver distance-sensing illumination within the first optical distance wavelength band into the continuous illumination path of the lens assembly, and further wherein the illumination interface is operable to be mechanically couplable in the continuous illumination path of the lens assembly.

6. The adapter of claim 1, wherein the housing includes a rotational coupler operable to rotate the lens assembly relative to the rotational coupler along a longitudinal axis while allowing measurement of a rotation angle.

7. The adapter of claim 1, further comprising a six-axis inertial measurement unit, wherein the inertial measurement unit is used to measure a rotation angle of the lens assembly.

8. The adapter of claim 1, wherein the optical distance sensor produces an image of a field-of-view for the lens assembly.

9. The adapter of claim 1, further comprising a second imaging sensor disposed within the housing, wherein the second imaging sensor is operable to receive visible-wavelength image light in a first visible-light wavelength band from the field of view via the lens assembly optical path and to generate an image data including a distance data corresponding to a second imaging sensor image field of view, and the optical distance sensor and the second imaging sensor are arranged so that the distance data and the image data are generated for overlapping portions of the field of view.

10. The adapter of claim 1, further comprising one or more fiducial markers disposed on the housing and operable to be detected by an external tracking system to determine a spatial position of the adapter.

11. The adapter of claim 1 wherein the lens assembly comprises one of a rigid endoscope and a flexible endoscope that is either reusable or disposable, wherein the endoscope is a laparoscope, an arthroscope, a cystoscope, a ureteroscope, a bronchoscope, a sinuscope, a hysteroscope, a colonoscope, a thoracoscope, or a borescope.

12. The adapter of claim 1, further comprising a calibration fixture device, the calibration fixture comprising a target portion with a plurality of fiducial markers of known geometry distributed along a surface of known geometry.

13. The adapter of claim 1, wherein the adapter further includes one or more inertial sensors.

14. A method for calibrating a system for an optical distance sensor-equipped imaging device, comprising:
providing a lens assembly having a lens assembly proximal end and a lens assembly distal end the lens assembly comprises a housing mechanically couplable to the lens assembly proximal end wherein the lens assembly contains a continuous illumination path for transmitting light from the lens assembly proximal end to the lens assembly distal end and a continuous optical path relaying a field of view at the lens assembly distal end to an eyepiece at the lens assembly proximal end, and a sensor assembly disposed within the housing comprising an optical distance sensor operable to receive a distance-sensing light in a first optical distance wavelength band within a field-of-view via an optical path of the lens assembly, wherein the lens assembly disposed comprises a time-of-flight sensor operable to determine a distance based on a time delay or a phase shift between an emitted distance-sensing light and a returned distance-sensing light and is further operable to receive distance-sensing light in a first wavelength band within the field of view via the optical path of the lens assembly, and further wherein an illumination interface is provided that is operable to deliver time-of-flight illumination within the first wavelength band into the illumination path of the lens assembly;
providing a calibration fixture device comprising a target portion with a plurality of fiducial markers of known geometry distributed along a surface of known geometry;
providing at least one processing circuit operable to receive raw values from a time-of-flight sensor, positioning the lens assembly such that a calibration fixture target portion is positioned within the field of view of the time-of-flight sensor;
computing and storing a three-dimensional mapping function for mapping one or more aligned raw values obtained from the time of flight sensor to a corresponding physical three-dimensional coordinate relative to a reference position on the lens assembly;
using the three-dimensional mapping function to compute a calibration target surface geometry using the raw values received from the time of flight sensor;
computing a plurality of error metrics by spatially correlating a computed calibration target surface geometry with a known calibration target surface geometry and computing their discrepancy;
comparing the plurality of error metrics to a corresponding plurality of predetermined acceptance criteria; and
conditioning the operability of the optical distance sensor-equipped imaging device for taking geometric measurements of targets disposed within a lens assembly field of view on the acceptability of the error metrics.

15. The method of claim 14, wherein the raw values obtained from a time-of-flight camera include depth information and intensity information, wherein the depth and intensity information are aligned within the field of view of the lens assembly.

16. The method of claim 14, wherein the three-dimensional mapping function includes a depth mapping function for mapping one or more of the raw values obtained from the time-of-flight sensor to a physical depth defined along a lens assembly imaging axis relative to a reference point on the lens assembly.

17. The method of claim 14, wherein the three-dimensional mapping function includes a lens anti-distortion function that corrects optical distortion such that rectilinear targets appear rectilinear in time-of-flight images.

18. The method of claim 14, wherein the three-dimensional mapping function includes a plurality of corrections for fluid-filled medium, based on a known refractive index for a fluid.

19. The method of claim 14, wherein the method further computes and indicates a focus of a time-of-flight depth and intensity images.

20. The method of claim 14, wherein the housing contains an electronic focus mechanism and the processing circuit automatically adjusts the electronic focus mechanism to maximize the focus of a time-of-flight image.

21. The method of claim 14, wherein the error metrics includes a surface registration error.

22. The method of claim 14, wherein the housing further comprises a second camera sensor disposed within the housing, wherein the second camera sensor is operable to receive light in a second wavelength band from the field of view via the lens assembly optical path and to generate image data corresponding to an image of the field of view, and the time-of-flight sensor and the second camera sensor are arranged so that a time-of-flight sensor data and a second sensor data are generated for overlapping portions of the field of view, wherein the method further computes a spatial correlation between the time-of-flight sensor and the second camera sensor.

23. The method of claim 14, wherein a processing circuit computes optimal acquisition parameters of a time-of-flight system.

24. The method of claim 14, wherein the calibration fixture further comprises a vessel fillable with a fluid medium, wherein when the vessel is filled with a fluid up to a marked level, an entire optical path between the field of view of the lens assembly and calibration fixture target surface is comprised of the fluid medium.

25. A guidance system comprising:
a housing;
a lens assembly disposed within the housing having a lens assembly proximal end and a lens assembly distal end wherein the lens assembly contains a continuous illumination path for transmitting light from the lens assembly proximal end to the lens assembly distal end and a continuous optical path relaying a field of view at the lens assembly distal end to an eyepiece at the lens assembly proximal end;
an assembly disposed within the housing comprising a time-of-flight sensor operable to determine a distance based on a time delay or a phase shift between an emitted distance-sensing light and a returned distance-sensing light, wherein a time-of-flight sensor is operable to receive distance-sensing light in a first wavelength band within the field of view via the optical path of the lens assembly;
an illumination interface disposed within the housing, wherein the illumination interface is operable to deliver time-of-flight illumination within the first wavelength band into the illumination path of the lens assembly;
a calibration fixture device, the calibration fixture comprising a target portion with a plurality of fiducial markers of known geometry distributed along a surface of known geometry;
at least one processing circuit operable to receive raw values from the time-of-flight sensor; at least one display operable to receive and render data from the processing circuit; and
a processor operable to compute and store a three-dimensional mapping function for mapping one or more aligned raw values obtained from the time of flight sensor to a corresponding physical three-dimensional coordinate relative to a reference position on the lens assembly, wherein the guidance system is used to measure three-dimensional locations of a plurality of target points visible within the field of view of the lens assembly.

26. The guidance system of claim 25 wherein the guidance system is a surgical guidance system.

27. The guidance system of claim 25 wherein the housing further comprises
a second camera sensor disposed within the housing, wherein the second camera sensor is operable to receive light in a second wavelength band from the field of view via a lens assembly optical path and to generate image data corresponding to an image of the field of view, and
a time-of-flight sensor and the second camera sensor are arranged so that a time-of-flight sensor data and a second sensor data are generated for overlapping portions of the field of view, wherein a calibration process is used to establish a spatial correlation of the time of flight sensor field of view to a second camera field of view, wherein a three-dimensional location information from the time-of-flight sensor is thereby spatially correlated to information from the second camera sensor.

28. A computer-implemented method of training a model for pre-procedure preparation, comprising:
a. obtaining a training dataset comprising
i. pre-procedure scans,
ii. intra-procedure depth maps and visible-light images acquired through an optical distance sensor-equipped imaging device, and
iii. procedure plans and associated outcomes;
b. training a machine learning model to learn a mapping from the pre-procedure scans and the intra-procedure depth maps to one or more planning outputs selected from: recommended target locations, recommended trajectories, safety margins, and risk scores; and
c. storing parameters of trained machine learning model operable to infer the planning outputs from a new pre-procedure scan.

29. An arthroscopic guidance system, comprising:
a. an arthroscope having a rod-lens optical path, an illumination channel, and an instrument channel;
b. an optical distance sensor optically coupled to the rod-lens optical path and operable to generate a depth map of an intra-articular field of view;
c. a visible-light imaging sensor optically coupled to the rod-lens optical path; and
d. a processing circuit operable to register the depth map to a pre-procedure scan of a joint and to display a navigation view including at least one planned tunnel trajectory overlaid relative to articular surfaces.
